SEQUENCE LISTING (5 sequences)

SEQ ID NO: 1 - DNA, 22 bp, Artificial Sequence
  HMGB1 Forward primer
tgtcatgccaccctgagcagtt

SEQ ID NO: 2 - DNA, 20 bp, Artificial Sequence
  HMGB1 Reverse primer
tgtgctcctcccggcaagtt

SEQ ID NO: 3 - DNA, 20 bp, Artificial Sequence
  Common Forward primer
aagggagctgcagtggagta

SEQ ID NO: 4 - DNA, 20 bp, Artificial Sequence
  Wild Type Reverse primer
ccgaaaatctgtgggaagtc

SEQ ID NO: 5 - DNA, 20 bp, Artificial Sequence
  Mutant Reverse primer
cggttattcaacttgcacca

(19) United States
(12) Patent Application Publication
(10) Pub. No.: US 2022/0023387 A1
(43) Pub. Date: Jan. 27, 2022

(54) PROPHYLACTIC AND THERAPEUTIC USES OF FULLY REDUCED FORMS OF HMGB1 IN CONDITIONS INVOLVING ORGANS

(71) Applicant: Oxford University Innovation Limited, Oxford (GB)

(72) Inventors: Jagdeep Nanchahal, Oxford (GB); Ana Isabel Espirito Santo, Oxford (GB); Geoffrey Lee, Darlinghurst (AU); James Chan, Oxford (GB); Nicole Horwood, Oxford (GB); Marc Feldmann, London (GB)

(73) Assignee: Oxford University Innovation Limited, Oxford (GB)

(21) Appl. No.: 17/046,711

(22) PCT Filed: Apr. 9, 2019
(86) PCT No.: PCT/IB2019/000385

§ 371 Date: Oct. 9, 2020

Related U.S. Application Data
(60) Provisional application No. 62/655,748, filed on Apr. 10, 2018.

(51) Int. Cl. A61K 38/19

(52) U.S. Cl. CPC A61K 38/19

(57) ABSTRACT

The subject invention provides a method of preventing or treating a condition associated with a defect in, or damage to, an organ in a subject with, or at risk for, such defect or damage to such organ which comprises administering to the subject a therapeutically effective amount of the fully reduced (all thiol) form of HMGB1 or a biologically active truncated form of HMGB1, so as to prevent or treat such condition.

The subject invention also provides a method of improving regeneration of blood in a subject comprising administering a therapeutically effective amount of the fully reduced (all thiol) form of HMGB1 or a biologically active truncated form of HMGB1, effective to improve regeneration of blood.

### CROSS-REFERENCE TO RELATED APPLICATIONS

This application is a § 371 national stage of PCT International Application No. PCT/IB2019/000385, filed Apr. 9, 2019, claiming the benefit of U.S. Provisional Application No. 62/655,748, filed Apr. 10, 2018, the contents of each of which are hereby incorporated by reference.

Throughout this application various publications are referenced by the last name of the first author and the year of publication. Full citations for these publications are set forth in a section entitled References immediately preceding the claims. The disclosures of all referenced publications in their entireties are hereby incorporated by reference into this application in order to more fully describe the state of the art to which the invention relates.

### REFERENCE TO SEQUENCE LISTING

This application incorporates-by-reference nucleotide and/or amino acid sequences which are present in the file named “210506_90170-PCT-US_Sequence_Listing_AWG.txt,” which is 1.34 kilobytes in size, and which was created Apr. 5, 2021 in the IBM-PC machine format, having an operating system compatibility with MS-Windows, which is contained in the text file filed May 6, 2021 as part of this application.

### BACKGROUND OF INVENTION

Adult stem cells are an essential component of tissue homeostasis with indispensable roles in both physiological tissue renewal and tissue repair following injury (Weissman 2000). The regenerative potential of stem cells has been very successful for haematological disorders (Gratwohl 2015). In contrast, there has been comparatively little clinical impact on enhancing the regeneration of solid organs despite the continuing major scientific and public interest (Brooks 2017). Strategies that rely on ex vivo expansion of autologous stem cells on an individual patient basis are prohibitively expensive (Trainor 2014) and success in animal models has often failed to translate in late phase clinical trials. The use of allogeneic cells would overcome the problems of limited supply but commonly entails risky lifelong immunosuppressive therapy. Some safety concerns remain about induced pluripotent stem cells (Dimmeler 2014). Furthermore, successful engraftment of exogenous stem cells to sites of tissue injury requires a supportive inductive niche and the typical proinflammatory scarred bed in damaged recipient tissues is sub-optimal (Forbes 2014) and cells that do engraft appear to largely act by release of paracrine factors rather than functional replacement of damaged cells (Ilic 2012).

An attractive alternative strategy, which overcomes many of the limitations described above, is to promote repair by directly harnessing the regenerative potential of endogenous stem cells (Dimmeler 2014, Lane 2014). This requires identification of key soluble mediators that enhance the activity of stem cells and can be administered systemically (Zhang 2015, Smith 2017). An interesting observation was made in 1970 that a priming injury at a distant site at the time or before the second trauma resulted in accelerated healing (Joseph 1970, Davis 2005). This phenomenon was only explained recently, when it was shown that a soluble mediator is released following the priming tissue injury which transitions stem cells in the contralateral limb to a state the authors termed G_{Alert} (Rodgers 2014), which is intermediate between G₀ and G₁. In the presence of activating factors the primed G_{Alert} cells enter the cell cycle more rapidly than quiescent stem cells, leading to accelerated tissue repair (Rodgers 2014). However, the identity of the soluble mediator(s) that transition stem cell to G_{Alert} remain to be clarified.

### SUMMARY OF THE INVENTION

The subject invention provides a method of preventing or treating a condition associated with a defect in, or damage to, an organ in a subject with, or at risk for, such defect or damage to such organ which comprises administering to the subject an amount of either (a) the fully reduced (all thiol) form of HMGB1, or (b) a truncated form of HMGB1 having the biological activity of the fully reduced form of HMGB1, effective to prevent or treat such condition.

The subject invention also provides a method of improving regeneration of blood in a subject in need thereof which comprises administering to the subject an amount of either (a) the fully reduced (all thiol) form of HMGB1, or (b) a truncated form of HMGB1 having the biological activity of the fully reduced form of HMGB1, effective to improve regeneration of blood in the subject.

### BRIEF DESCRIPTION OF THE FIGURES

FIG. 1: Alarmins are elevated post-injury in humans and mice, and HMGB1 primes human MSCs forosteogenic differentiation. (A, B) Elevated plasma levels of S100A8/A9 and HMGB1 post-femoral fracture in patients (A) and mice (B), collected within 4 hours, and at 3 hours post-fracture respectively (n=15 fractured and 15 unfractured human patients, and 6 unfractured and 4 fractured mice) (C) Results of in vitro osteogenesis screen of alarmins using human MSC and monocytes represented as a heat map. Green=elevated, red=reduced, black=unchanged; colour brightness indicates dose trend. G indicates that the box is colored Green, LG means light green and DG means dark green. R indicates that the box is colored Red, LR means light red and DR means dark red. All data shown and quantified in FIG. 6. (D-F), Osteogenic differentiation is unchanged in hMSCs primed with S100A8 (D), but increased when primed with FR (E), or 3S-HMGB1 (F), as measured by ALP activity (n=3 hMSC and 3 monocyte donors for each condition, with similar results in 3 independent experiments; significance is versus OM control). DM=maintenance media, OM=osteogenic media.

FIG. 2: HMGB1 accelerates fracture healing via CXCL12-CXCR4. (A, B) Local addition of FR or 3S-HMGB1 accelerates fracture healing, compared to CXCL12 or vehicle controls, as shown by in vivo microCT radiographs (A), and analysis of callus volume, callus bone mineral density (BMD) and day 28 mechanical strength (B) (n=10 mice for each condition). (C) Hmgb1^{-/-} mice have markedly delayed fracture healing compared to Hmgb1^{F1/F1} control mice as shown by reduced callus volume, callus BMD and day 28 mechanical strength (n=7 Hmgb1^{-/-} mice, 8 Hmgb1^{f1/f1} mice). (D, E) Elevated plasma levels of HMGB1-CXCL12 heterocomplex post-fracture in patients (D) and mice (E), collected within 4 hours, and at 3 hours post-fracture respectively, and inhibition of HMGB1-CXCL12 heterocomplex formation with glycyrrhizin treatment (n=15 fractured and 15 unfractured human patients, and 6 unfractured and 4 fractured mice). (F, G) Glycyrrhizin delays fracture healing compared to vehicle controls (F), and AMD3100 abrogates the effects of exogenous FR or 3S-HMGB1 (G) as shown by callus volume, callus BMD and day 28 mechanical strength (n=10 mice for each condition). (H, I) mSSCs express functional surface CXCR4 as shown by FACS histogram plot (H) (n=4 mice for each condition), and time-lapse microscopy trajectory plots of mSSCs migrating to CXCL12, or 0% or 20% FBS control (I) (n=50 cells for each condition, with similar results observed in 3 independent experiments; DMEM=Dulbecco's Modified Eagle Medium).

FIG. 3: HMGB1 transitions stem cells to G_{Alert}. (A) mSSCs from animals treated locally with exogenous FR or 3S-HMGB1 dynamically adapted to the known physiologically rising levels of activating factors with a sustained higher propensity to cycle (n=4 mice for each condition and time point). (B) Effects of exogenous FR or 3S-HMGB1 are mTORC1 dependent in vivo because they are abrogated with rapamycin treatment, as shown by callus volume, callus BMD and day 28 mechanical strength (n=10 mice for each condition). (C) mSSCs, mHSCs, and mMuSCs from the limb contralateral to fracture (fracture (#) alerted) of Hmgb1^{-/-} mice display equivalent ATP levels as quiescent cells from uninjured Hmgb1^{-/-} and Hmgb1^{f1/f1} mice (n=4 mice for each condition). (D-F) mSSCs, mHSCs, and mMuSCs from mice treated systemically with FR or 3S-HMGB1 display increased cellular ATP levels (D) mitochondrial DNA (E) (n=4 mice for each condition, separate experiments for each parameter), and cell size (F) (n=100 cells for each condition, with similar results observed in 3 independent experiments from n=4 mice per condition) compared to vehicle controls, and equivalent to fracture #alerted cells. (G) mSSCs, mHSCs, and mMuSCs from mice treated systemically with FR or 3S-HMGB1 display faster entry to cell cycle, but slower than cells from the ipsilateral limb to the fracture (fracture (#) activated) cells (n=4 mice for each condition and time point).

FIG. 4: HMGB1 accelerates healing of multiple tissues, even if administered 2 weeks before injury. (A-C) Systemic administration of FR or 3S-HMGB1 accelerates haematopoietic recovery following 5-FU myeloablation (A), as shown by peripheral leucocyte (B) and neutrophil counts (C) (n=8 mice each for FR and 3S-HMGB1 conditions, 9 mice for vehicle controls). (D-F) Local administration of FR or 3S-HMGB1 accelerates muscle regeneration following BaCl₂ injury (D), as shown by increased muscle fibre cross sectional area (CSA) (E and F) (n=4 for each condition and time point). (G-M) Systemic administration of FR or 3S-HMGB1 2 weeks prior to injury accelerates: fracture healing (G) as shown by in vivo microCT radiographs (H), callus volume, callus BMD and mechanical strength (I) (n=10 mice for each condition); haematopoietic recovery (J) as shown by peripheral leucocyte (K) and neutrophil counts (L) (n=8 mice each for FR and 3S-HMGB1 conditions, 9 mice for vehicle controls); and muscle regeneration (J) as shown by increased muscle fibre cross sectional area (CSA) (M) (n=5 mice for each condition and time point). (N) Schematic of dynamic and adaptive HMGB1-CXCL12-CXCR4-G_{Alert} accelerated tissue regeneration pathway.

FIG. 5: Time course of alarmins post-fracture and schematic of redox states and functions of HMGB1. (A, B) Circulating levels of S100A8/A9 (A) and HMGB1 (B) in plasma after femoral fracture over 28 days. Plasma samples were collected at 1 h, 3 h, 6 h, 10 h, 5 d, 7 d and 28 d post-fracture and from unfractured mice (n=4 mice for each condition and time point). (C) HMGB1 function is dependent on the redox status. Nuclear HMGB1 is fully reduced and in this state extracellular HMGB1 enhances the chemotactic activity of CXCL12 by forming a heterocomplex with this chemokine and binding to the receptor CXCR4. Fully reduced HMGB1 can be oxidized to the disulfide form, which is proinflammatory but has no chemotactic activity. Fully oxidized HMGB1 is inert. Substitution of cysteines at C23, C45 and C106 by serines prevents oxidation and the molecule behaves as in the all thiol fully-reduced form.

FIG. 6: Full human MSC and monocyte osteogenesis screen. (A) Only human monocytes treated with LPS, S100A8, S100A9, or DS-HMGB1 show elevated levels of TNF, significance is versus RPMI control. (B) No alarmin affects osteogenic differentiation when added directly to hMSCs in OM, significance is versus OM control. (C, D) Monocytes co-cultured with hMSCS (C) or supernatant from human monocytes (D), treated with LPS, S100A8, S100A9, or DS-EMGB1 inhibit osteogenic differentiation of hMSCs in a dose-dependent manner, significance is versus OM control. (E) Only hMSCs primed with FR or 3S-HMGB1 show increased osteogenic differentiation, significance is versus OM control. n=3 hMSC and 3 monocyte donors for each condition for all experiments (A-E), with similar results in 3 independent experiments. (F) Heat map representation of complete in vitro osteogenesis screen of alarmins, compared to a PAMP, with circulating alarmins post-injury, using human plasma, hMSCs and monocytes. Green=elevated, red (R)=reduced, black=unchanged, grey=not applicable or not done; colour brightness indicates dose trend. G indicates that the box is colored Green, LG means light green and DG means dark green. R indicates that the box is colored Red, LR means light red and DR means dark red. DM=maintenance media, OM=osteogenic media, OSM=oncostatin M.

FIG. 7: Fracture healing model, analysis and HMGB1 dose response. FR and 3S-HMGB1 do not induce proinflammatory cytokine production in vivo, and local exogenous addition of CXCL12 increases cell migration to the fracture site. (A-C) Murine femur fracture model shown with illustrations and 3D microCT reconstruction (A), external fixator in situ (B), and schematic of region of interest (C). (D, E) Best curve fitting of callus volume data (D) with mathematical modelling and F-test (E). (F, G) Mechanical strength testing apparatus setup (F) and assessment (G). (H) Mice treated locally with 3S-HMGB1 show improved fracture healing by mechanical strength testing in a dose-dependent manner, with a plateau in efficacy at 0.75 mg/kg (n=10 mice for each condition). (I-K) TNF, IL-6 and IL-10 levels are equivalent to vehicle controls after i.v. administration of FR or 3S-HMGB1. DS-HMGB1 and LPS used as positive controls and resulted in elevated levels of all three cytokines as expected. Plasma samples were collected at 0.5 h, 1 h, 3 h, 18 h, 48 h and 2 weeks (n=4 mice for each condition and time point). (L) Local administration of CXCL12 resulted in increased migration of cells to the fracture site 12 h post-injury as shown by more non-cycling (BrdU⁻) cells per fractured femur (n=4 mice for each condition).

FIG. 8: Generation and validation of Hmgb1^{-/-} mice. (A) Schematic of generation and timeline for tamoxifen administration and determining mRNA expression and intracellular levels of HMGB1 in Hmgb1^{-/-} mice. (B, C) Skeletal, bone marrow, and muscle cells from Hmgb1^{-/-} mice show markedly reduced mRNA expression of HMGB1 (B), and intracellular levels of HMGB1 protein (C) compared to Hmgb1^{f1/f2} controls, as demonstrated by qRT-PCR and intracellular FACS staining respectively (n=4 mice for each condition). MFI=Median Fluorescence Intensity. (D) Schematic of determination of extracellular levels of HMGB1 post-fracture in Hmgb1^{-/-} mice. (E) Plasma levels of extracellular HMGB1^{f1/f1} are markedly lower in Hmgb1^{-/-} fractured mice compared to Hmgb1^{f1/f1} fractured mice, and equivalent to Hmgb1^{-/-} unfractured mice (n=4 mice for each condition).

FIG. 9: In vivo microCT radiographs of genetic ablation and pharmacological inhibition of HMGB1-CXCL12-CXCR4, time course of HMGB1-CXCL12 heterocomplex post-fracture, and mSSCs express functional CXCR4. (A) Hmgb1^{-/-} mice show markedly delayed fracture healing compared to Hmgb1^{f1/f1} controls, as shown by in vivo microCT radiographs (n=10 mice for each condition). (B) Circulating levels of HMGB1-CXCL12 heterocomplex in plasma after femoral fracture over 28 days. Plasma samples were collected at 1 h, 3 h, 6 h, 10 h, day 5, day 7 and day 28 post-fracture, and from unfractured mice (n=4 mice for each condition and time point). (C) Glycyrrhizin delays fracture healing compared to vehicle controls as shown by in vivo microCT radiographs (n=10 mice for each condition). (D) AMD3100 abrogates the effects of exogenous FR or 3S-HMGB1 as shown by in vivo microCT radiographs (n=10 mice for each condition). (E) mSSCs migrate to CXCL12, with a dose response, as determined by time lapse microscopy and measured by Euclidean distance (n=50 cells for each condition, similar results observed in 3 independent experiments).

FIG. 10: HMGB1 transitions murine and human stem cells to G_{Alert}, exogenous HMGB1 rescues the ATP G_{Alert} phenotype in Hmgb1^{-/-} mice, CXCL12 does not transition mSSCs to G_{Alert}, and stem cells remain in G_{Alert} 2 weeks following i.v. HMGB1 despite circulating levels of HMGB1 being at steady state levels at this time. (A) Rapamycin abrogates the effects of exogenous FR or 3S-HMGB1 as shown by microCT radiographs. (B) mHSCs and mMuSCs express CXCR4, as shown by FACS histogram plots (n=4 mice for each condition, with similar results observed in 3 independent experiments). (C, D) hHSPCs and hMSCs treated with FR or 3S-HMGB1 show elevated cellular ATP levels (C), and mitochondrial DNA (D) compared to vehicle controls, but much lower than IFN-γ or BMP2 activated cells respectively (n=4 HSPC donors, 4 hMSC donors, with similar results observed in 3 independent experiments). (E) mMuSCs from mice treated with a cMet inhibitor (PHA 665752) or anti-cMet, express substantially less surface CXCR4 compared to controls, as shown by FACS histogram plot, and quantified by MFI (n=4 mice for each condition). MFI=Median Fluorescence Intensity. (F) Hmgb1^{-/-} mice treated with FR or 3S-HMGB1 show elevated ATP levels for mSSCs, mHSCs and mMuSCs from contralateral limbs of fractured (#) mice (n=4 mice for each condition). (G) Systemic administration of CXCL12 does not lead to increased ATP levels in mSSCs compared to vehicle control (n=4 mice for each condition). (H) ATP levels of mSSCs, mHSCs, and mMuSCs remain elevated after 2 weeks following treatment with FR or 3S-HMGB1 (n=4 mice for each condition). (I) 2 weeks following i.v. FR or 3S-HMGB1 systemic HMGB1 levels are equivalent to steady state levels (n=6 steady state mice and 4 mice for each FR and 3S-HMGB1 conditions).

### DETAILED DESCRIPTION OF THE INVENTION

### Terms

As used herein, and unless stated otherwise, each of the following terms shall have the definition set forth below.

As used herein, including the appended claims, the singular forms of words such as “a,” “an,” and “the,” include their corresponding plural references unless the context clearly dictates otherwise.

As used herein, “effective” as in an amount effective to achieve an end means the quantity of a component that is sufficient to yield an indicated therapeutic response without undue adverse side effects (such as toxicity, irritation, or allergic response) commensurate with a reasonable benefit/risk ratio when used in the manner of this disclosure. For example, an amount effective to treat patient after fracture or other injury. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, an “amount” of a compound as measured in milligrams refers to the milligrams of compound present in a preparation, regardless of the form of the preparation. An “amount of compound which is 90 mg” means the amount of the compound in a preparation is 90 mg, regardless of the form of the preparation. Thus, when in the form with a carrier, the weight of the carrier necessary to provide a dose of 90 mg compound would be greater than 90 mg due to the presence of the carrier.

As used herein, “about” in the context of a numerical value or range means±10% of the numerical value or range recited or claimed.

As used herein, to “treat” or “treating” encompasses, e.g., inducing inhibition, regression, or stasis of the disorder and/or disease or promotion of repair and regeneration or recovery. As used herein, “inhibition” of disease progression or disease complication in a subject means preventing or reducing or reversing the disease progression and/or disease complication in the subject.

As used herein, “a biologically active truncated form of HMGB1” shall be understood to include all biologically active truncated forms of HMGB1 described in the prior art as of the filing date of this application.

The combination of the invention may be formulated for its simultaneous, separate or sequential administration, with at least a pharmaceutically acceptable carrier, additive, adjuvant or vehicle as described herein. Thus, the combination may be administered:
as a combination that is part of the same medicament formulation, the two active compounds are then administered simultaneously, or
as a combination of two units, each with one of the active substances giving rise to the possibility of simultaneous, sequential or separate administration.

As used herein, “concomitant administration” or administering “concomitantly” means the administration of two agents given in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding pridopidine therapy to a patient already receiving donepezil therapy.

The subject invention provides a method of preventing or treating a condition associated with a defect in, or damage to, an organ in a subject with, or at risk for, such defect or damage to such organ which comprises administering to the subject a therapeutically effective amount of the fully reduced form of HMGB1 or a biologically active truncated form of HMGB1 so as to prevent or treat such condition.

In one embodiment the method provides treatment of the condition. In another embodiment the method provides prevention of the condition.

In some embodiments the subject is anticipated to be in need of treatment of the condition at a point in the future.

In one embodiment the organ or its tissue relies on repair by stem or parenchymal cells that express the cell surface receptor CXCR4.

In one embodiment the organ is the brain, the spinal cord and/or associated nerves, peripheral nerves, blood vessels, an eye, the pancreas, the liver, a lung, the gut, or a kidney. In another embodiment the organ is the islets of Langerhans region of the pancreas. In a further embodiment the organ is the small intestine or the large intestine. Additionally, the organ may be the spleen, bladder, ureters, or a male or female reproductive organ or tract.

In one embodiment, the defect or damage to the organ is caused by acute injury, hemorrhage, occlusive stroke, Alzheimer's disease, or Parkinson's disease.

In an embodiment, the organ is the spinal cord and the defect or damage is caused by spinal cord trauma, motor neurone disease (MND), Amyotrophic Lateral Sclerosis (ALS), surgery for nerve root or cord decompression.

In another embodiment, the organ is the liver and the defect or damage is caused by chronic injury.

In some embodiments, the method further comprises promoting liver regeneration in patients with chronic injuries to the liver. The injury to the liver may occur after hepatitis C, alcoholic steatohepatitis or non-alcoholic steatohepatitis.

In one embodiment, the organ is a kidney and the patient is afflicted with renal disease. In an embodiment, the administration retards or stops progression of renal failure. In some embodiments, the renal disease is caused by trauma or a chronic kidney disease which causes scarring and/or fibrosis. In embodiments, the method further comprises healing the scarring and/or fibrosis.

In one embodiment, the organ is a lung and the patient is afflicted with lung disease. In an embodiment, the administration retards or stops progression of the lung disease. In some embodiments, the lung disease is idiopathic pulmonary fibrosis.

In one embodiment, the organ is the heart, the patient is afflicted with heart disease and the administration prevents progression to cardiac fibrosis and/or heart failure following injury. The injury may be, for example, myocardial infarct.

In one embodiment, the organ is skin and the defect or damage is surgical wounds. In some embodiments, the method reduces scarring following surgery. The surgery may be cosmetic surgery or other surgery.

In another embodiment, the organ is the gastrointestinal tract. In some embodiments, the defect or damage is caused by a surgery or a inflammatory bowel disease such as Crohn's disease or ulcerative colitis.

In one embodiment, the method further comprises administering the fully reduced form of HMGB1 in combination with other treatments, for example, a treatment to reduce the defect or damage while the fully reduced (all thiol) form of HMGB1 promotes repair and regeneration of the defect or damage.

In some embodiments the subject is in need of treatment of the condition presently.

The subject invention also provides a method of improving regeneration of blood in a subject comprising administering a therapeutically effective amount of the fully reduced form of HMGB1 or a biologically active truncated form of HMGB1, effective to improve regeneration of blood.

In one embodiment the fully reduced form of HMGB1, or the biologically active truncated form of HMGB1 is effective to improve regeneration of blood in the subject.

In one embodiment the subject is anticipated to be in need of improved regeneration of blood at a point in the future. In another embodiment the subject is in need of improved regeneration of blood presently.

In one embodiment, the subject is afflicted with Alzheimer's Disease, Amyotrophic Lateral Sclerosis (Motor Neuron Disease) or Parkinson's Disease. In another embodiment the subject is affected by or at risk for stroke.

In one embodiment, the administration is systemic. In another embodiment, the administration is local.

In some embodiments, the administration is into the cerebrospinal fluid (intrathecal). In other embodiments, the administration is topical, for example, around nerves, tendons, and/or bones.

In one embodiment, the condition is tissue damage or tissue loss, or blood damage or blood loss.

In some embodiments, the fully reduced form of HMGB1 is administered to the subject. In other embodiments the biologically active truncated form of HMGB1 is administered to the subject.

In one embodiment the fully reduced form of HMGB1, or the biologically active truncated form of HMGB1 is a fully reduced (FR) all-thiol HMGB1 (FR-HMGB1).

In another embodiment the fully reduced form of HMGB1 or the biologically active truncated form of HMGB1 is a recombinant non-oxidable one-serine form (1S) of HMGB1 (1S-HMGB1) in which a cysteine at one of C23, C45, or C106 is replaced by a serine.

In a further embodiment the fully reduced form of HMGB1 or the biologically active truncated form of HMGB1 is a recombinant non-oxidable two-serine form (2S) of HMGB1 (2S-HMGB1) in which the cysteines at both C23 and C45 or both C45 and C106 are replaced by a serine.

In some embodiments, the fully reduced form of HMGB1, or the biologically active truncated form of HMGB1 is a recombinant non-oxidable all-serine form (3S) of HMGB1 (3S-HMGB1) in which the cysteines at each of C23, C45, and C106 are replaced by a serine.

In one embodiment the administration of the fully reduced form of HMGB1 or the biologically active truncated form of HMGB1 is one day to one month prior to said point in the future.

In some embodiments, the method comprises administering the fully reduced form of HMGB1 or the biologically active truncated form of HMGB1 after injury, preferably if the patient is afflicted with an acute injury. In other embodiments, the method comprises administering the fully reduced form of HMGB1 or the biologically active truncated form of HMGB1 intermittently after the initial administration, preferably if the patient is afflicted with a chronic disorder.

The methods of the present invention may be used in combination therapy, which includes simultaneous, separate sequential or concomitant administration and add-on therapy. For example, the subject invention provides a method of administering a pharmaceutical composition capable of reducing tissue damage and administering a fully reduced form of HMGB1 or a biologically active truncated form of HMGB1. Preferably, the administration is concomitant administration. Such combination therapy is especially pertinent for treating the liver (for example, in patients afflicted with non-alcoholic or alcoholic steatohepatitis), pancreatic islet cells (for example in patients afflicted with type I diabetes), and neurodegenerative disorders. Combination therapy including the methods of the present invention may also be used to treat other tissues, such as the lung, kidney, gut, muscle (skeletal or cardiac), skin and bones.

In some embodiments, the method further comprises administering a pharmaceutical composition capable of reducing liver inflammation or fibrosis. In other embodiments, the method further comprises administering a pharmaceutical composition capable of reducing lung inflammation or fibrosis. In additional embodiments, the method further comprises administering a pharmaceutical composition capable of reducing kidney inflammation or fibrosis.

In some embodiments, the method further comprises administering a pharmaceutical composition capable of reducing damage to pancreatic islet cells, for example, in patients afflicted with type I diabetes. In other embodiments, the method further comprises administering a pharmaceutical composition capable of reducing damage to gut cells.

In some embodiments, the method further comprises administering a pharmaceutical composition capable of treating a neurodegenerative disorder preferably selected from the group consisting of: Amyotrophic Lateral Sclerosis (ALS), Alzheimer's disease and Parkinson's disease. The subject may be afflicted with Amyotrophic Lateral Sclerosis (ALS), Alzheimer's disease or Parkinson's disease.

Data has shown that remaining in Gₐₗₑᵣₜ over prolonged periods leads to stem cell exhaustion and depletion. According, patients are given a recovery time between doses. In some embodiments, the recovery time is 1 year. The recovery time may also be 1 month, 3 months, or 6 months.

In patients with severe acute injuries, for example liver after drug overdose/poisoning or multiple trauma patients with an injury severity score of greater than 15 the initial massive injury leads to high levels of endogenous HMGB1 followed by high levels of local and potentially also systemic inflammatory response. In these patients, therapeutic administration of the fully reduced form of HMGB1 would be delayed until after the inflammation has subsided and as the patient/organ enters the reparative phase.

This invention will be better understood by reference to the Experimental Details which follow, but those skilled in the art will readily appreciate that the specific experiments detailed are only illustrative of the invention as described more fully in the claims which follow thereafter.

### EXAMPLES

Alarmins are a group of evolutionarily unrelated endogenous molecules with diverse homeostatic intracellular roles, which when released from dying, injured or activated cells trigger an immune/inflammatory response (Harry 2008, Glass 2011, and Chan 2015). Much effort has been focused on their deleterious role in autoimmune and inflammatory conditions (Chan 2015, Scaffidi 2002, Terrando 2010, Harris 2012, and Horiuchi 2017). Of the few studies (Chan 2012, Tirone 2018) that have investigated the role of alarmins in tissue repair, none have used a combination of human tissues and multiple animal injury models to characterize their effects on precise flow cytometry-defined endogenous adult stem cells in vivo. In the following examples, it has been demonstrated that High Mobility Group Box 1 (HMGB1) is a key upstream mediator of tissue regeneration which acts by transitioning CXCR4+ skeletal, hematopoietic and muscle stem cells from Gₒ to G_{Alert}.

The following Examples also demonstrate that, in the presence of appropriate activating factors, exogenous administration before or at the time of injury leads to accelerated tissue repair.

### Example 1

### Materials and Methods:

The objective of this study was to understand the role of alarmins in tissue regeneration in vivo through their effects on adult stem cells, and the translational relevance of these findings. We used human samples and primary human cells and multiple murine models of injury and regeneration. For prospective multi-parameter flow cytometry assays, we used well-established skeletal, hematopoietic and muscle stem cell-surface markers, and published isolation protocols (Chan 2015, Wilson 2008, Liu 2015). Sample size (n values) are reported as biological replicates of human donors and mice. The magnitude of the effect and variability in the measurements were used to determine sample size and replication of data. Although samples were not specifically randomized or blinded, mouse identification numbers were used when possible as sample identifiers. Therefore, the genotypes and experimental conditions of each mouse/sample were not readily known to the experimenters during sample processing and data collection. Animals were excluded from the study only if their health status was compromised.

Human and murine plasma: Plasma samples from patients who had sustained femoral fractures and from healthy unfractured controls were obtained from the John Radcliffe Hospital (REC: 16/SW/0263, PID: 12229, IRAS: 213014). The human plasma samples were from the patient's first in-hospital blood sampling, typically within 4 hours post-fracture. Murine plasma was collected 3 hours post-femoral fracture via cardiac puncture from 12 week old female C57B16/J wild type, Hmgb1^{f1/f1}, Hmgb1^{-/-} mice, and from healthy unfractured controls. For the circulating levels of HMGB1, S100A8/A9 and HMGB1-CXCL12 heterocomplex over a 4 week period, murine plasma samples were collected from 12 week old female C57B16/J wild type at 1 hour, 3 hours, 6 hours, 10 hours, 5 days, 7 days, and 28 days after fracture injury. To assess the induction of inflammation-related cytokines by HMGB1, plasma samples were collected via cardiac puncture from 12 week old female C57B16/J wild type mice at 0.5 hours, 1 hour, 3 hours, 18 hours, 48 hours, and 2 weeks post intravenous (i.v.) administration of 0.75 mg/kg of FR, or 3S-HMGB1. Samples were collected at 3 hours post i.v. administration of 0.75 mg/kg of DS-HMGB1, or 0.5 μg/kg of LPS. All human and murine samples were aliquoted, frozen, and stored at −80° C. before being thawed and assayed.

Mice: All animal procedures were approved by the institutional ethics committee and the United Kingdom Home Office (PLL 71/7161, and PLL 30/3330), and were performed on skeletally mature 12-14 week old female C57BL/6J (Charles River), and transgenic mice. Hmgb1^{-/-} mice were generated by crossing Hmgb1^{f1/f1} (Riken) with Rosa-CreER^{T2} mice (Jackson Laboratory), and at 10 weeks of age administering 3 intraperitoneal (i.p.) injections of 1.5 mg tamoxifen (Sigma) on alternate days over a 6 day period, in a mixture of sunflower seed oil (Sigma) and 10′ ethanol (VWR). Mice were used 7 days after the last tamoxifen injection. Hmgb1^{-/-} mice were obtained at the expected Mendelian ratio with no adverse phenotypic side effects, and Hmgb1^{f1/f1} mice (not crossed with Rosa Cre-ER^{T2+/+} mice) treated with tamoxifen were used as controls. Animals were genotyped by PCR of earclip DNA, with the primer sequences in Table 1 below, using the HotStart Mouse Genotyping Kit (Kapa Biosystems).

TABLE 1
Primers for genotyping of
mice and q-PCR experiments

| Primer | Hmgb1 [symbol] | Primer | Rosa-CreER^{T2} |
|---|---|---|---|
| HMGB1 | TGTCATGCCACCCT | Common | AAGGGAGCTGCAGT |
| Forward | GAGCAGTT | Forward | GGAGTA |
| | (SEQ ID NO: 1) | | (SEQ ID NO: 3) |
| HMGB1 | TGTGCTCCTCCCGG | Wild | CCGAAAATCTGTGG |
| Reverse | CAAGTT | Type | GAAGTC |
| | (SEQ ID NO: 2) | Reverse | (SEQ ID NO: 4) |
| | | Mutant | CGGTTATTCAACTT |
| | | Reverse | GCACCA |
| | | | (SEQ ID NO: 5) |

[symbol]

indicates data missing or illegible when filed

### Fracture Model

Animals were anesthetized by aerosolized 2% isoflurane, given analgesia and transferred to a warming pad. The right upper hind limb was shaved and skin prepared with povidone iodine solution. After incising the skin, the femur was exposed by blunt dissecting through the fascia lata between the biceps femoris and gluteus superficialis muscles. A commercial external fixator jig was fitted (RISystem) and a 0.5 mm osteotomy created in the femoral diaphysis with a Gigli wire. The wound was closed with interrupted non-absorbable 6/0 Prolene sutures (Ethicon). Immediately postoperatively all mice were given subcutaneous hydration, analgesia and allowed to mobilize freely. Postoperative analgesia continued for 2 days. Mice were treated locally at the time of injury with an injection into the fascial pocket surrounding the osteotomy of 0.75 mg/kg FR-HMGB1 (HMGBiotech), 0.075 mg/kg, 0.75 mg/kg, or 7.5 mg/kg 3S-HMGB1 (HMGBiotech), 0.075 mg/kg CXCL12 (R&D), or 50 μl PBS vehicle control; 50 mg/kg glycyrrhizin (Sigma), or 50 μl DMSO:PBS 1:1 vehicle control; 3 mg/kg AMD3100 (Abcam), or 50 μl PBS vehicle control; 4 mg/kg rapamycin (LC Laboratories), or 50 μl DMSO:PBS 1:1 vehicle control. Glycyrrhizin was used to disrupt the formation of the HMGB1-CXCL12 heterocomplex as it is the only known specific inhibitor for blocking the binding site of CXCL12 on HMGB1 (Schiraldi 2012, Mollica 2007). Antibodies to HMGB1 do not specifically block the interaction with CXCL12 and may have other off target effects. AMD3100 was used to disrupt the binding of CXCL12 to CXCR4 as it is a specific and clinically approved inhibitor of the CXCL12-CXCR4 interaction. It was used to determine the receptor through which the HMGB1-CXCL12 heterocomplex acted, using the rate of fracture healing as a measure of this interaction. AMD3100 or other inhibitors, such as anti-CXCL12, of the CXCL12-CXCR4 axis for cellular level characterizations of the G_{Alert} state were not used as this would have resulted in activation and release of stem cells from their niche, CXCL12-CXCR4 signaling being well known for enforcing the quiescent G₀ state (Peled 1999, Sugiyama 2006, Nie 2008, Tzeng 2011, Ding 2013, Greenbaum 2013). For priming experiments, mice were treated systemically 2 weeks prior to injury with an i.v. injection of 0.75 mg/kg FR-HMGB1, 0.75 mg/kg 3S-HMGB1, or 50 μl PBS vehicle control.

Cytokine analysis: Enzyme-linked immunosorbent assays (ELISAs) were used to measure levels of TNF, S100A8/A9 (R&D), HMGB1 (IBL International), and HMGB1-CXCL12 heterocomplex (R&D; IBL International) in human monocyte supernatant, and human and murine plasma samples. These were ‘sandwich’ ELISAs where the antigen of interest was quantified between two layers of antibodies: the capture and the detection antibody. For S100A8/A9 and HMGB1, commercial kits were used according to manufacturer's instructions. For HMGB1-CXCL12, we used the heterocomplex hybrid ELISA (Venereau 2012, Schiraldi 2012). The reagents for the TNF and HMGB1-CXCL12 ELISA are listed in Table 2 below. Further immunoassays to quantify circulating levels of inflammation-related cytokines, TNF, IL-6, and IL-10, in mouse plasma following i.v. administration of FR, 3S or DS-HMGB1, or LPS were performed using commercial kits based on electrochemiluminescense (MesoScale Discovery) as per manufacturer's instructions.

TABLE 2
Reagents for TNF and HMGB1-CXCL12 hybrid ELISAs.

| ELISA | TNF | HMGB1-CXCL12 heterocomplex |
|---|---|---|
| Capture Antibody | Mouse anti-human TNF | Mouse anti-human CXCL12 |
| | (BD Bioscience, 551220) | (R&D, DY350) |
| Protein Standard | Human TNF | FR-HMGB1 (HMGBiotech, HM-114) and |
| | (R&D, 210-TA) | human CXCL12 (R&D, 350-NS-050), in a 1:2 ratio. |
| Detect Antibody | Biotin mouse anti-human TNF | anti-HMGB1 |
| | (BD Bioscience, 554511) | (IBL International, ST51011) |
| Substrate | Streptavidin-HRP (R&D, DY998), | Substrate A and Substrate B |
| | TMB Microwell Peroxidase Substrate Kit (KPL, 507603) | (IBL International, ST51011) |
| Stop | Sulfuric Acid | Stop Solution |
| | (Sigma, 320501) | (IBL International, ST51011) |
| Read | Mithras Multimode Microplate Reader LB 940 | Mithras Multimode Microplate Reader LB 940 |
| | (Berthold Technologies) 450 nm | (Berthold Technologies) 450 nm |

### Human MSC Osteogenesis Screen

Human MSCs (Lonza) were maintained in DMEM (Gibco), supplemented with 10% FBS (Gibco), 1 L-Glutamine (GE), and 1% penicillin/streptomycin (GE), in standard tissue culture conditions (37° C.; 5% CO₂), and used between passages 3-5. Human monocytes were isolated from human peripheral blood leucocyte cones (John Radcliffe Hospital, NHS Blood and Transplant) by positive selection with CD14 MACS microbeads (Miltenyi Biotech) and an autoMACS machine. To determine the direct effect of the alarmins, S100A8, S100A9 (supplied by T.Vogl, Munster), FR-HMGB1, DS-HMGB1, and 3S-HMGB1 (HMGBiotech), or LPS (ALEXIS Biochemicals), on hMSC osteogenesis, 10⁴ hMSCs were plated in triplicate into wells of a 96 well plate with various concentration of alarmins, or LPS, in 200 μl of osteogenic media. The latter consisted of maintenance media supplemented with 100 nM dexamethasone (Sigma), 50 μg/ml ascorbic acid 2-phosphate (Sigma), and 10 mM s-glycerophosphate (Sigma). Treatment with oncostatin M 10 ng/ml (Peprotech) was used as a positive control. To determine the effects of alarmins on hMSC osteogenesis in the presence of monocytes or their products, monocytes were co-cultured with hMSCs in a ratio of 10:1 (10, monocytes: 10⁴ hMSCs) in osteogenic media with various concentrations of alarmins or LPS; or monocytes were incubated with various concentrations of alarmins or LPS, for 16 hours and the resulting supernatant was subsequently applied onto hMSCs. To determine the effects of priming hMSC with alarmins, hMSCs were plated in maintenance media with various concentrations of alarmins; after 16 hours this was changed to osteogenic media alone. For all permutations, the respective media was replaced at day 3, and at day 7 the media was removed, cells lysed in 20 μl NP-40 lysis buffer, and alkaline phosphatase (ALP) activity, which is a marker of osteogenic differentiation, was quantified using a commercial kit (WAKO Chemicals) as per manufacturer's instructions.

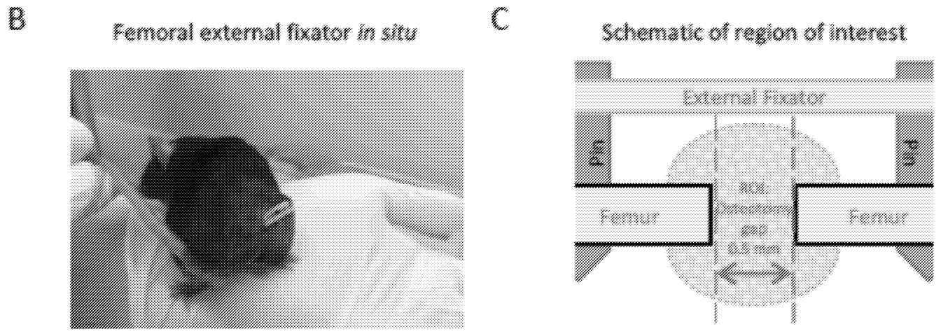

In vivo micro computed tomography (CT) setup and analyses: In routine orthopaedic practice, and in clinical trials, longitudinal radiographic investigations are the most widely used tool for assessing the progression of fracture healing. Therefore, similar assessment of murine models of fracture healing would have increased translational relevance. Radiographic assessments of bone tissue are also well-known to correlate highly with histological findings (Gregor 2012, Particelli 2012), and have the added advantage of being non-destructive, thereby allowing longitudinal assessment of each animal. MicroCT imaging was performed using a high-speed rotating gantry based system (PerkinElmer, Quantum FX). Animals were anaesthetised briefly with aerosolised isofluorane 2 for each 3 minute scan. The X-ray source was set to a current of 200 μA, voltage of 90 kVp, and a field of view of 5 mm to encompass the two fixator pins closest to the osteotomy gap, for a voxel resolution of 10 μm. After the scans, mice were revived in a heated box and returned to their cages. Scans were analyzed using a commercially available microCT software package Analyze12 (AnalyzeDirect), which permitted co-registration of scans acquired over a time course. The region of interest was defined as the bridging callus, which included only the tissue that formed in the osteotomy gap (FIG. 7C). Global thresholding (O'Neill 2012) was performed to distinguish between mineralized (hard callus), poorly mineralized (soft callus) and non-mineralised (fibrous) tissue. Callus volume included the volume of both hard and soft callus. Callus bone mineral density was the density of hard mineralised tissue, otherwise previously known as tissue mineral density (O'Neill 2012), and was calibrated by means of phantoms with known densities of calcium hydroxyapatite.

Mechanical strength testing: Mechanical strength testing is a well-established functional measure of callus/bone strength and fracture healing. Three-point bend testing was used as it is a well-established, reproducible and robust procedure for assessing the mechanical strength of the fracture callus, and is superior to other techniques such as axial loading testing (Steiner 2015). Both hind limbs were harvested after the final microCT scan, immediately dehydrated and fixed in 70% ethanol for at least 24 hours. Prior to three-point bend testing (FIG. 7F), all soft tissues overlying the femurs and the external fixator were removed, and the clean femurs were rehydrated in PBS for 3 hours at room temperature. The load cell was applied directly onto the callus, preloaded to a minimum of 0.03 N with the assistance of specimen protection and re-zeroed. Load was applied at a rate of 1 mm/minute until failure, and force-extension profiles were recorded. The resulting data were analysed using the BlueHill 3 (Instron) software package and the maximum force prior to fracture (FIG. 7G) of the injured femur was compared to the contralateral uninjured femur.

Isolation of stem cells: BD LSRFortessa X-20 and BD FACSAria III were used for flow cytometry and fluorescence activated cell sorting (FACS) respectively. Subsequent data analyses were performed with the FlowJo V10 software (TreeStar). Murine skeletal, muscle, and haematopoietic stem cells were defined and freshly isolated according to previously reported protocols (Chan 2015, Wilson 2008, Liu 2015). Bone, bone marrow, and muscle cell suspensions were created by respectively crushing femurs and enzymatically digesting with collagenase 800 U/ml (Worthington-Biochem), or extracting bone marrow plugs by flushing femurs with FACS buffer (Miltenyi Biotec) using a 25 gauge needle, or mincing thigh muscles and enzymatically digesting with collagenase 800 U/ml and dispase 1 U/ml (Gibco). Bone and bone marrow cell suspensions were also enriched by treatment for 5 minutes with red blood cell lysis buffer (Sigma). Thereafter all suspensions were strained through 70 μm and 40 μm filters (Greiner Bio-One) and stained with respective antibodies. Definitions were: mSSC, CD45⁻Ter119⁻Tie2⁻ AlphaV⁺Thy⁻6C3⁻CD105⁻CD200⁺; mMuSC, CD31⁻CD45⁻Sca-1⁻VCAM1⁺; mHSC, Lineage⁻ (CD2⁻CD3⁻CD4⁻CD5⁻CD8⁻CD11a⁻CD11b⁻TER119⁻B220⁻Gr-1⁻) c-Kit⁺Sca- 1⁺CD34⁻CD48⁻CD150⁺. Antibodies were: mSSC, CD45 (30-F11, BD), TER-119 (TER-119, BD), Tie2 [CD202b] (TEK4, Biolegend), AlphaV [CD51] (RMV-7, Biolegend), Thy1.1 [CD90.1] (OX-7, Biolegend), Thy1.2 [CD90.2] (30-H12, Biolegend), 6C3 [Ly-51] (6C3, Biolegend), CD105 (MJ7/18, Biolegend), CD200 (OX-90, BD); mMuSC, CD31 (MEC13.3, Biolegend), CD45 (30-F11, Biolegend), Sca-1 (D7, Biolegend), VCAM [CD106] (429, Biolegend); HSC CD2 (RM2-5, Biolegend), CD3 (17A2, Biolegend), CD4 (RM4-5, Biolegend), CD5 (53-7.3, Biolegend), CD8 (53-6.7, Biolegend), CD11a (M17/4, Biolegend), CD11b (M1-70, Biolegend), B220 [CD45R] (RA3-6B2, Biolegend), Gr-1 (RB6-8C5, Biolegend), TER-119 (TER-119, Biolegend), c-Kit [CD117] (2B8, Biolegend), Sca-1 (D7, Biolegend), CD34 (HM34, Biolegend), CD48 (HM48-1, Biolegend), CD150 (TC15-12F12.2, Biolegend). Stem cells were also stained for the presence of surface CXCR4 (2B11, BD), and intracellular HMGB1 (3E8, Biolegend). Human CD34+ haematopoietic stem and progenitor cells were isolated from human peripheral blood leucocyte cones (John Radcliffe Hospital, NHS Blood and Transplant) by magnetically activated cell sorting (MACS)(Peytour 2010) using the CD34 MicroBead Kit (Miltenyi Biotech) and an autoMACS machine.

Quantitative real-time PCR (qRT-PCR): Total RNA was isolated using TR1 reagent (Zymo Research) from cells from whole bone, bone marrow, and muscle cell suspensions using Direct-Zol™ RNA MiniPrep (Zymo Research) as per manufacturer's instructions. HMGB1 gene expression was determined by qRT-PCR and normalised to Gapdh. The amplifying primers were as follows, Gapdh (TaqMan, Mouse: Mm99999915_g1 Gapdh) and Hmgb1 (TaqMan, Mouse: Mm00849805_gH Hmgb1). All reactions were performed in an ViiA7 Real Time PCR System (Applied Biosystems) using TaqMan Fast Advanced MasterMix (Applied Biosystems) according to the manufacturer's instructions.

Cell cycle kinetics: To evaluate cell cycle propensity, pulse labelling with BrdU (Abcam) was performed with animals injected with 10 mg of BrdU i.p. 10 hours before cell isolation from whole femurs. Mice were treated locally at the time of fracture with 15 mg/kg FR-HMGB1, 15 mg/kg 3S-HMGB1, 15 mg/kg BMP2 (Peprotech), or 50 μl of PBS vehicle control. To evaluate speed of entry to cell cycle, continuous labelling with BrdU was performed by administering 6.5 mg/ml in their drinking water with 5% sucrose for the indicated period. BrdU incorporation was quantified with the commercially available BrdU FlowKit (BD) as per manufacturer's instructions. Following cell isolation and staining, cells were fixed and permeabilized with Cytofix/Cytoperm (BD) for 15 minutes at room temperature, buffered with Permeabilization Buffer Plus (BD) for 10 minutes at 4° C., re-fixed with Cytofix/Cytoperm for 5 minutes at room temperature, then treated with 30 μg/ml DNase (BD) for 1 hour at 37° C. to expose incorporated BrdU, and lastly stained with anti-BrdU (BD). Mice were treated systemically at the initiation of continuous BrdU administration with an i.v. injection of 15 mg/kg FR-HMGB1, 15 mg/kg 3S-HMGB1, or 100 μl of PBS vehicle control. The cells from these mice were compared to cells from the fractured side of injured mice who had also been administered continuous BrdU.

Cell migration: In vivo cell migration to the fracture site was determined by quantifying the number of BrdU⁻ cells in fractured femurs 12 hours post-fracture using flow cytometry and Precision Count Beads (Biolegend). Mice were administered 10 mg of BrdU i.p. at the time of fracture and treated locally with 0.075 mg/kg CXCL12 or 50 μl PBS vehicle. Subsequently, BrdU incorporation in the bone and bone marrow cell suspensions from the fractured femurs was determined using the commercially available BrdU FlowKit (BD) as per manufacturer's instructions.

In vitro cell migration of mSSCs was determined by placing 1000 freshly FACS isolated mSSCs in 6 μl of DMEM in the middle observation channel of collagen coated μ-Slide Chemotaxis slides (Ibidi). A chemotactic gradient was established across the observation channel by pipetting 70 μl DMEM 0? FBS into the left reservoir, and into the right reservoir either 0.15 μg/ml or 1.5 μg/ml CXCL12, or 0% or 20% FBS controls. The channels and reservoirs were plugged to prevent evaporation and cell migration was followed by time-lapse microscopy using an automated xyz motorized stage (Prior Scientific, Prior Proscan II), a climate chamber at 37° C., 5% CO₂, with humidity (Solent Scientific), a spinning disk Nikon Eclipse TE2000-U microscope with a 10× objective, and Volocity 6.3 (PerkinElmer) recording software. Cells were monitored over a period of 22 hours by capture of brightfield images every 5 minutes. Migration of 50 cells was analyzed using the automatic tracking function within the Imaris 6.7 (Bitplane) software, and represented using the Chemotaxis and Migration Tool 2.0 (Ibidi). Cells were excluded if track length was less than 50 μm.

Mitochondrial DNA: DNA was extracted from 1000 freshly FACS isolated mSSCs, mMuSCs, and mHSCs, and from 10000 trypsinised hMSCs, and 10000 MACS isolated hHSPCs, using the QIAamp DNA Micro Kit (Qiagen) as per manufacturer's instructions. mtDNA was quantified by qRT-PCR using primers amplifying the Cytochrome B region on mtDNA (TaqMan, Mouse: Mm04225271_g1 CYTB; Human: Hs 02596867_s1 MT_CYB) relative to the β-globin region on gDNA (Taqman, Mouse: Mm 01611268_g1 Hbb-b1; Human: 00758889_s1 HBB). Mice were treated systemically with an i.v. injection of 0.75 mg/kg FR-HMGB1, 0.75 mg/kg 3S-HMGB1, or 100 μl of PBS vehicle control. The cells from these mice were compared to cells from the uninjured contralateral side of fractured animals. hMSCs were treated for 16 hours with 10 μg/ml FR-HMGB1 in DMEM, 10 μg/ml 3S-HMGB1 in DMEM, DMEM vehicle control, or osteogenic media supplemented with 10 μg/ml BMP2. Whole human peripheral blood leucocyte cones were treated for 2 hours with 1.5 μg/ml FR-HMGB1, 1.5 μg/ml 3S-HMGB1, 10 ng/ml IFN-γ (Miltenyi Biotec), or RPMI (Lonza) vehicle control.

Cellular ATP: Cellular ATP levels of 1000 freshly FACS isolated mSSCs, mMuSCs, and mHSCs, and from 10000 trypsinised hMSCs, and 10000 MACS isolated hHSPCs, were quantified using the commercially available ATP Bioluminescence Assay Kit CLS II (Roche), and used as per manufacturer's instructions. Cells were pelleted, boiled in 100 mM Tris, 4 mM EDTA, pH 7.75 for 2 minutes, pelleted again, and luciferase reagent was added to the supernatant. This was then read on a FLUOstar Omega (BMG Labtech) spectrophotometer, with the luminescence optic. Mice were treated systemically with an i.v. injection of 0.75 mg/kg FR-HMGB1, 0.75 mg/kg 3S-HMGB1, 0.075 mg/kg CXCL12 or 100 μl of PBS vehicle control. The cells from these mice were compared to cells from the uninjured contralateral side of fractured animals. hMSCs were treated for 16 hours with 10 μg/ml FR-HMGB1 in DMEM, 10 μg/ml 3S-HMGB1 in DMEM, DMEM vehicle control, or osteogenic media supplemented with 10 μg/ml BMP2. Whole human peripheral blood leucocyte cones were treated for 2 hours with 1.5 μg/ml FR-HMGB1, 1.5 μg/ml 3S-HMGB1, 10 ng/ml IFN-γ (Miltenyi Biotec), or RPMI (Lonza) vehicle control.

Cell size: Freshly FACS isolated mSSCs, mMuSCs, and mHSCs, trypsinised hMSCs, and MACS isolated hHSPCs, were placed onto a haemocytometer and stained with 0.4% trypan blue solution (Sigma). Bright field images of the haemocytometer were acquired with an Olympus CKX41 microscope using a 40× objective lens. The analysis of cell diameter was manually performed using the Fiji distribution of ImageJ2 software (NIH) (Schindelin 2012). Mice were treated systemically with an i.v. injection of 0.75 mg/kg FR-HMGB1, 0.75 mg/kg 3S-HMGB1, or 100 μl of PBS vehicle control. The cells from these mice were compared to cells from the uninjured contralateral side of fractured animals. cMet inhibition: Mice were treated i.p. twice a day for 5 consecutive days with 7.5 mg/kg of the c-Met inhibitor PHA 665752 (Selleckchem), or 7.5 μl DMSO in 400 μl of PBS vehicle control, or they were treated i.p. once a day for 2 consecutive days with 0.5 mg/kg anti-cMet (R&D), or 0.5 mg/kg goat IgG isotype control (R&D) in 400 μl of PBS. Following the treatment period mice were sacrificed, mMuSCs isolated, and stained for CXCR4 surface expression.

Haematological injury model: Animals were warmed up in a heating box, transferred to a restraining device, and a single i.v. injection of 150 mg/kg 5-fluorouracil (Sigma) was administered via the tail vein. 40 μl of peripheral blood was collected at the times indicated from the tail vein with EDTA-containing Microvettes (Sarstedt). 10 μl of this sample was smeared onto slides, air-dried, stained with Giemsa (Sigma) and May Grunwald solutions (RA Lamb), and neutrophils and leucocytes were counted with light microscopy using an Olympus BX51 microscope and a 40× objective lens to determine the differential neutrophil count. The remainder of the sample was treated for 5 minutes with red blood cell lysis buffer (Sigma), stained with 0.4% trypan blue solution (Sigma), and leucocytes were counted with a haemocytometer to quantify total peripheral leucocytes. Together with the differential neutrophil count as above, the total neutrophil count was also determined. Mice were treated systemically at the time of injury or systemically 2 weeks prior to injury with an i.v. injection of 0.75 mg/kg FR-HMGB1, 0.75 mg/kg 3S-HMGB1, or 100 μl of PBS vehicle control.

Muscle injury model: Animals were anesthetized by aerosolised 2% isoflurane, given analgesia, transferred to a warming pad and the right lower hindlimb was shaved and skin was prepared with povidine iodine. 80 μl of 1.2% BaCl₂ (Sigma) was injected into and along the length of the tibialis anterior (TA) muscle (Rodgers 2014). Immediately postoperatively all mice were given analgesia and allowed to mobilize freely, and given postoperative analgesia for 2 days. Mice were euthanized and TA muscles extracted at the times indicated, fixed in 4% paraformaldehyde (Santa Cruz Biotechnology) for 24 hours, embedded in paraffin, sectioned, stained with haematoxylin and eosin to identify centrally nucleated fibres, and imaged with an Olympus BX51 using a 40× objective lens. The cross-sectional area (CSA) of the fibres that were approximately midway along the proximal-distal axis of the TA muscle belly was manually measured using the Fiji distribution of ImageJ2 software (NIH) (Schindelin 2012). Mice were injected intramuscularly at the time of injury or intravenously 2 weeks prior to injury, with 0.75 mg/kg FR-HMGB1, 0.75 mg/kg 3S-HMGB1, or 50 μl or 100 μl of PBS vehicle control respectively.

Statistical analysis: Statistical analyses were performed using GraphPad Prism 7 (GraphPad Software). Unless stated otherwise, significance was calculated using two-tailed unpaired Student's t-tests. For microCT callus volumes, bone mineral density, and in vivo cycling to continuous BrdU administration, significance was calculated using non-linear curve fitting and the F-test (FIGS. 7 D and E). All results are shown as mean±SD, except for curve fitting results which are shown as mean±95% CI. Results were considered statistically significant when p<0.05. Significant results were expressed using asterisks, where *p<0.05, **p<0.01, ***p<0.001, ****p<0.0001. This convention was used throughout.

### Results

### Alarmins are Elevated Post-Injury in Humans and Mice.

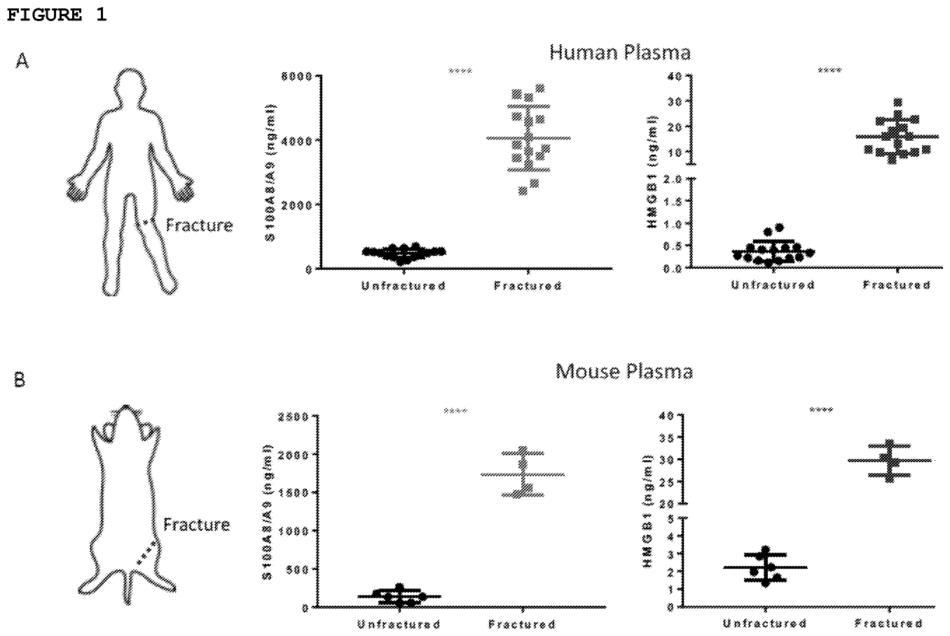

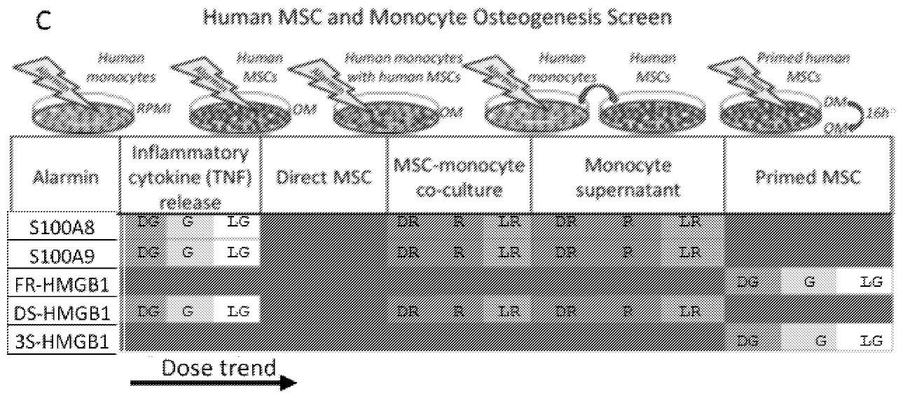

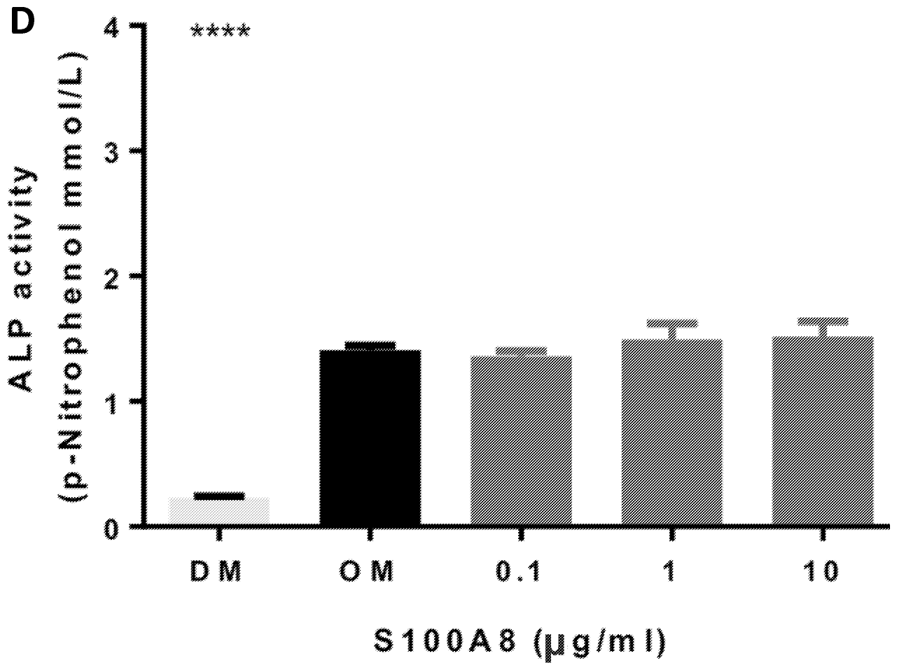

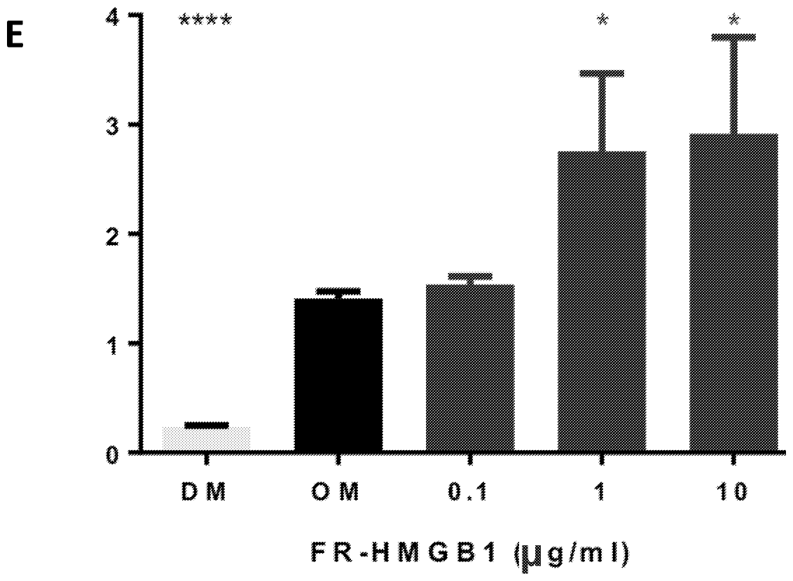

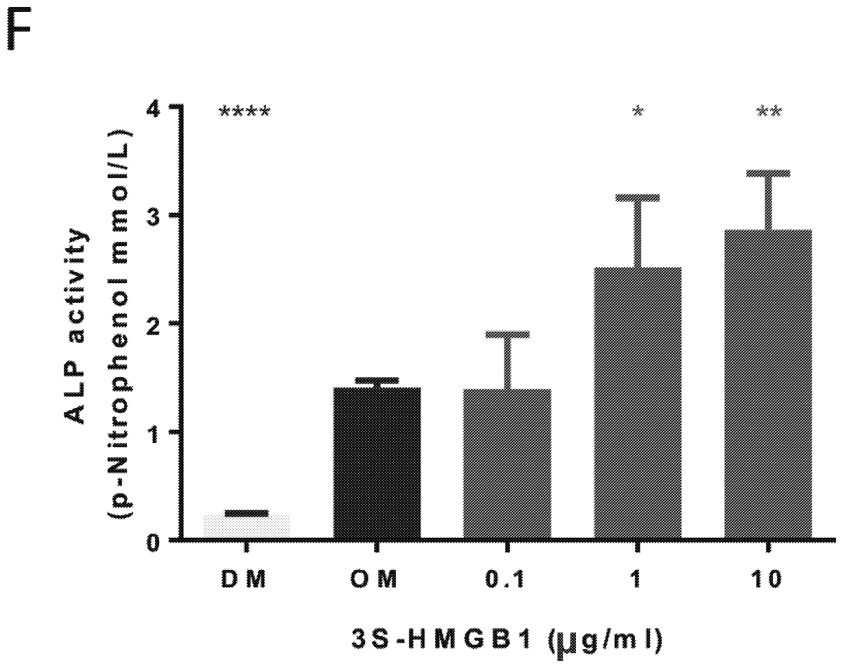

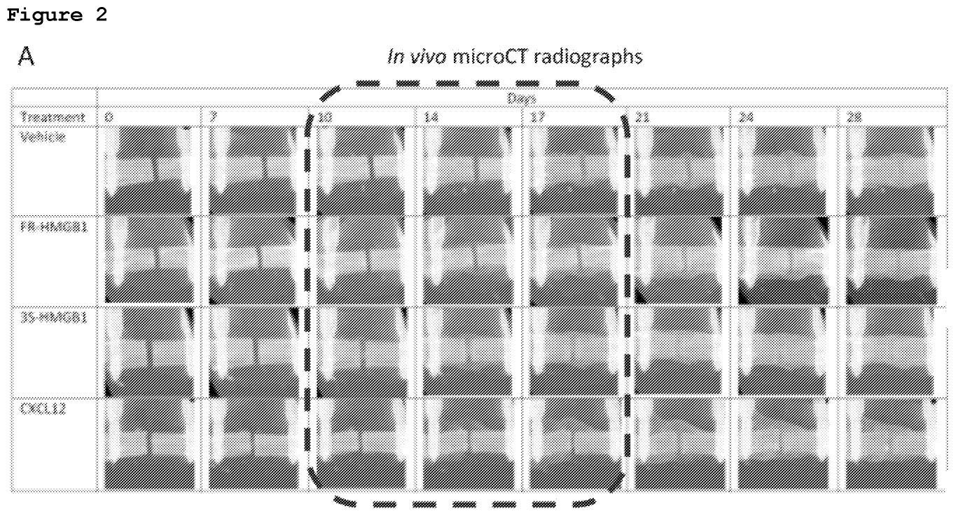

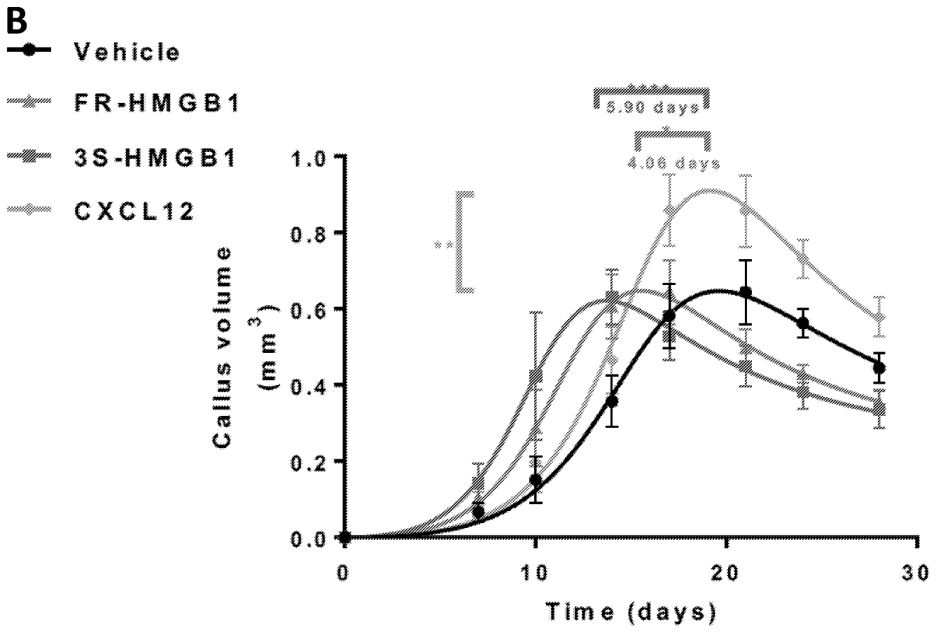

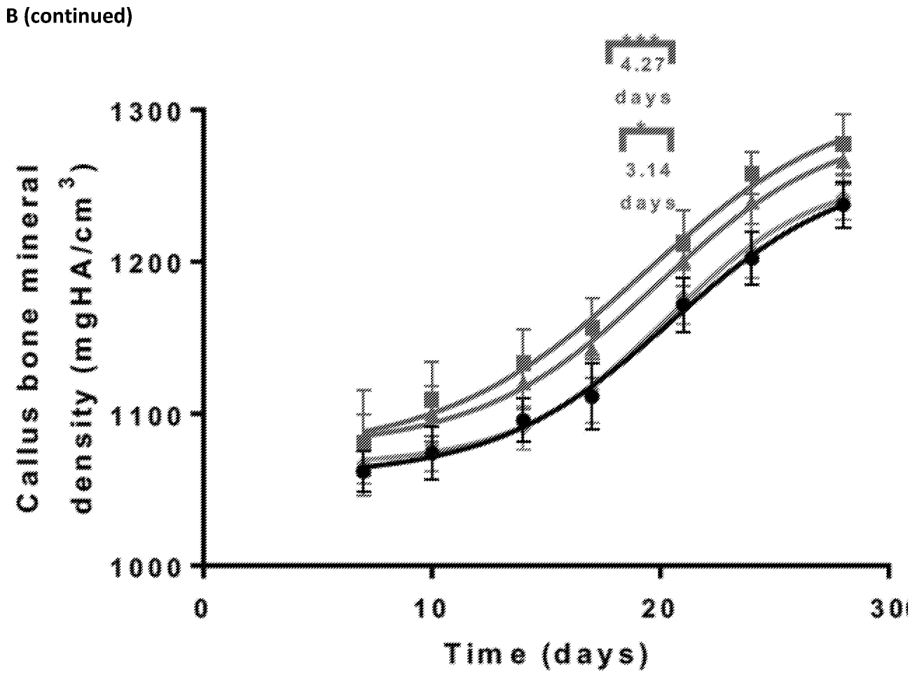

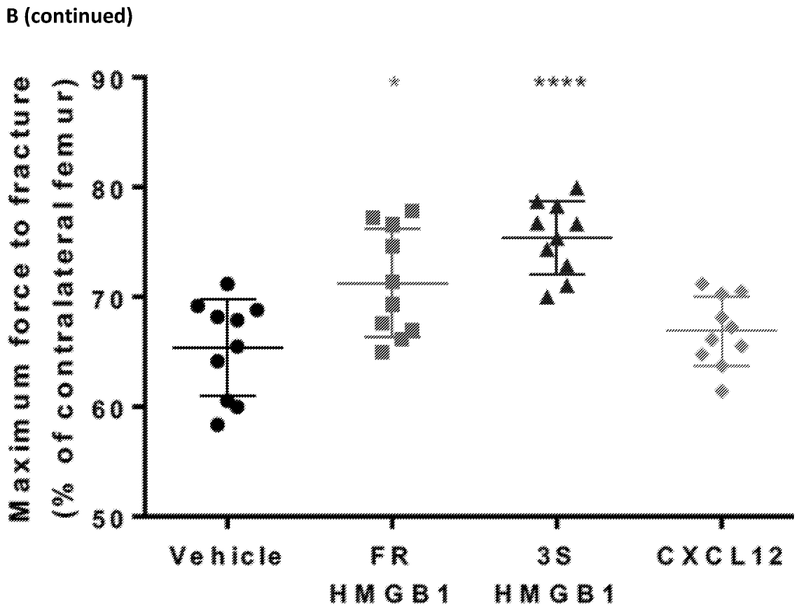

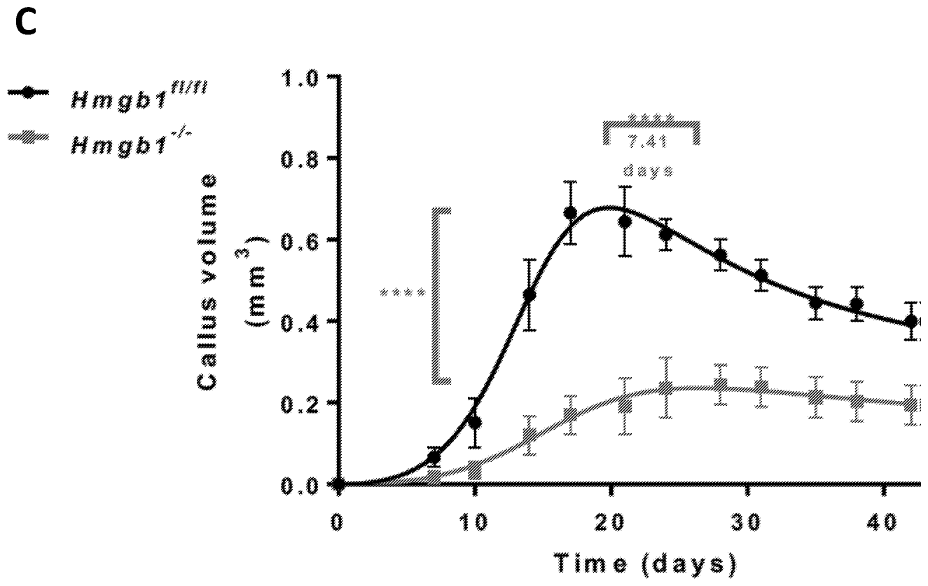

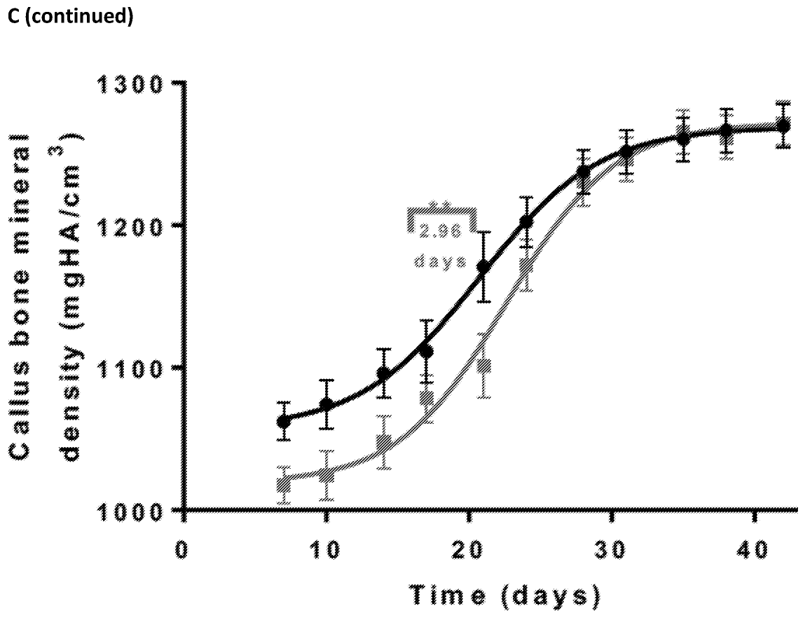

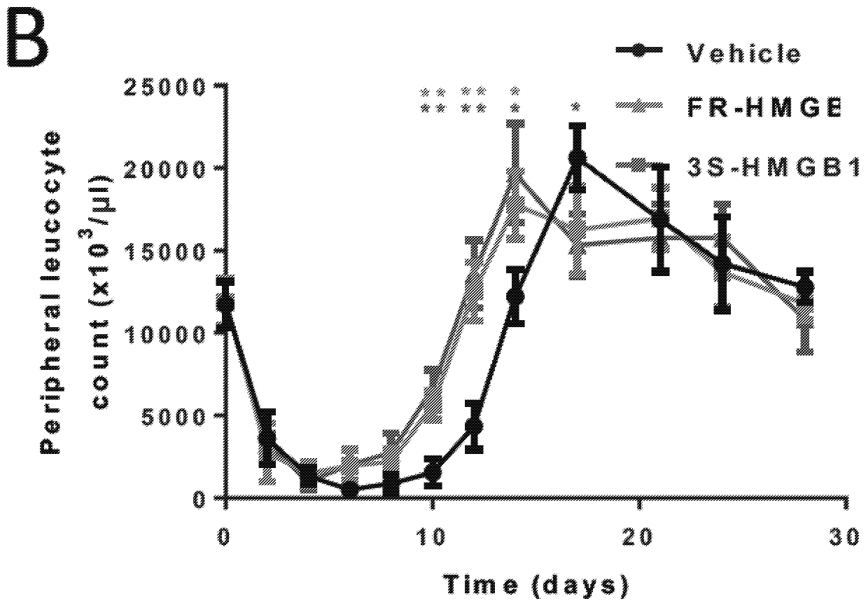

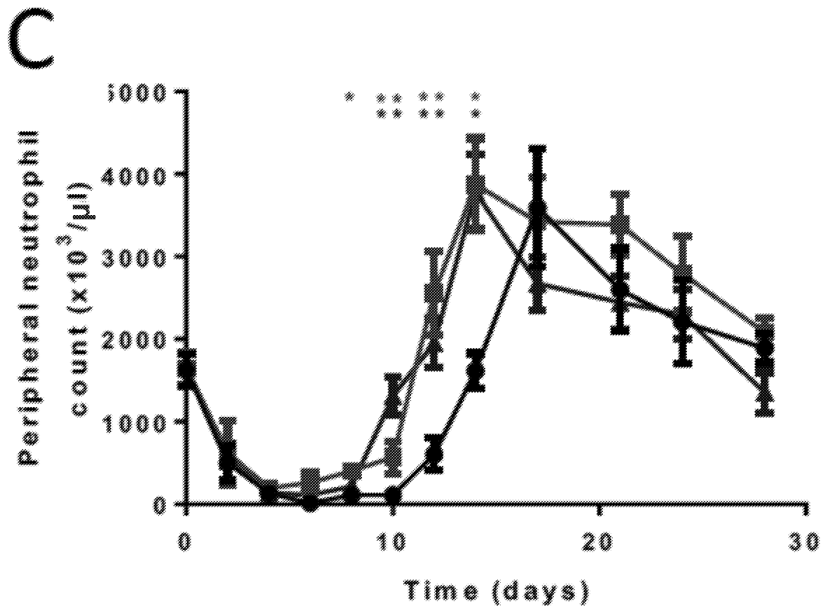

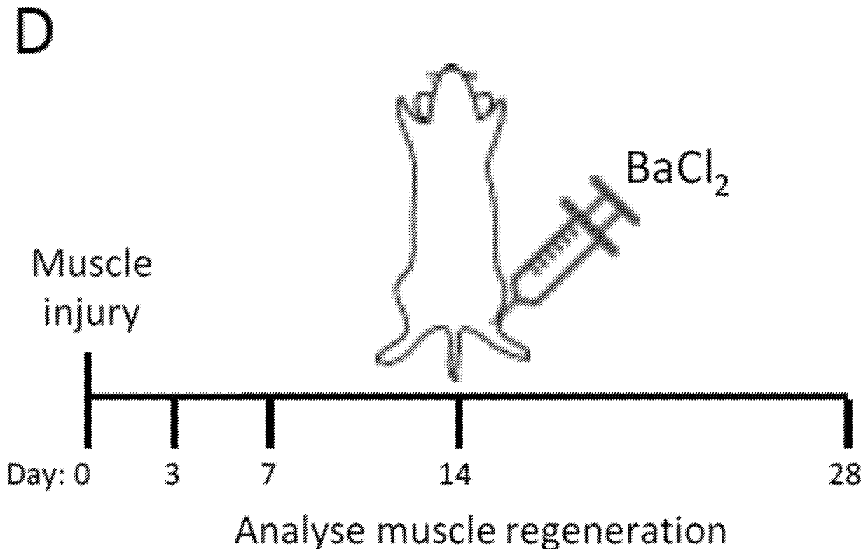

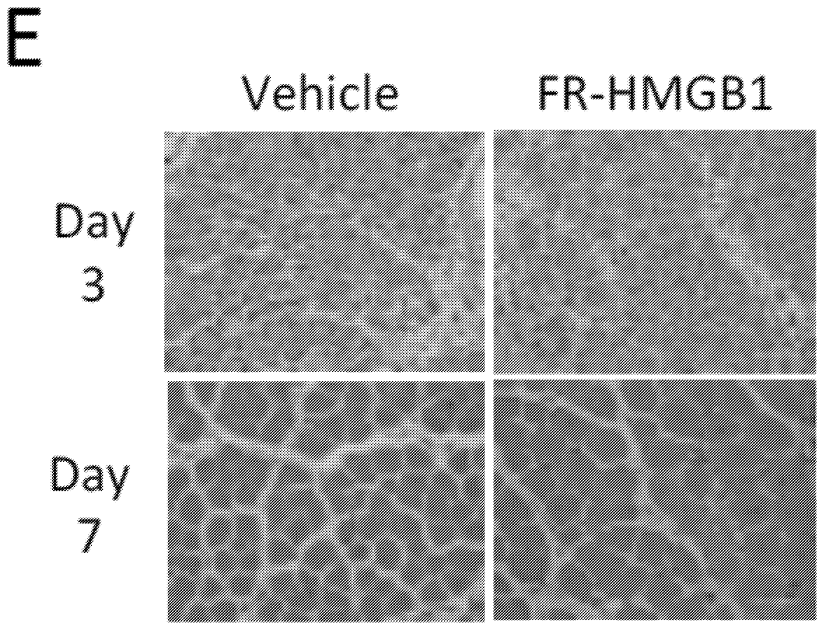

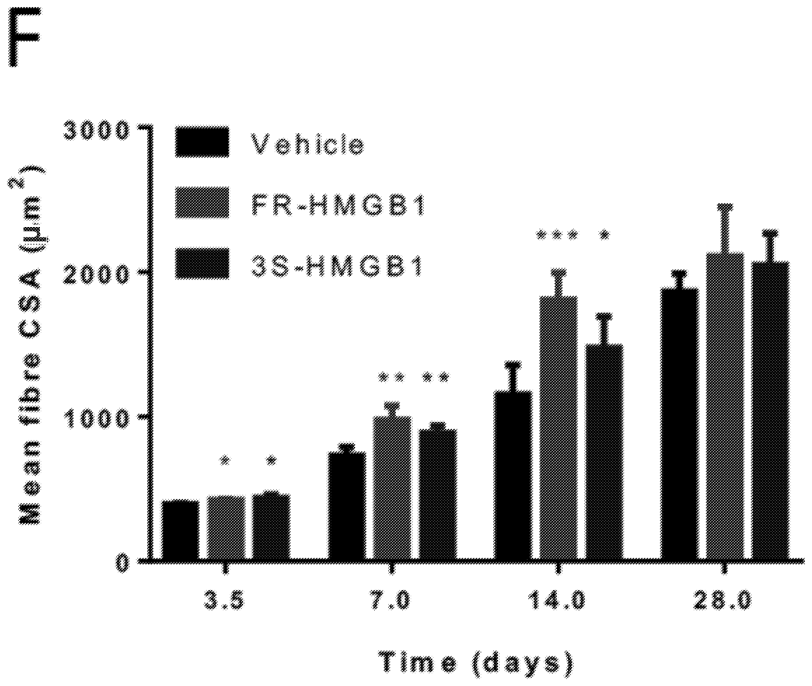

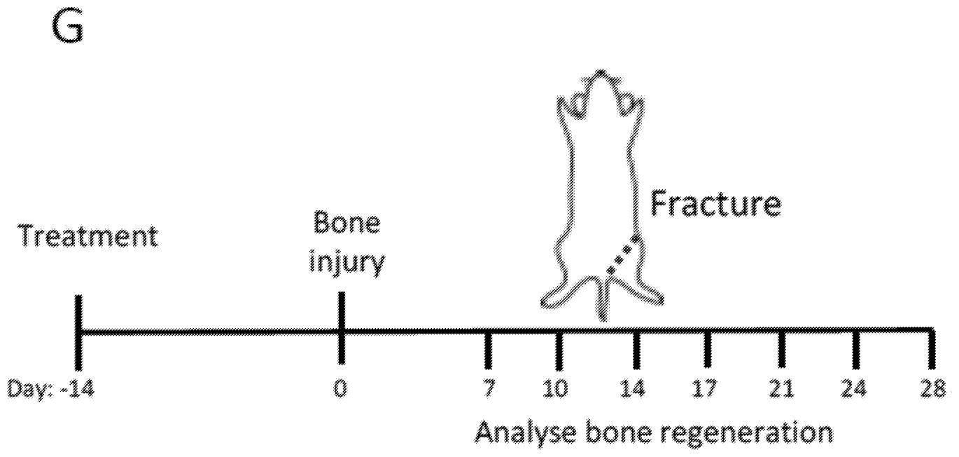

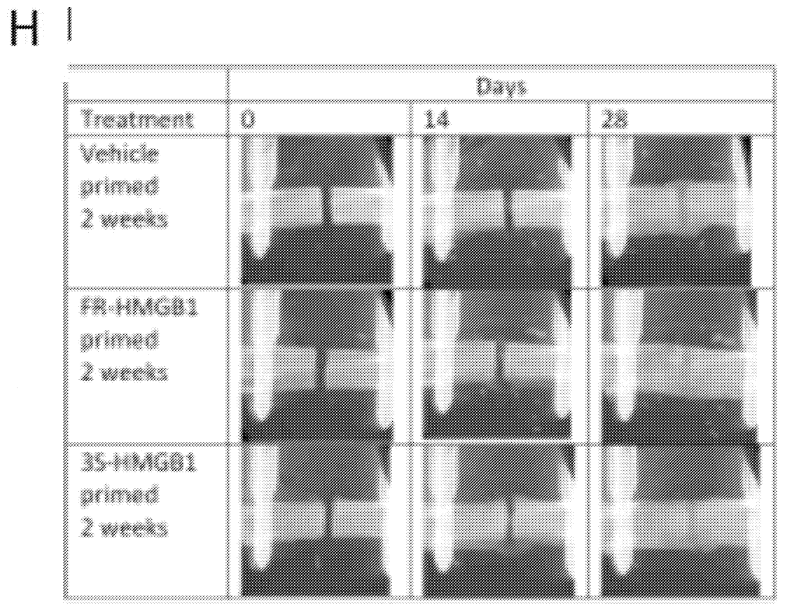

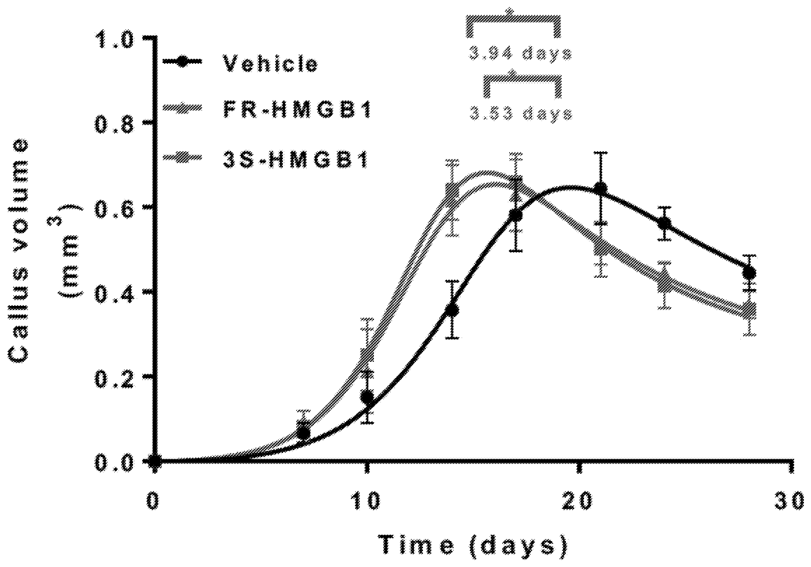

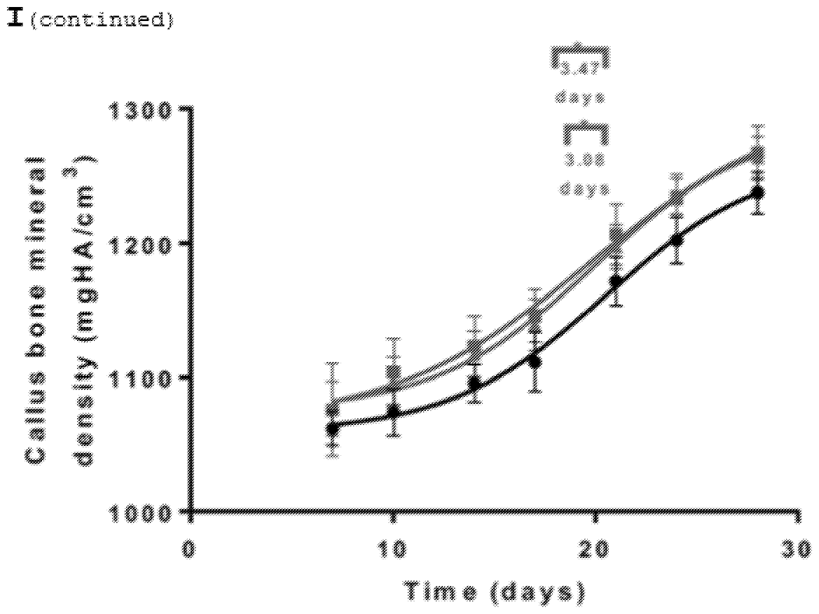

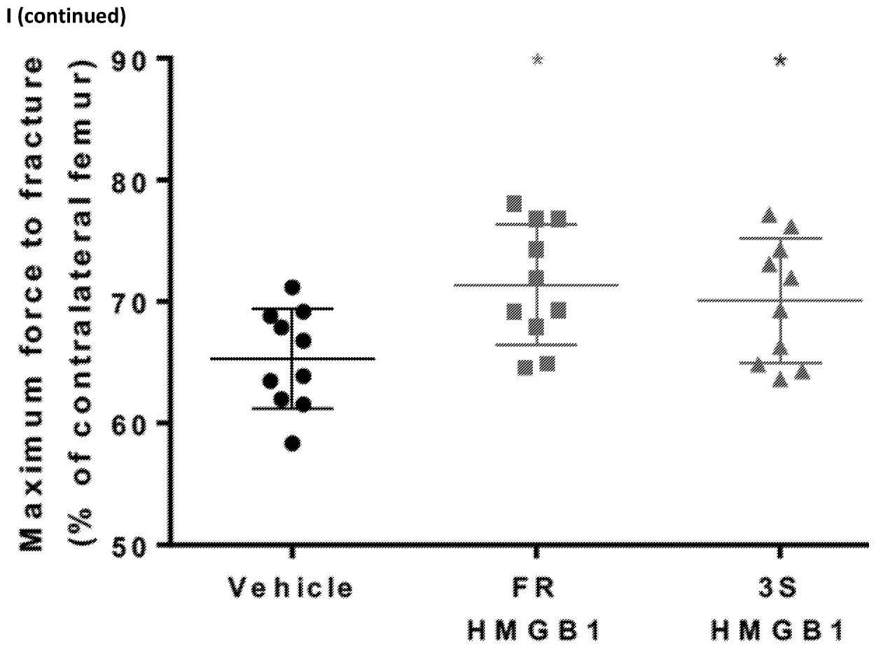

Fracture healing is a good model of tissue regeneration (Einhorn 2015) and based on studies of the early events in fracture healing (Glass 2011), including the key role of neutrophils (Chan 2015), we postulated that the alarmins HMGB1 and S100A8/A9 may play key roles in tissue regeneration. HMGB1 is a highly conserved ubiquitous and abundant non-histone nuclear architectural protein that forms part of the transcription machinery (Harris 2012). S100A8/A9 proteins are calcium binding proteins that make up 40% of neutrophil cytoplasmic content (Edgeworth 1991). Both these alarmins have been associated with regulating skeletal cells (Chan 2012, Zreiqat 2007). Elevated levels of HMGB1 and S100A8/A9 were found in the circulation following fracture both in human patients and mice (FIGS. 1 A and B, and FIGS. 5 A and B).

### HMGB1 Primes Human MSCs for Osteogenic Differentiation.

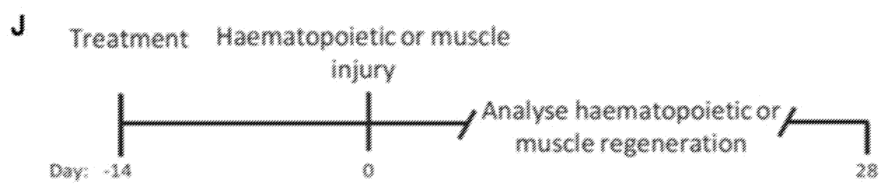

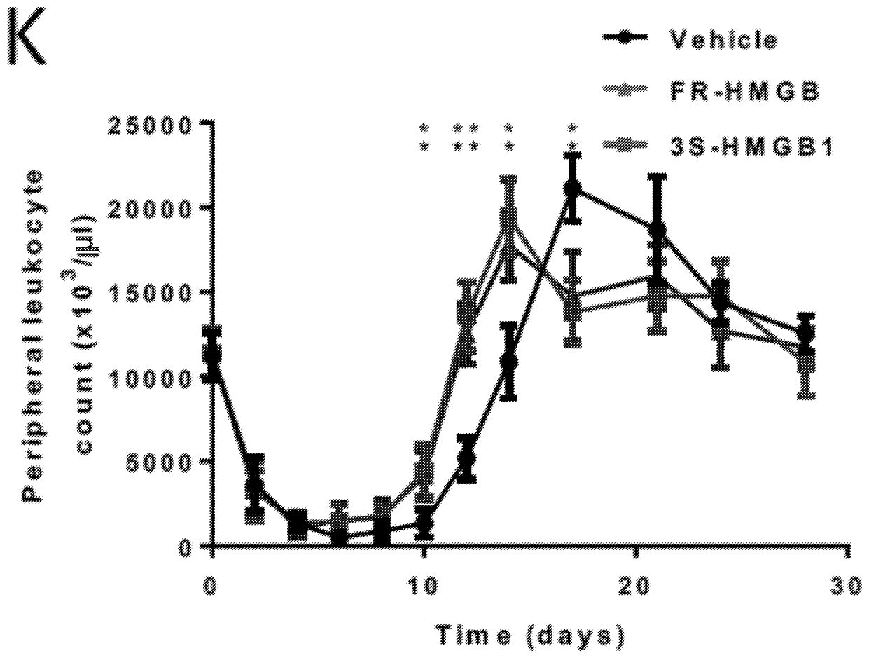

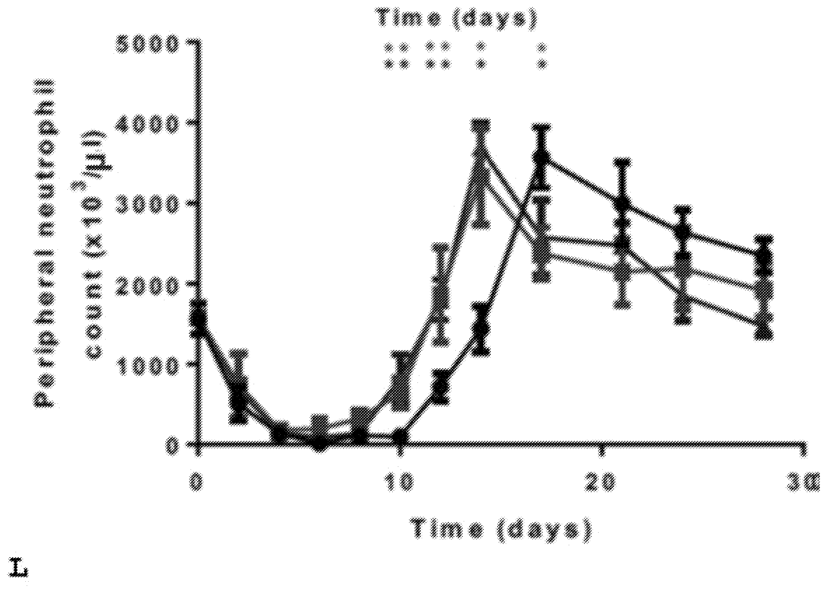

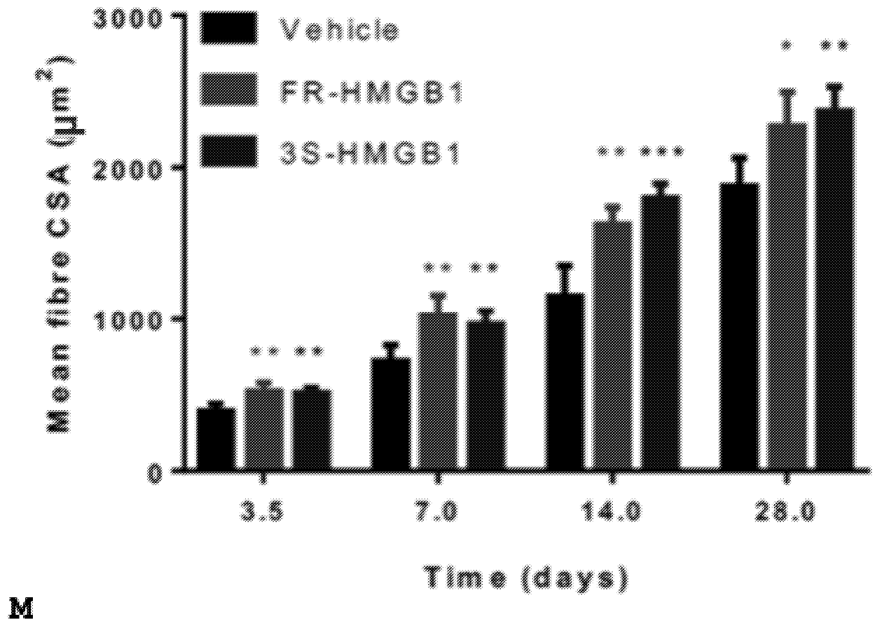

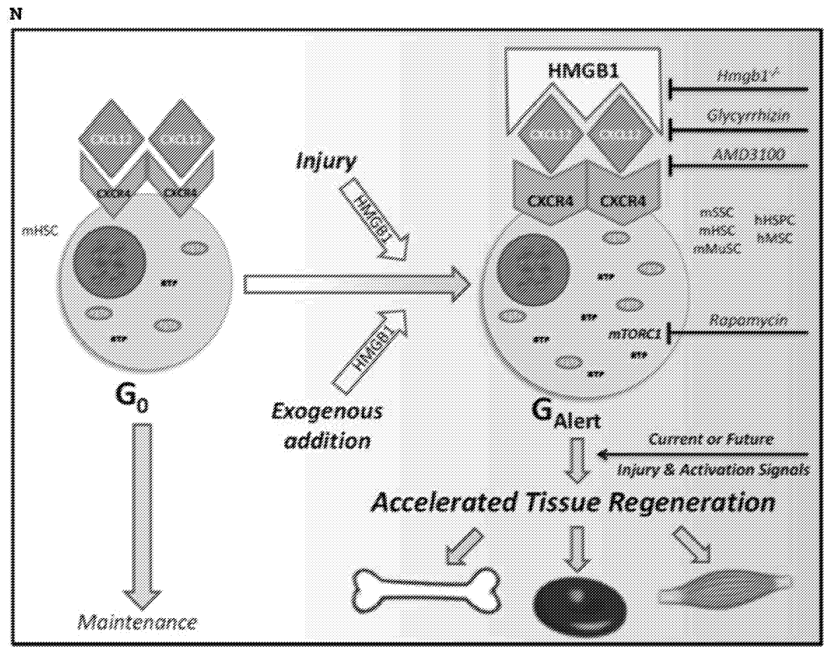

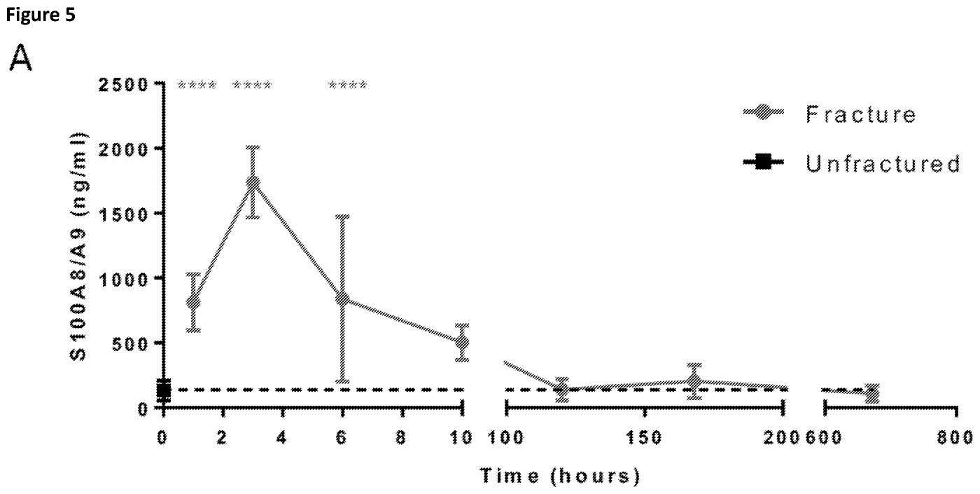

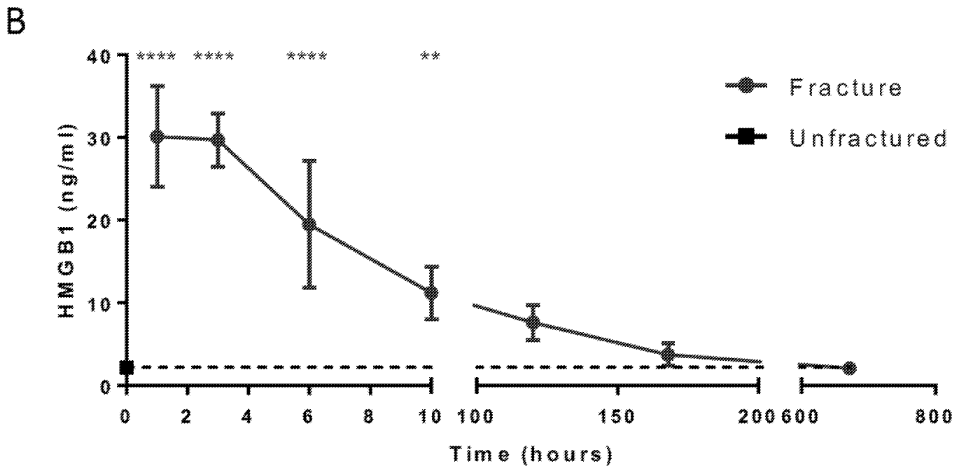

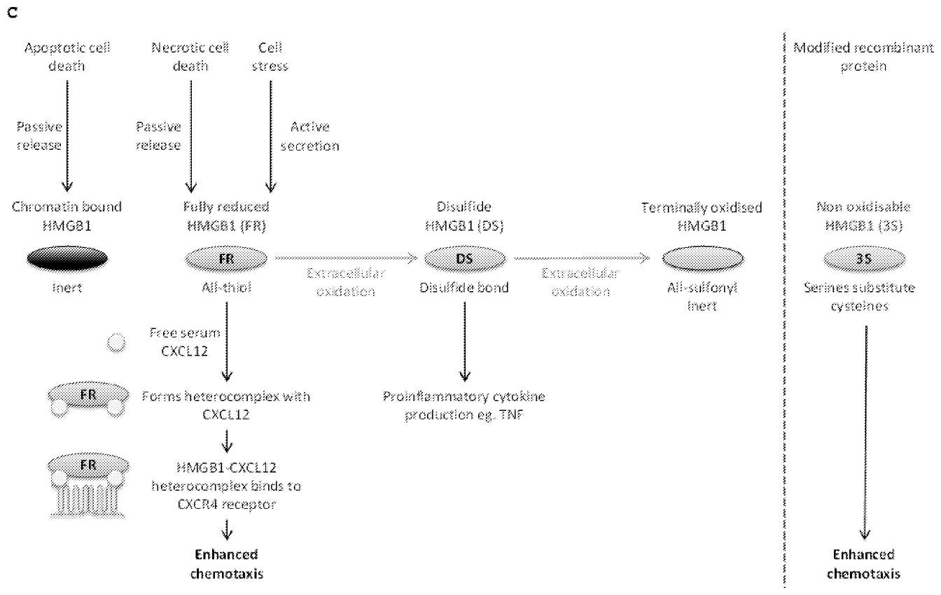

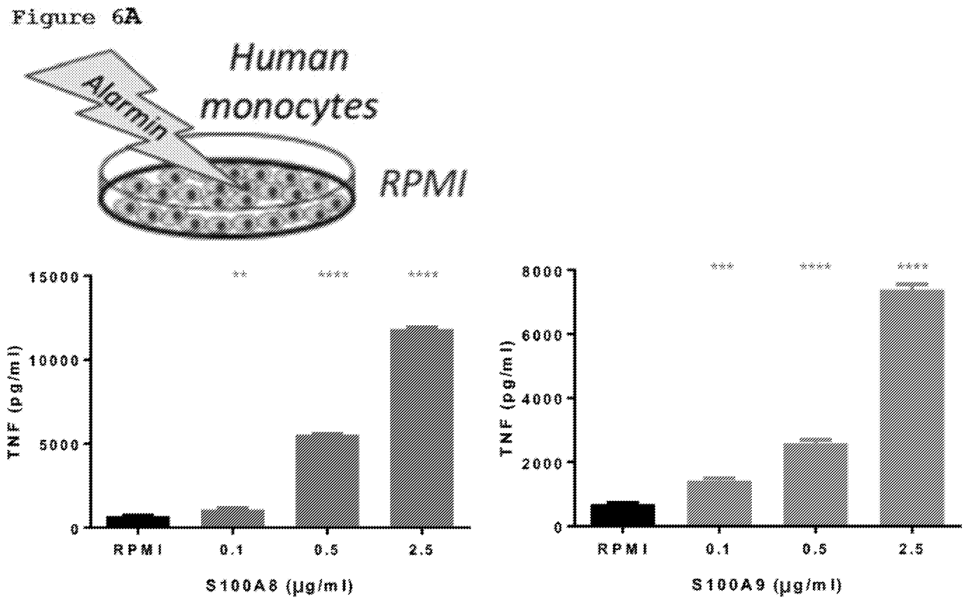

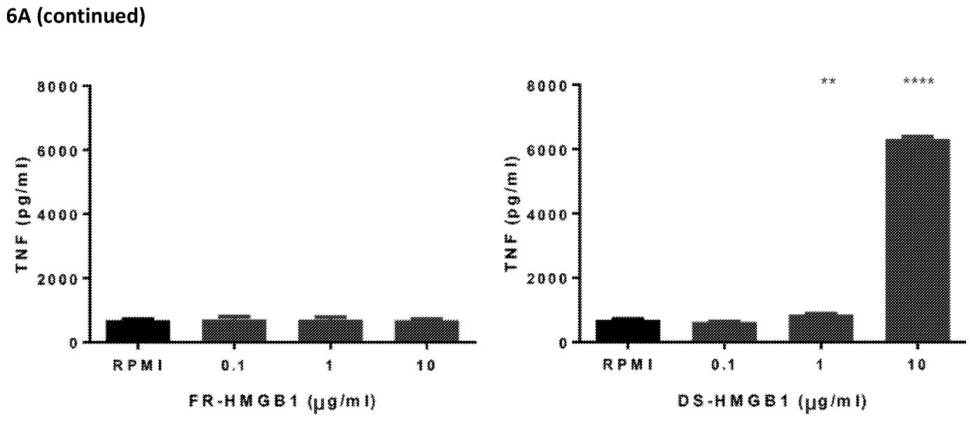

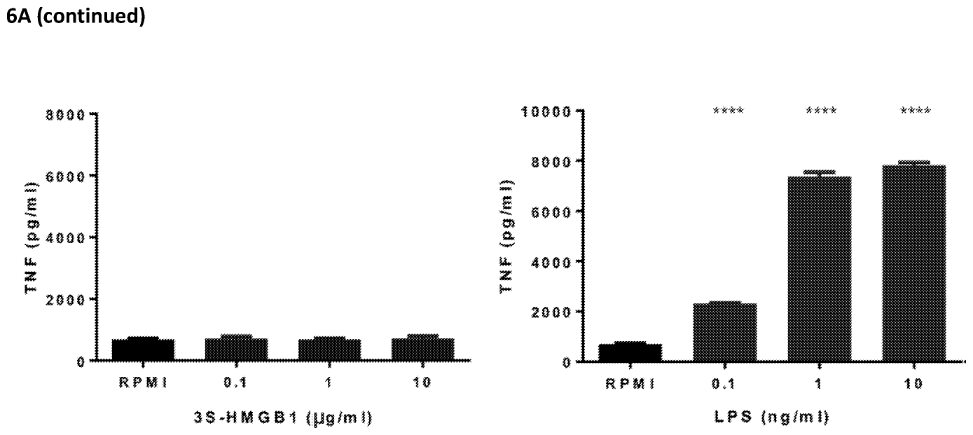

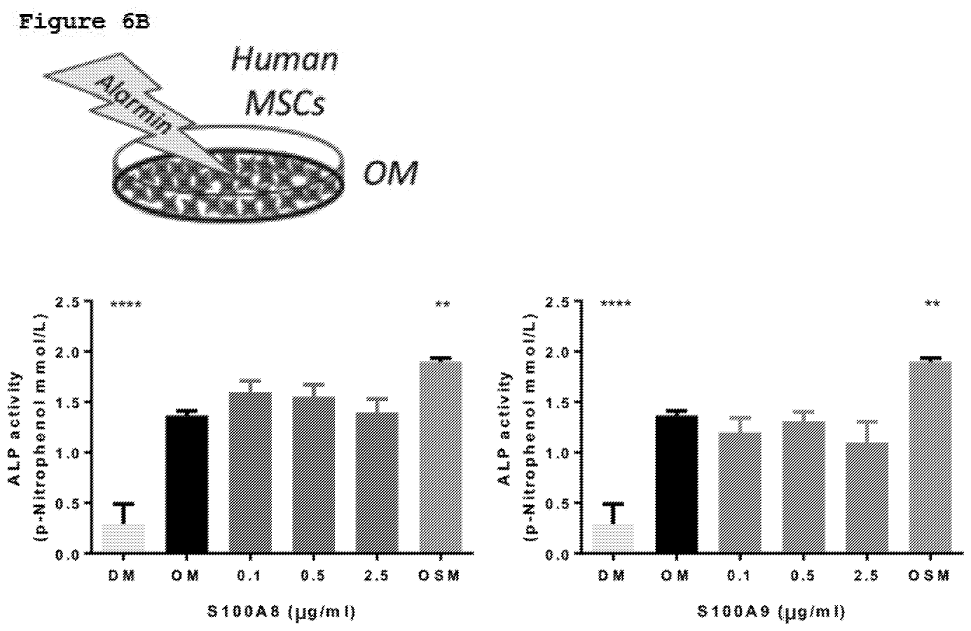

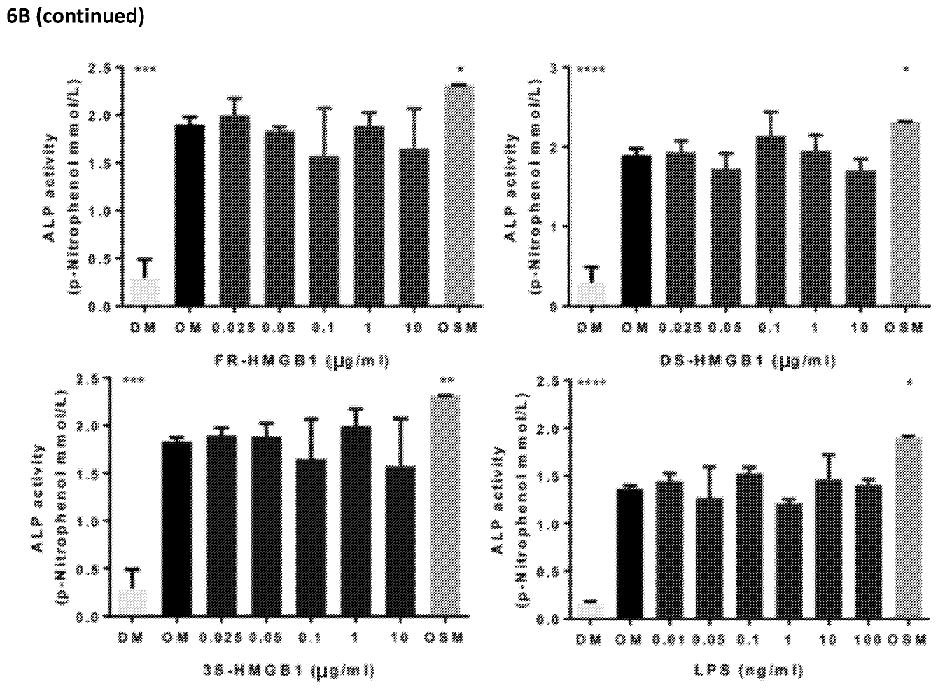

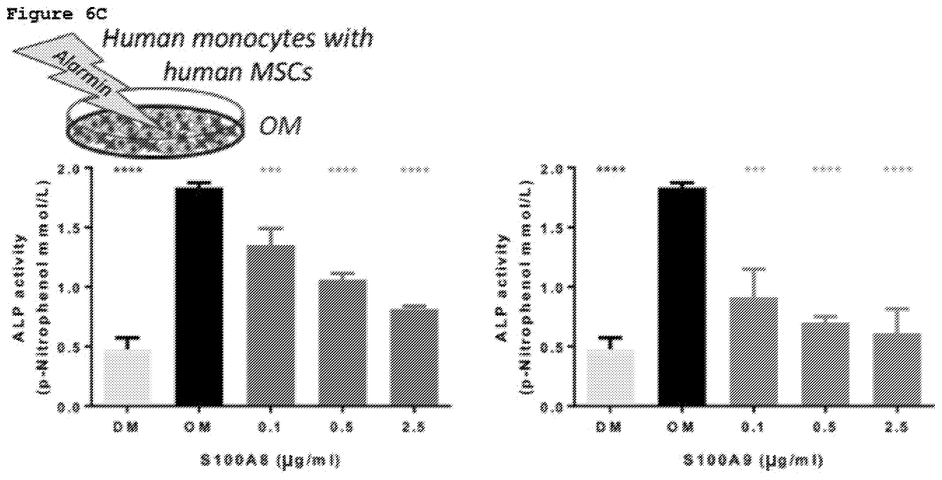

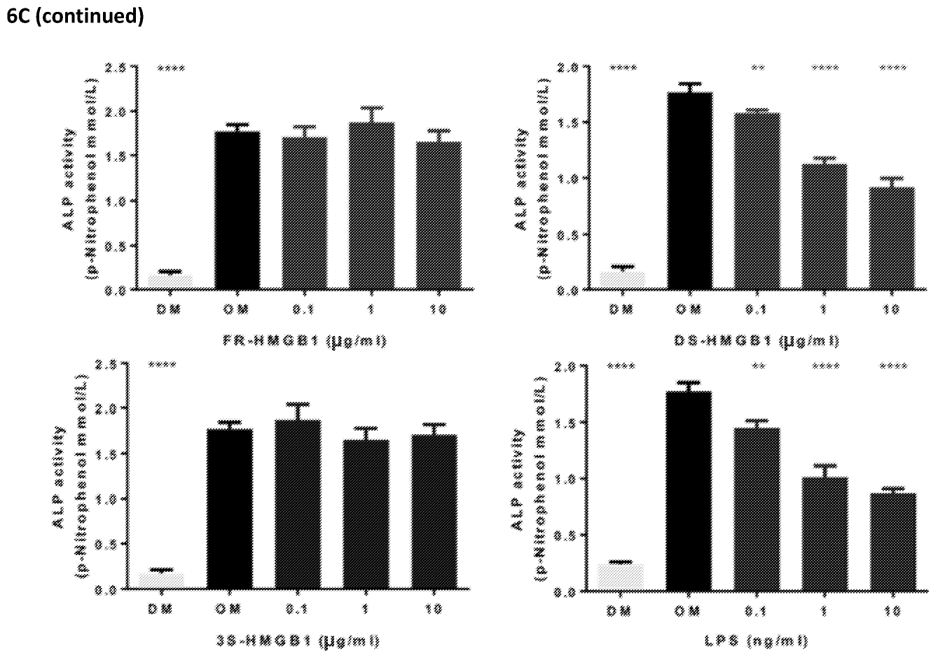

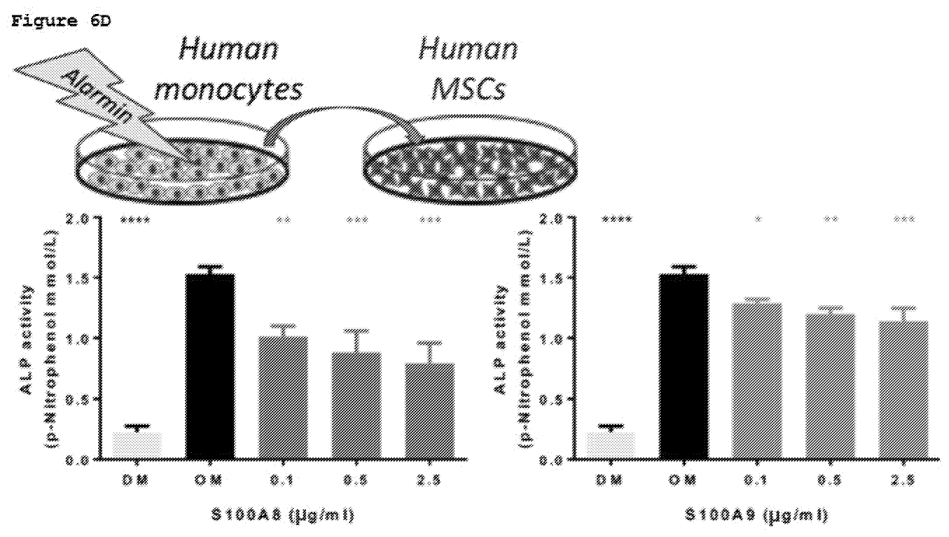

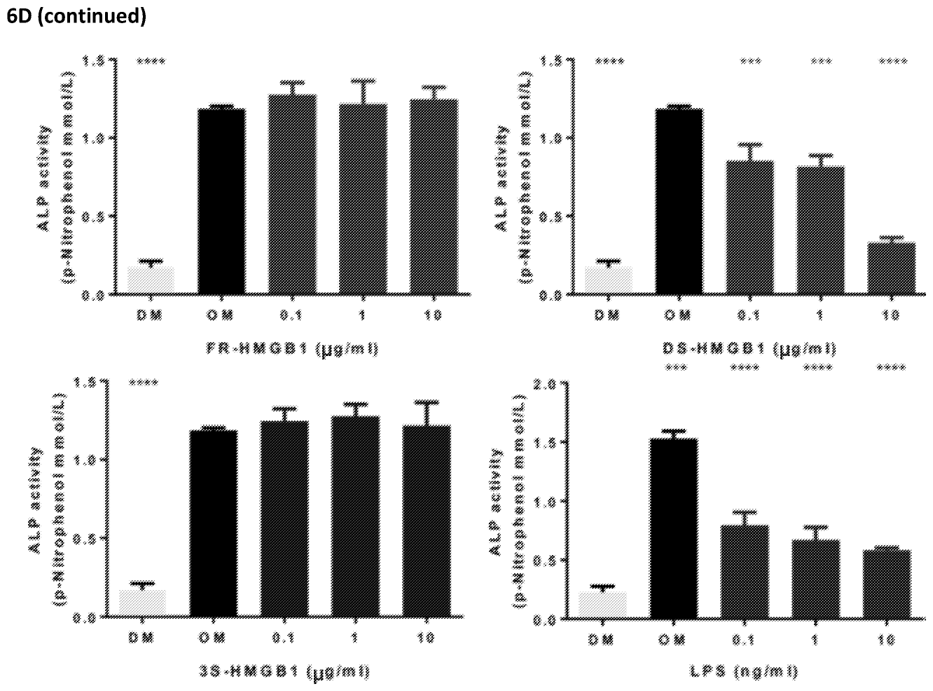

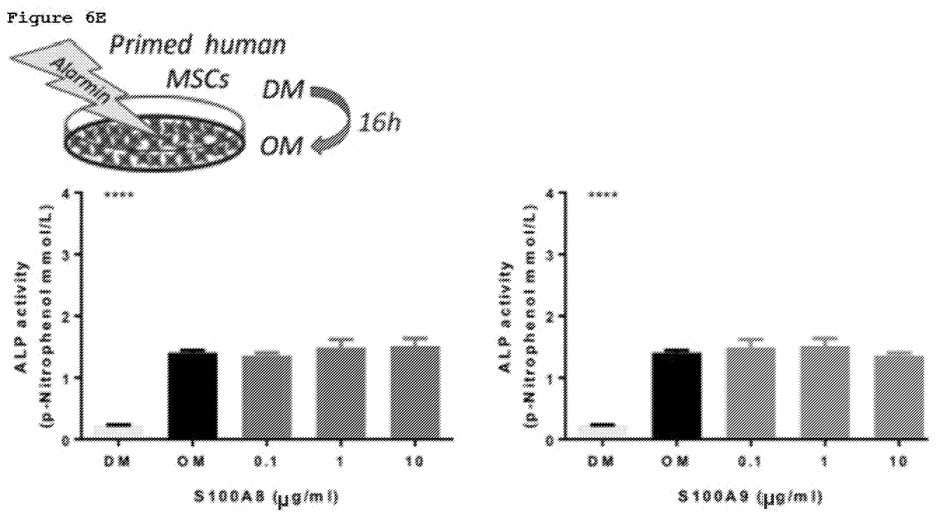

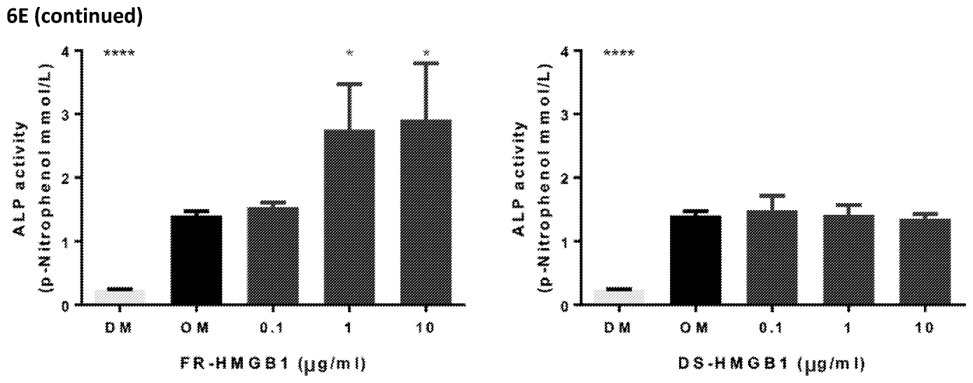

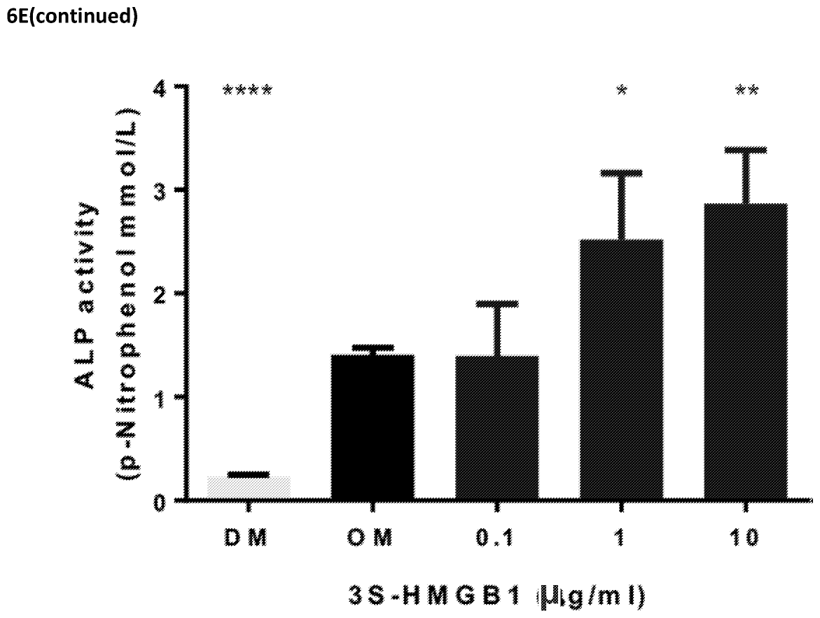

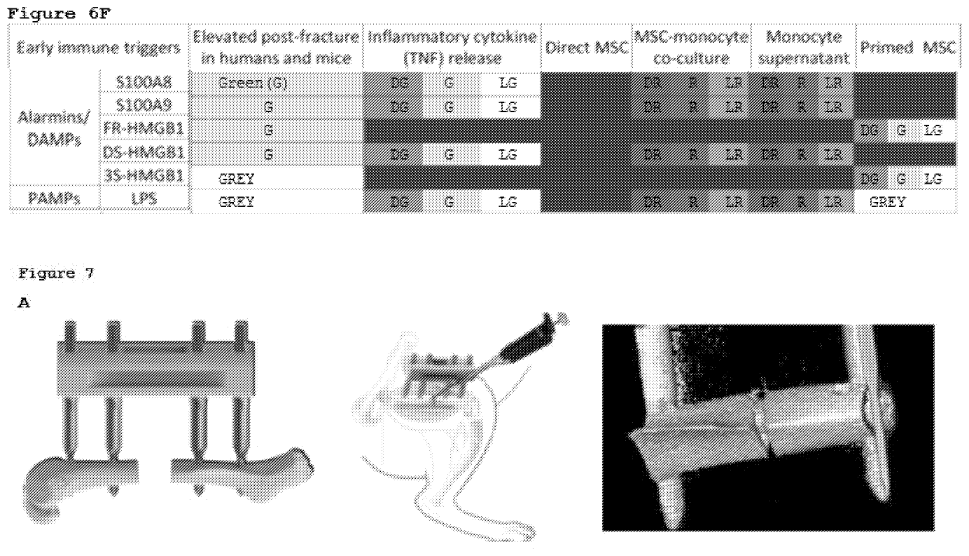

The regenerative potential of these alarmins were screened in humans by assessing the osteogenic differentiation of primary human mesenchymal stromal/stem cells (hMSCs) (FIG. 1C and FIG. 6). Different redox forms of HMGB1 were tested because they are known to have contrasting effects (Venereau 2012). Fully reduced (FR) all-thiol HMGB1 promotes chemotaxis (Venereau 2012), whereas partially oxidized HMGB1 with a disulfide bond (DS) induces proinflammatory cytokine production (FIG. 5C and FIG. 6A) (Venereau 2012). To confirm that the effect of FR-HMGB1 is due to its reduced state, we also used a recombinant non-oxidizable all-serine form (3S) of HMGB1 (Venereau 2012). Direct addition of alarmins to hMSCs did not promote osteogenic differentiation (FIG. 6B), whilst DS-HMGB1, S100AS, and S100A9 all inhibited this process in the presence of monocytes (FIG. 6C), as did the supernatants from alarmin-treated monocytes (FIG. 6D). Since alarmins are released before resident stem cells are exposed to most osteogenic signals in vivo, this temporal sequence was modeled in vitro and it was found that pre-exposure to only FR-HMGB1 or 3S-HMGB1, but not the proinflammatory DS-HMGB1, promoted osteogenic differentiation (FIGS. 1 E and F, and FIG. 6E). In vivo administration of FR-HMGB1 or 3S-HMGB1 was not found to lead to production of proinflammatory cytokines, in contrast to DS-HMGB1 (FIG. 7 I-K). These data suggest that only FR-HMGB1 or 3S-HMGB1, which cannot be oxidized and hence does not induce proinflammatory cytokine production in vitro (FIG. 5C and FIG. 6A) or in vivo (FIG. 7 I-K), are viable candidates to promote fracture healing if administered prior to the presence of potent osteogenic mediators.

Exogenous HMGB1 Accelerates Fracture Healing while Genetic Deletion of HMGB1 Delays Fracture Healing.

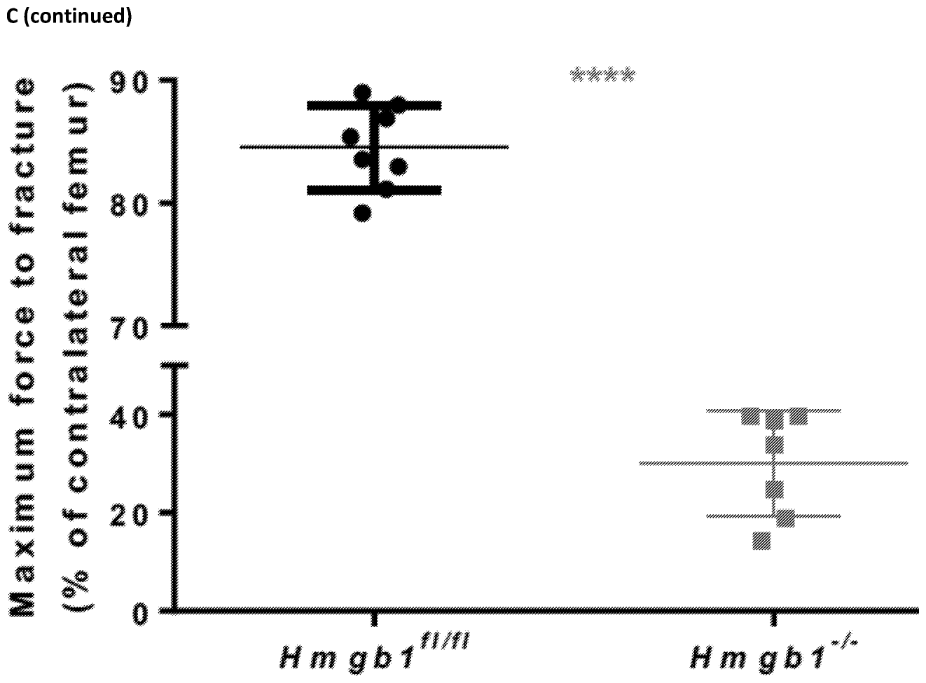

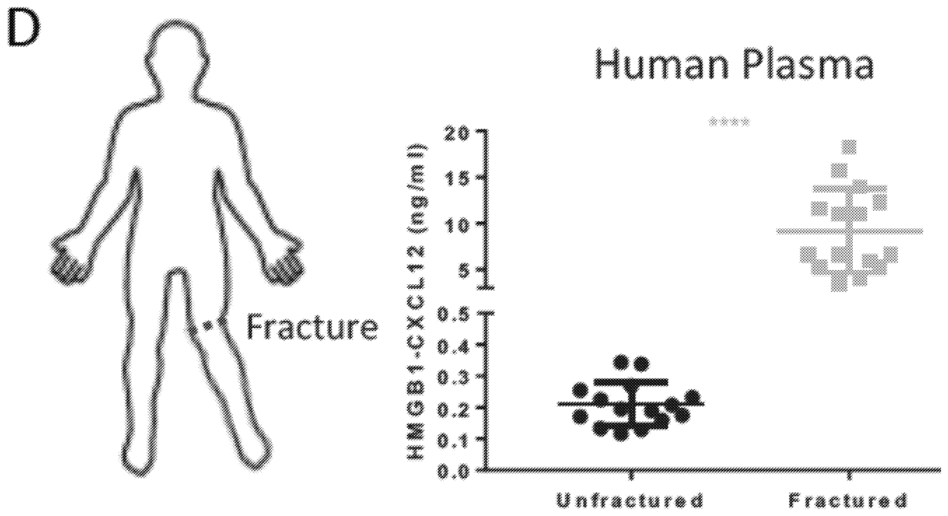

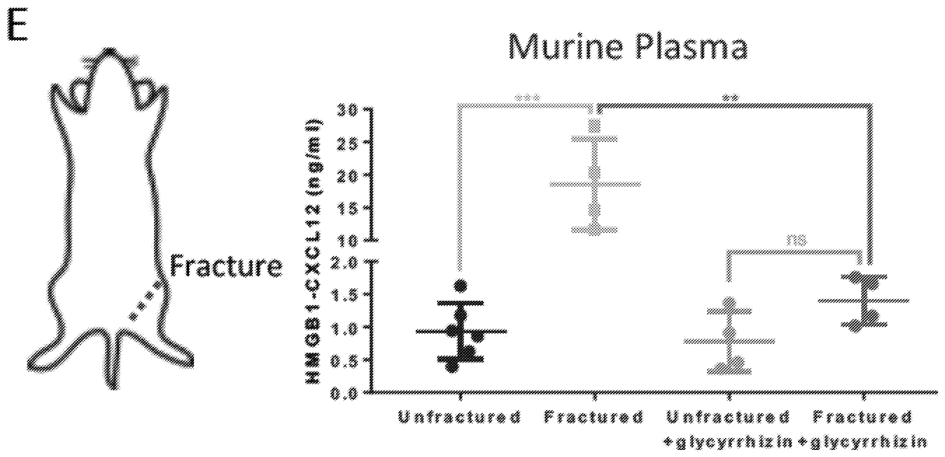

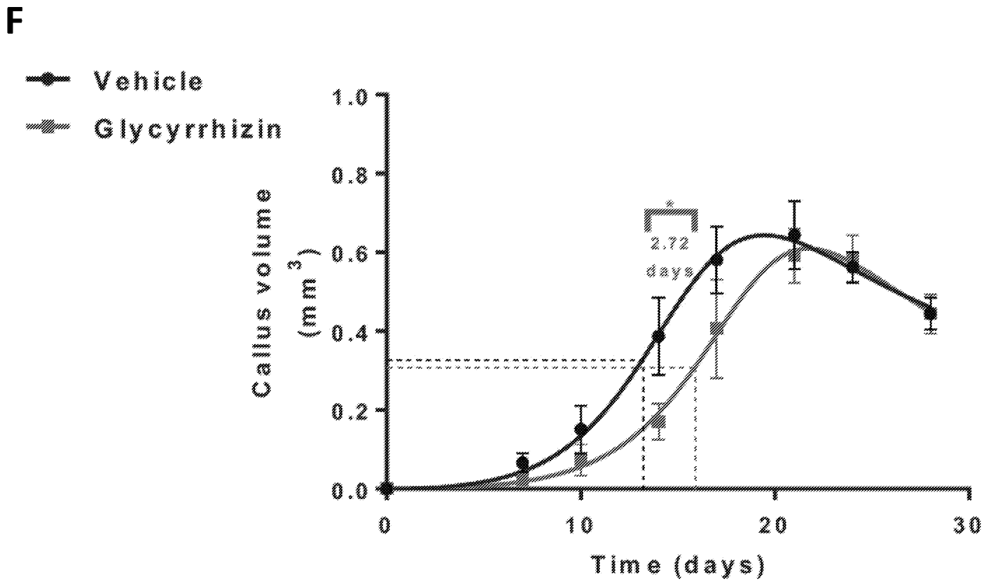

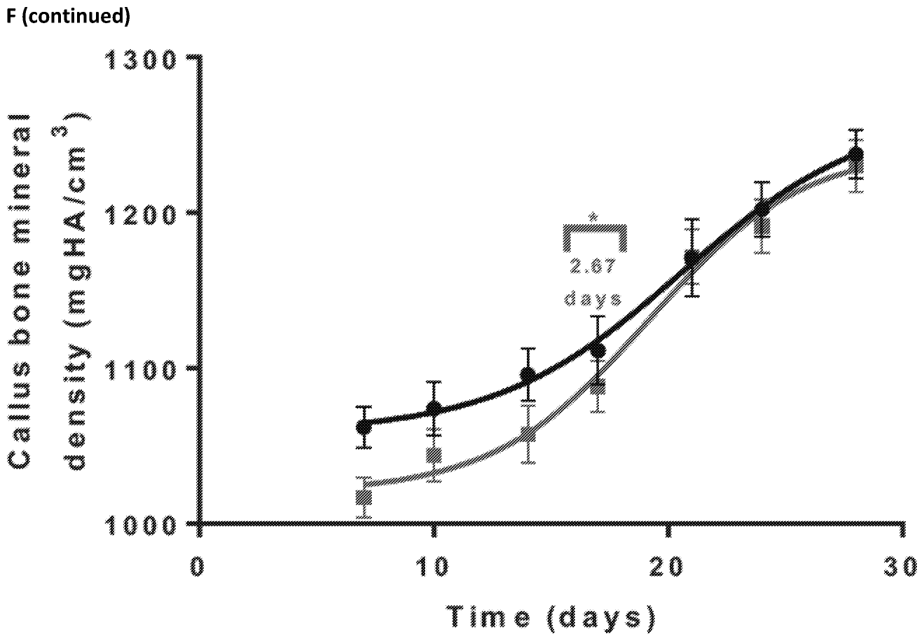

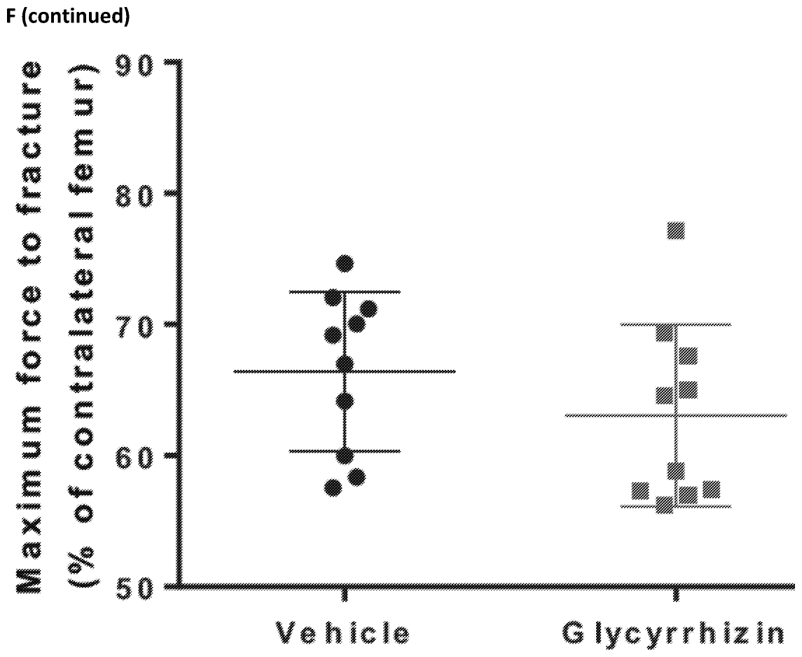

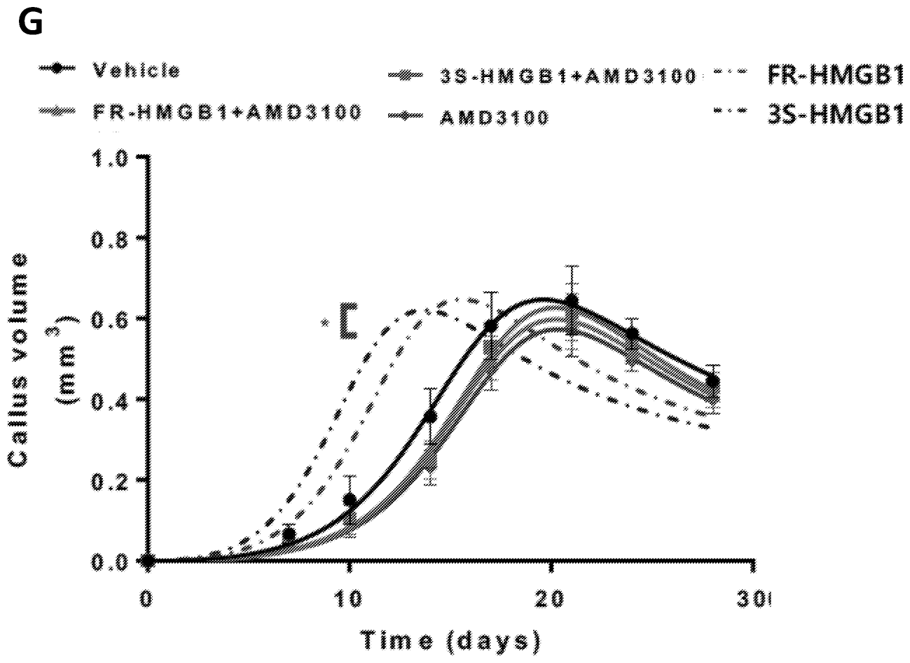

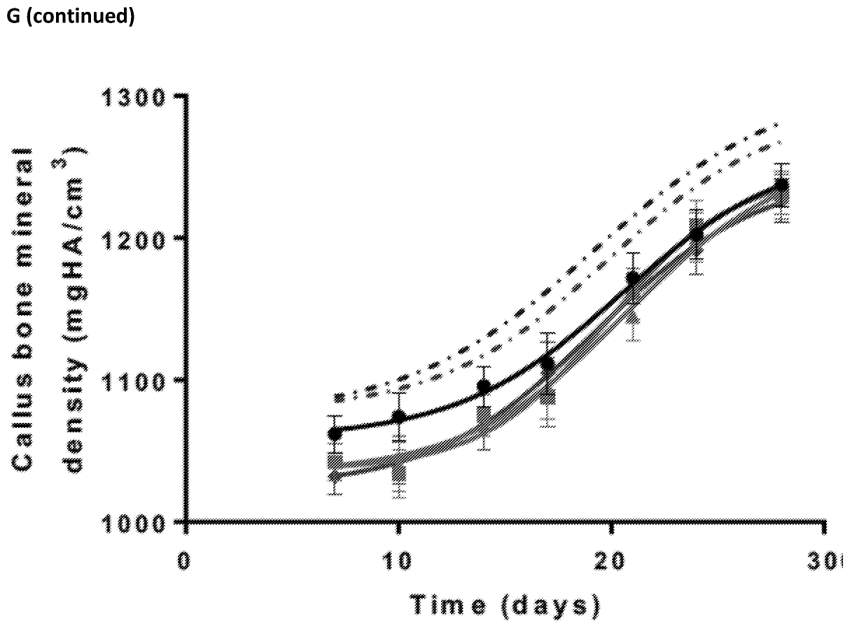

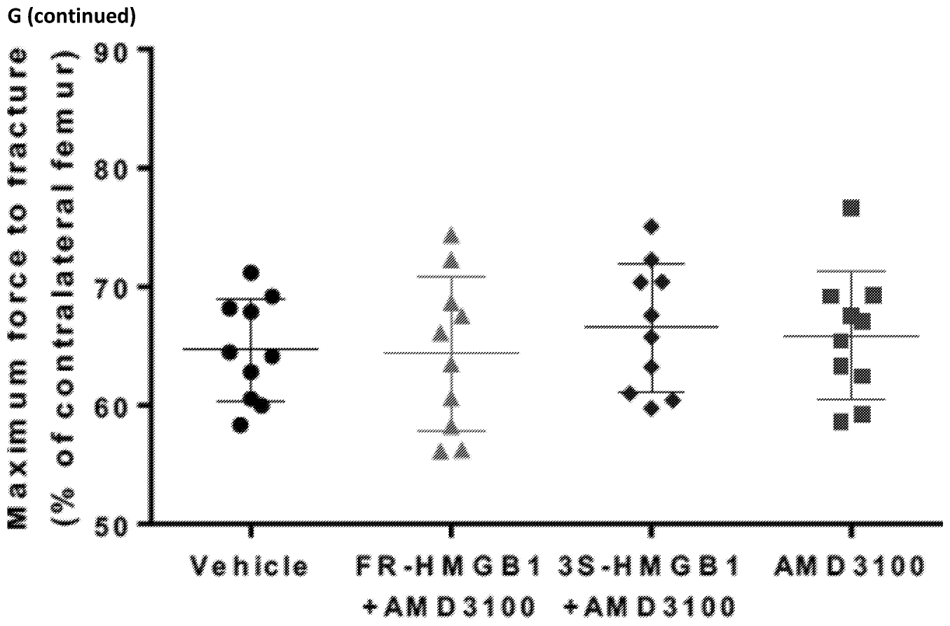

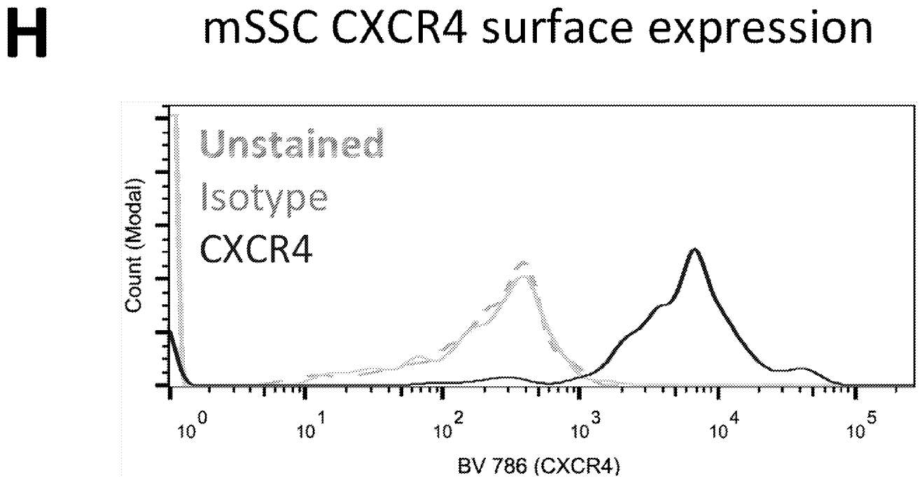

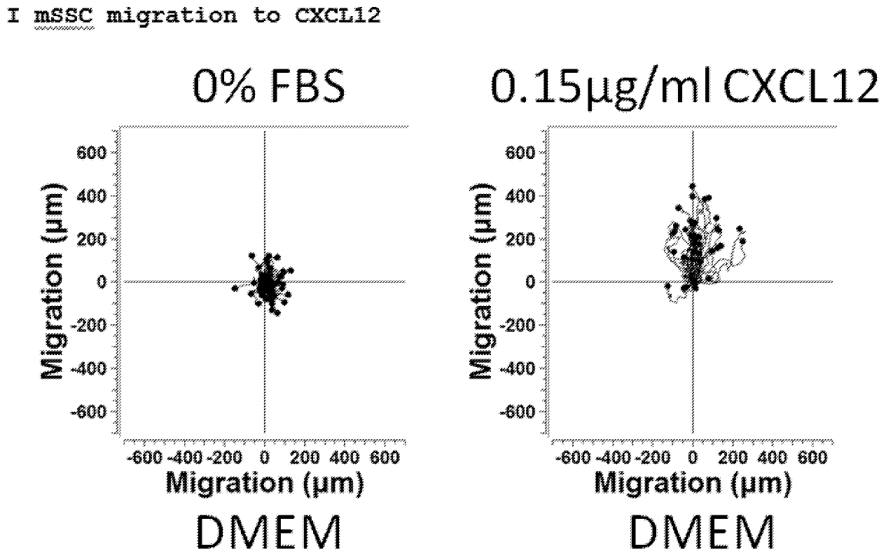

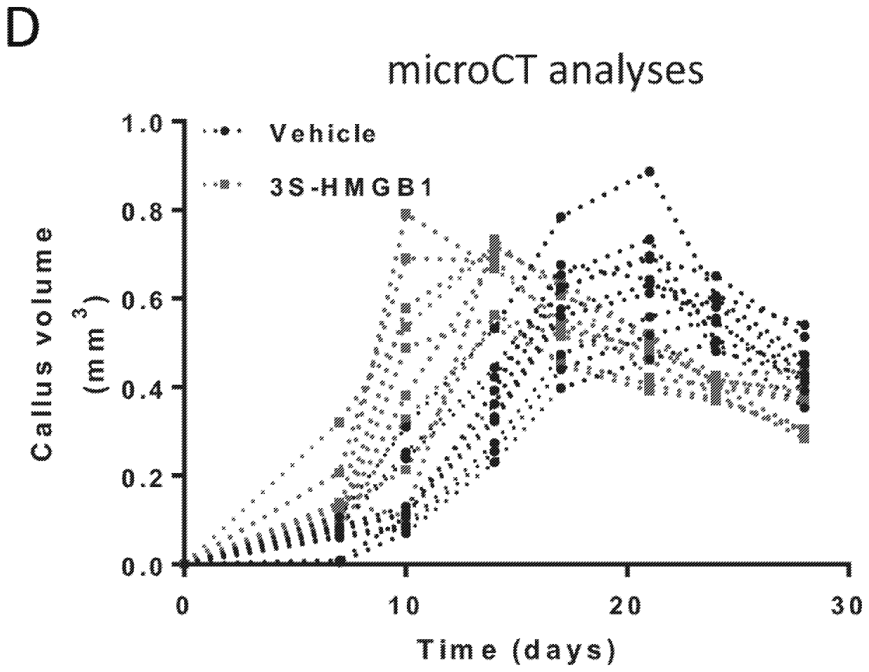

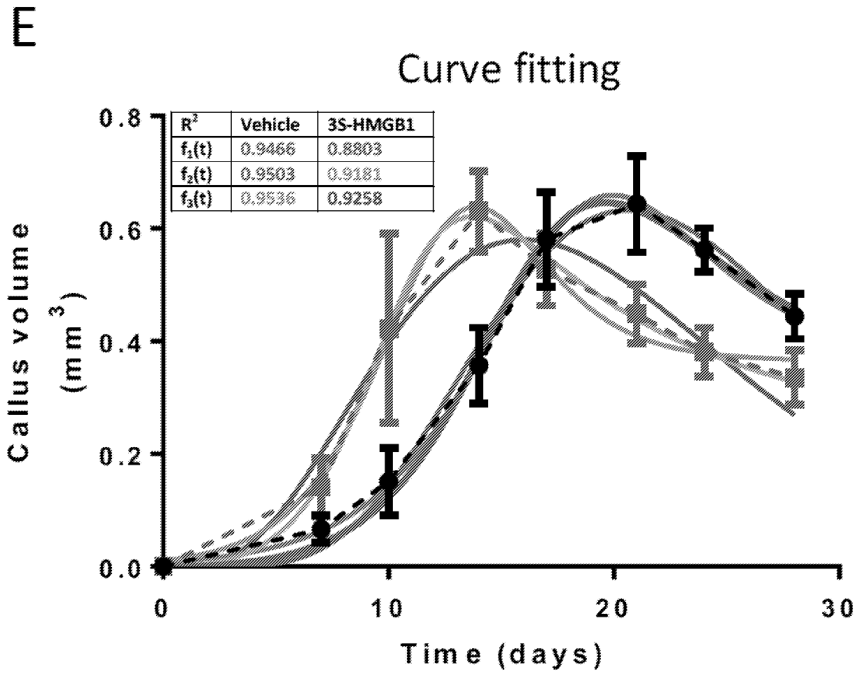

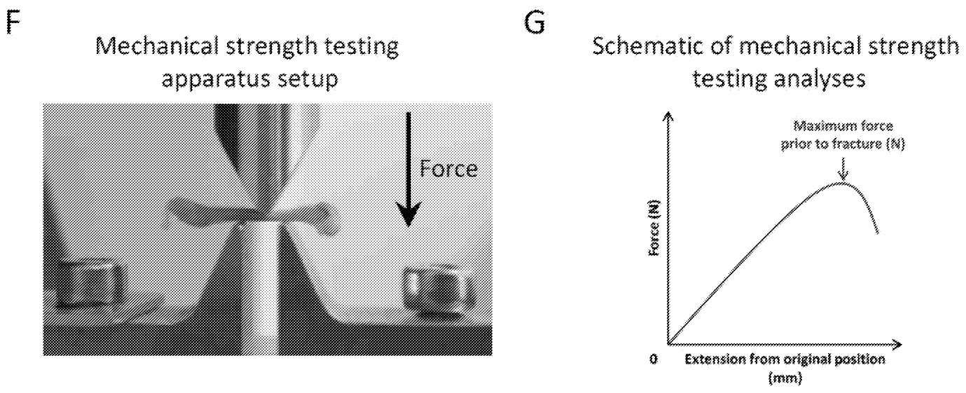

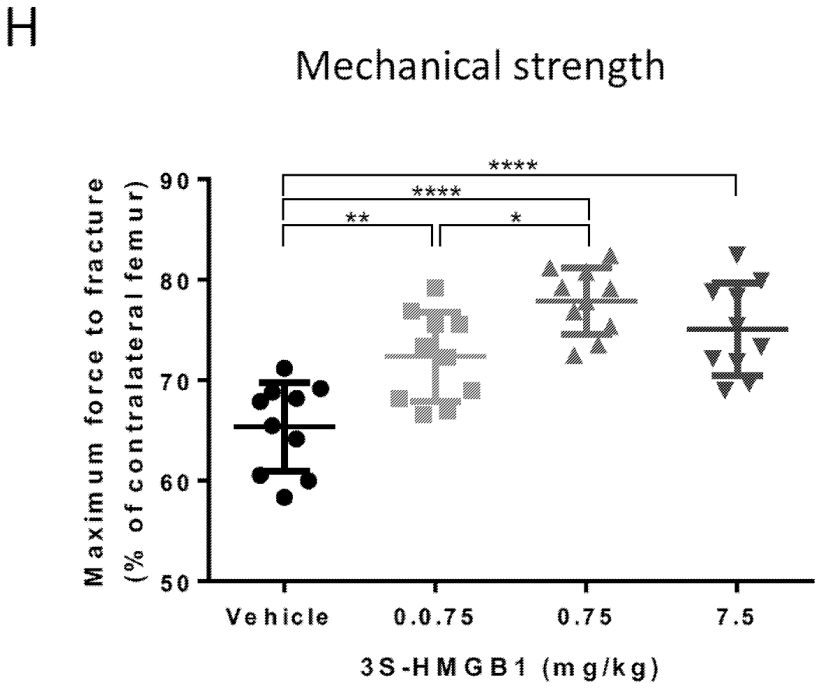

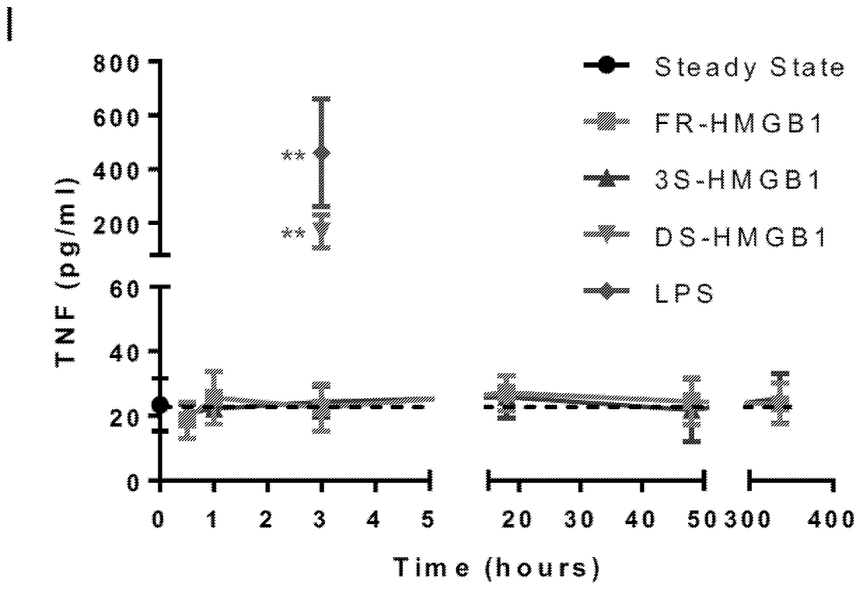

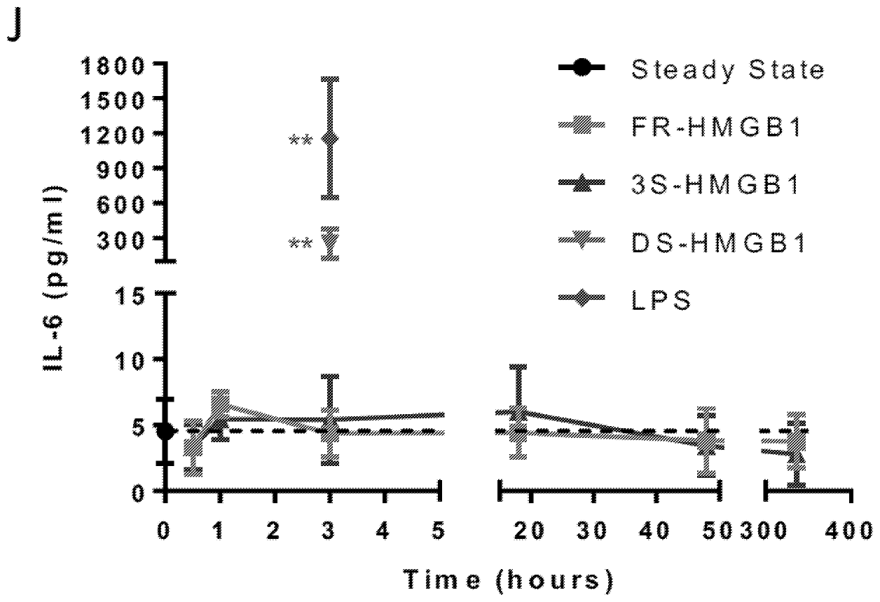

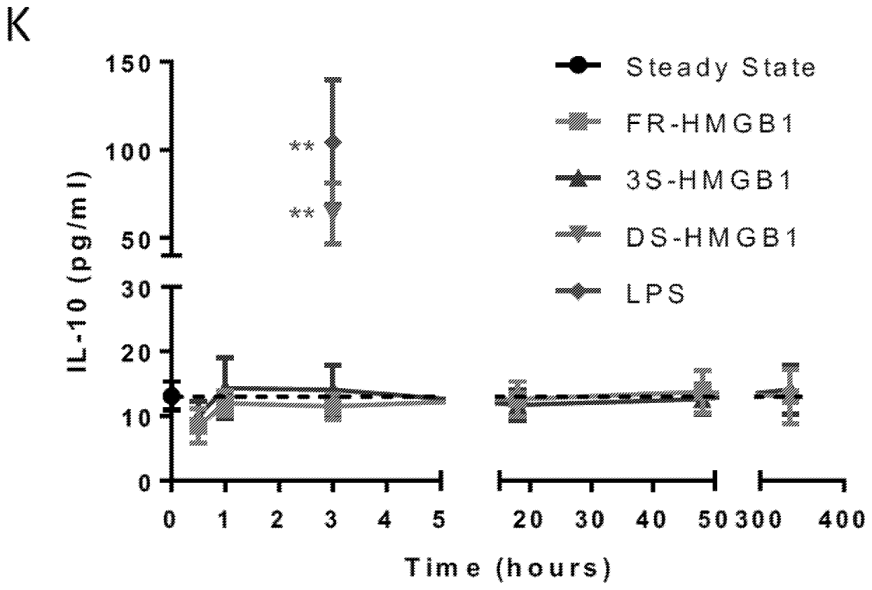

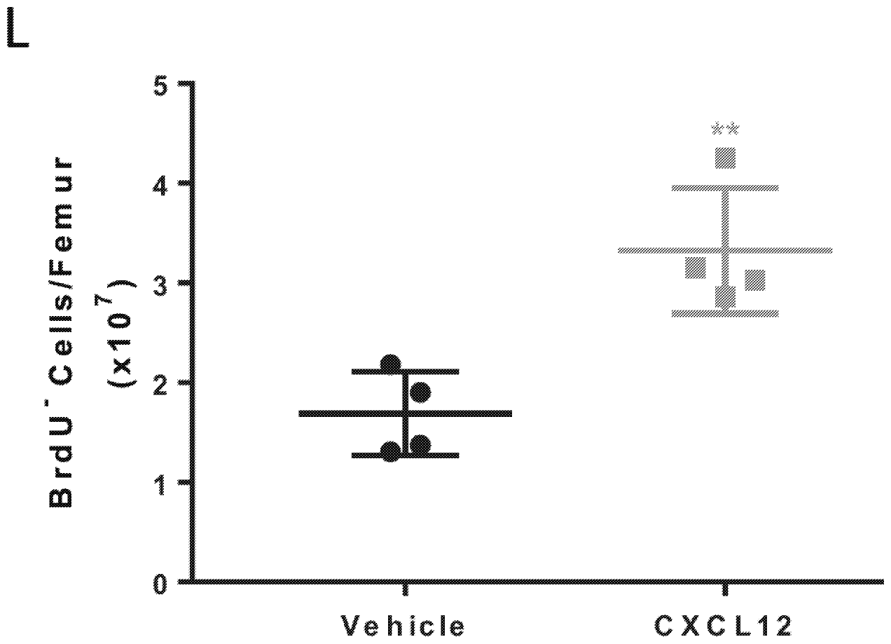

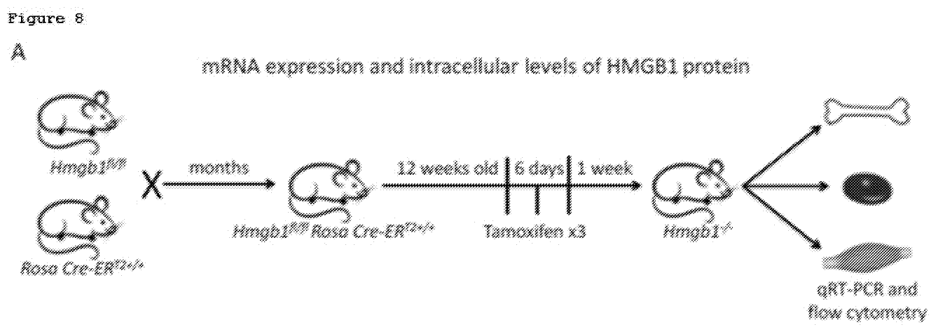

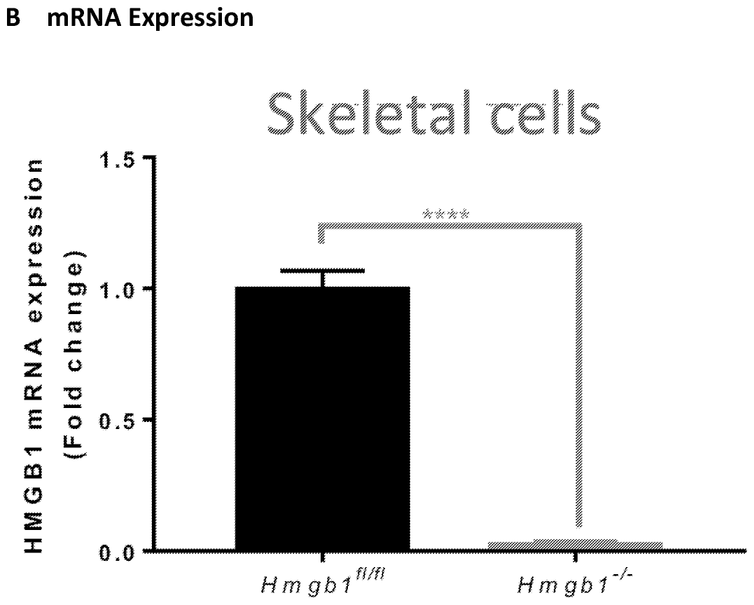

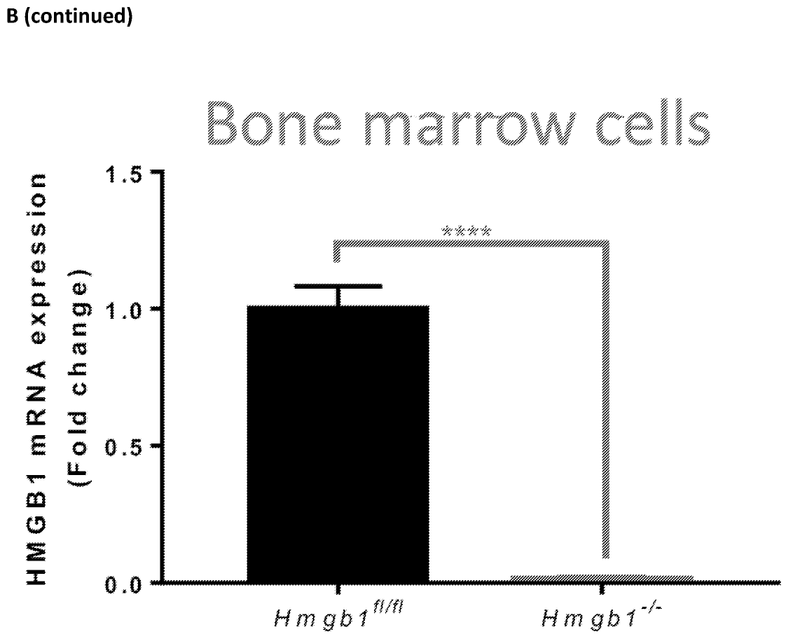

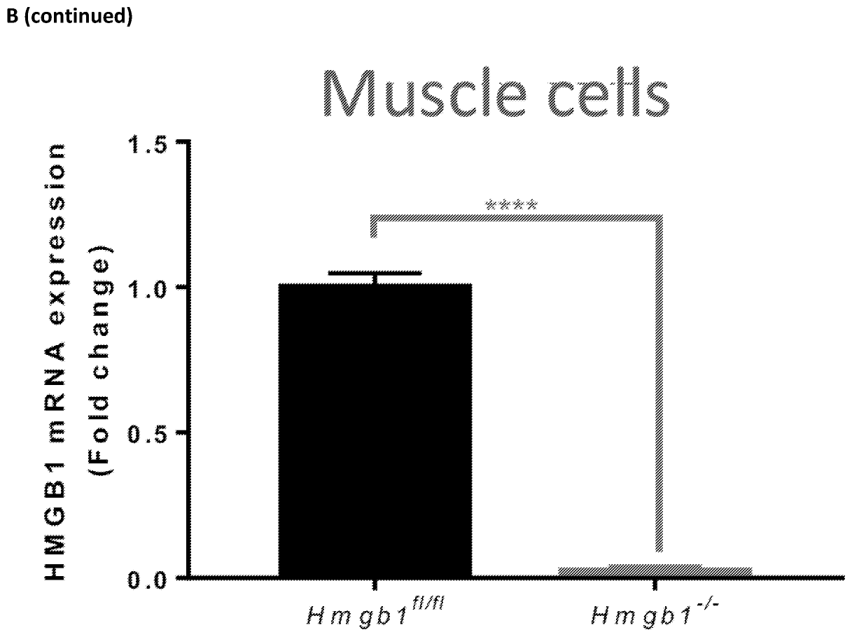

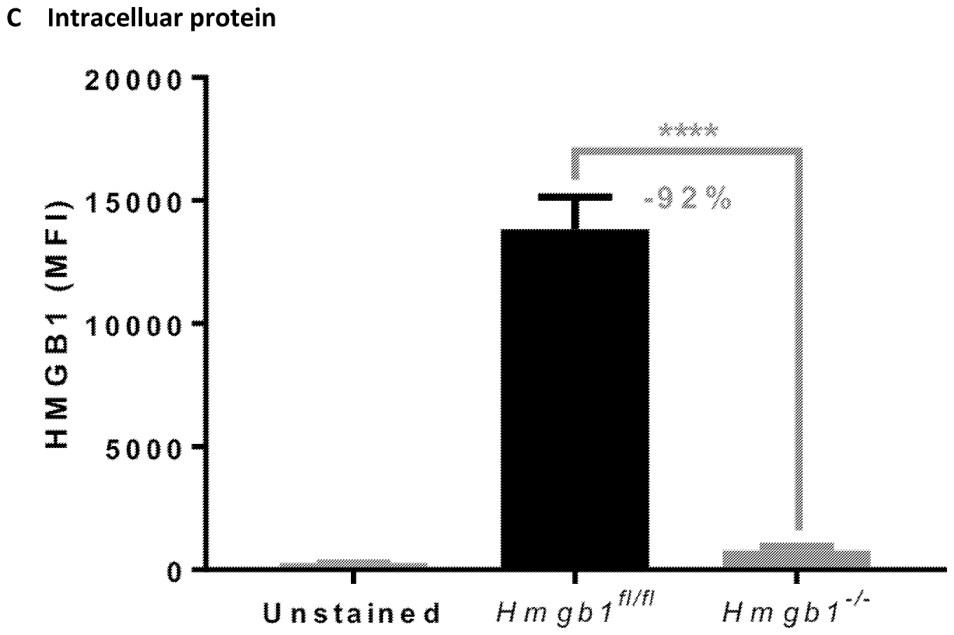

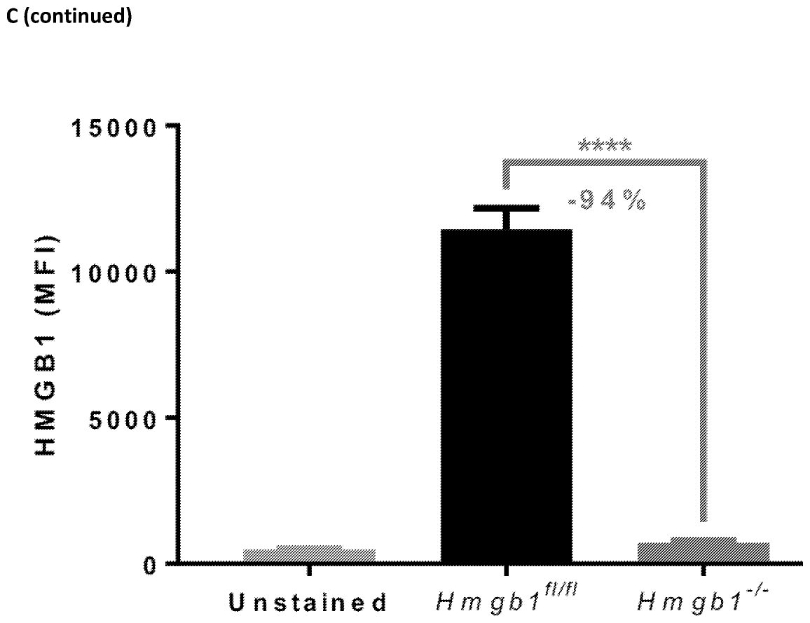

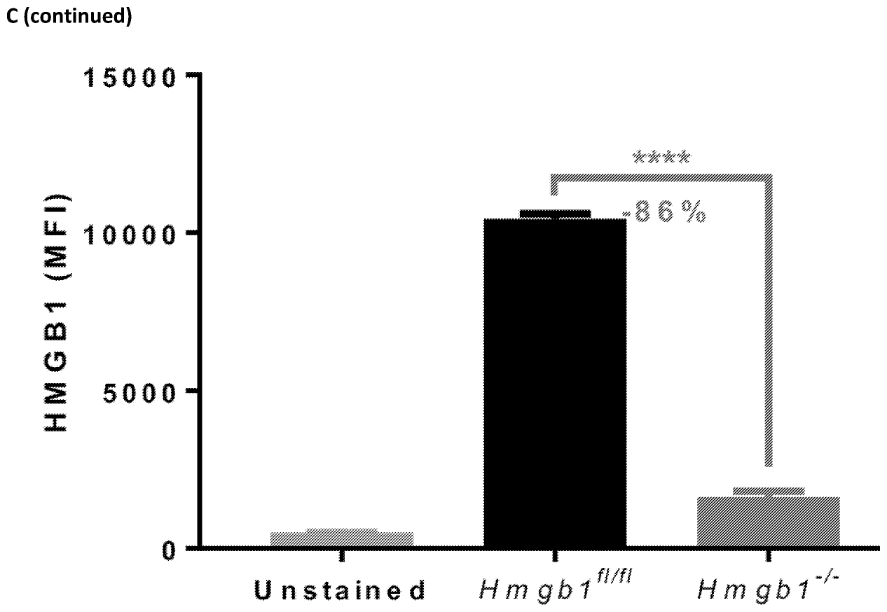

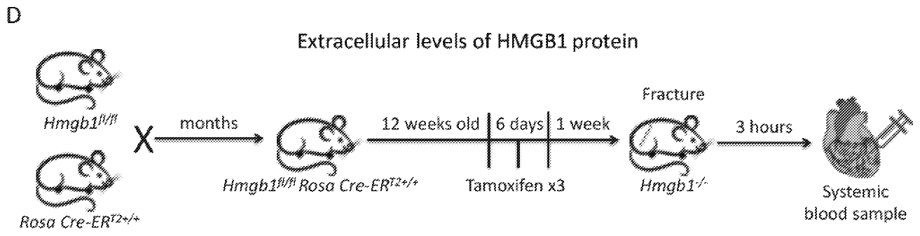

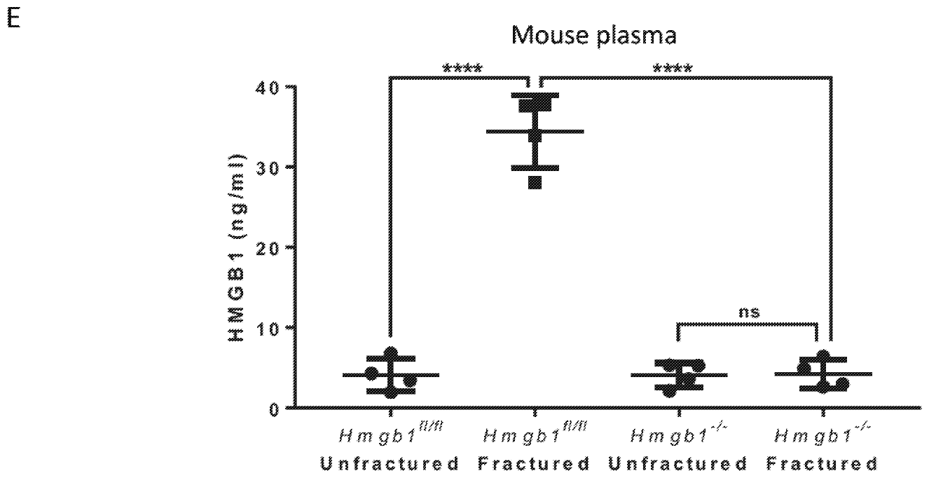

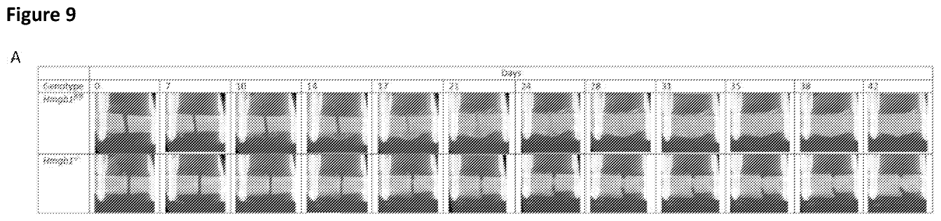

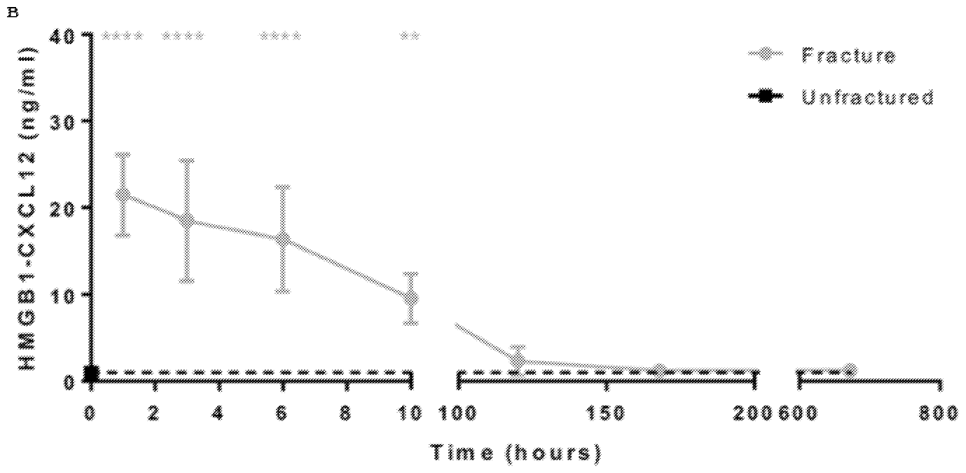

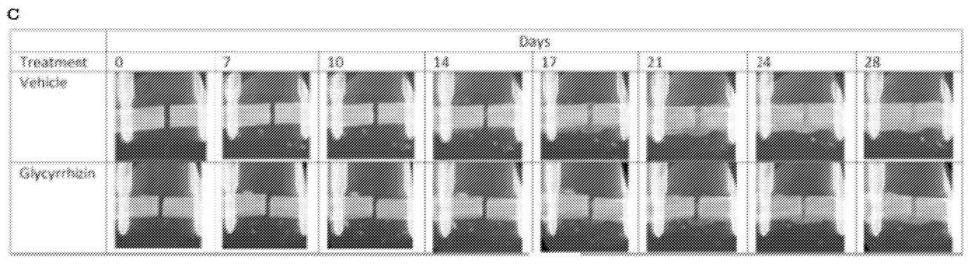

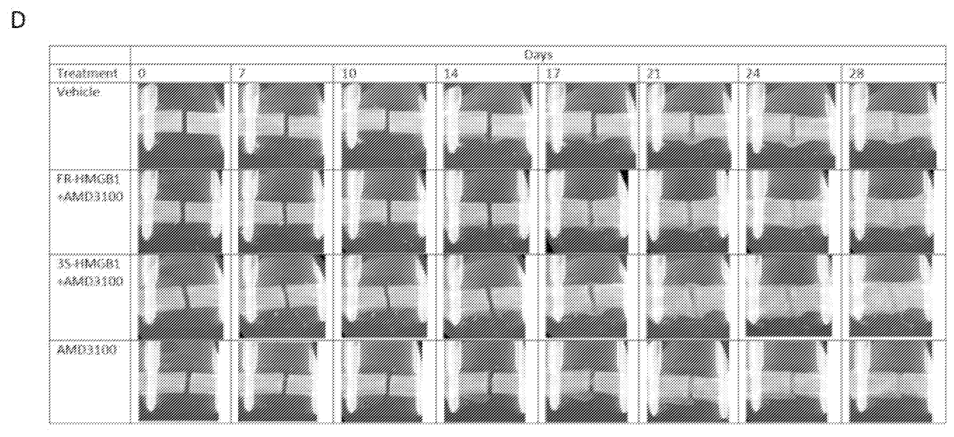

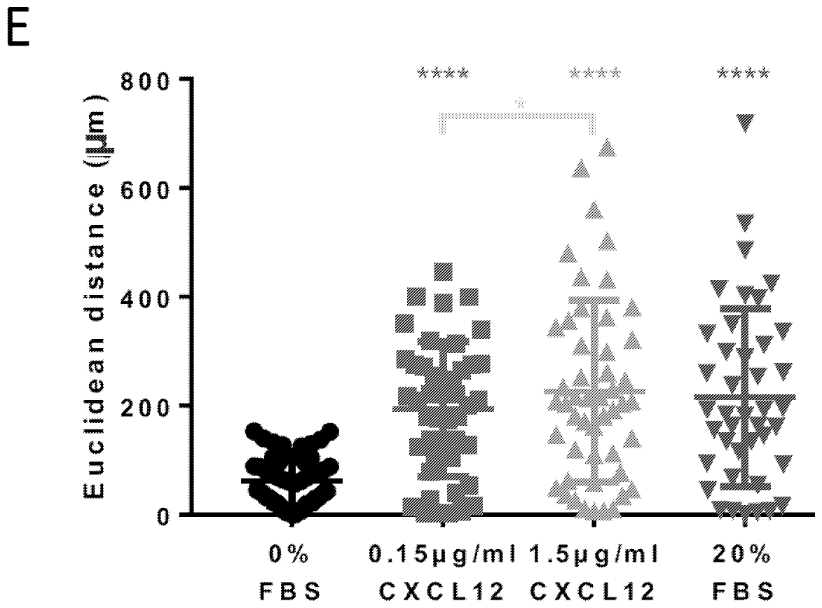

A murine fracture model (Zwingenberger 2013) was optimized to permit longitudinal in vivo analysis over time (FIG. 7 A-G) and it was found that FR or 3S-HMGB1 administered locally at the time of injury accelerated fracture repair as evidenced by in vivo microCT and mechanical strength testing (FIGS. 2A and B), with a clear dose-response (FIG. 7H). To evaluate the contribution of endogenous HMGB1 to fracture healing, inducible whole body Hmgb1^{-/-} mice were generated (FIG. 8) as FR-HMGB1 in the fracture microenvironment would originate from multiple injured and activated cell types, and constitutive deletion of HMGB1 is perinatally lethal (Yanai 2013). Fracture healing was dramatically impaired in these animals as shown by reduced callus volume, callus BMD and mechanical strength (FIG. 2C and FIG. 9A). Thus, both exogenous and endogenous HMGB1 modulate the rate of fracture healing.

### HMGB1 Accelerates Fracture Healing Via CXCL12 and CXCR4.

Subsequently, the signaling pathways through which HMGB1 promoted regeneration were delineated. FR-HMGB1 is known to form a heterocomplex with CXCL12 (Venereau 2012, Schiraldi 2012), a chemokine, which in turn binds to the receptor, CXCR4 (Venereau 2012, Schiraldi 2012). Elevated plasma levels of the HMGB1-CXCL12 heterocomplex were found in both human patients and mice following fracture injury (FIGS. 2 D and E, and FIG. 9B). Glycyrrhizin is the only known inhibitor of the HMGB1-CXCL12 heterocomplex (Schiraldi 2012). It interacts with the binding sites of HMGB1 for CXCL12 but not those for RAGE on the Box regions of HMGB1 Schiraldi 2012(27-29), thereby inhibiting the chemotactic activity of the heterocomplex in vitro and in vivo (Schiraldi 2012, Mollica 2007). Local administration of glycyrrhizin at the fracture site inhibited formation of the HMGB1-CXCL12 heterocomplex (FIG. 2E) and resulted in delayed fracture healing (FIG. 2F and FIG. 9C), confirming that endogenous extracellular HMGB1 modulates the rate of regeneration by forming a heterocomplex with CXCL12. Murine skeletal stem cells (Chan CKF 2015) (mSSC) were shown to express functional CXCR4 (FIGS. 2 H and I, and FIG. 9E) and administration of AMD3100, a specific and clinically approved small molecule inhibitor of CXCR4, led to impaired fracture healing in wild type mice (FIG. 2G and FIG. 9D), and completely abolished the effects of exogenous HMGB1 (FIG. 2G and FIG. 9D). These data confirm that exogenous HMGB1 accelerates tissue regeneration through CXCR4. The HMGB1-CXCL12 heterocomplex causes a conformational change in CXCR4 that is different compared to CXCL12 alone, and thereby enhances chemotaxis compared to CXCL12 (Schiraldi 2012). It was possible that the pro-regenerative effects of HMGB1 were simply due to enhanced CXCL12-mediated chemotaxis. To test this, we administered exogenous CXCL12 alone, and whilst we confirmed enhanced migration of cells to the fracture site (FIG. 7L), we only found abnormal regeneration as evidenced by a larger fracture callus without a concomitant increase in bone mineral density or, importantly, mechanical strength (FIGS. 2 A and B). Therefore, the improved regenerative effects of FR or 3S-HMGB1 could not have been due to enhanced CXCL12-mediated cell migration alone. Taken together, these data show that whilst the CXCL12-CXCR4 axis is necessary for HMGB1-mediated accelerated tissue regeneration, exogenous CXCL12 alone is insufficient to accelerate fracture healing. This suggests that the HMGB1-CXCL12 heterocomplex accelerates regeneration via an as yet unknown mechanism, rather than enhanced chemotaxis alone.

Exogenous HMGB1 led to a sustained increase in mSSC cell cycling in vivo.

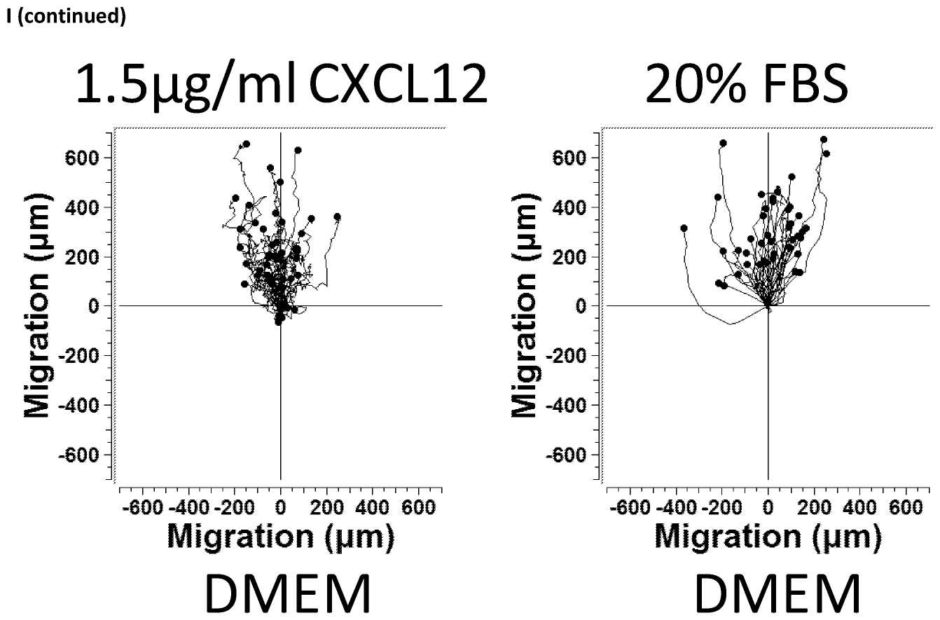

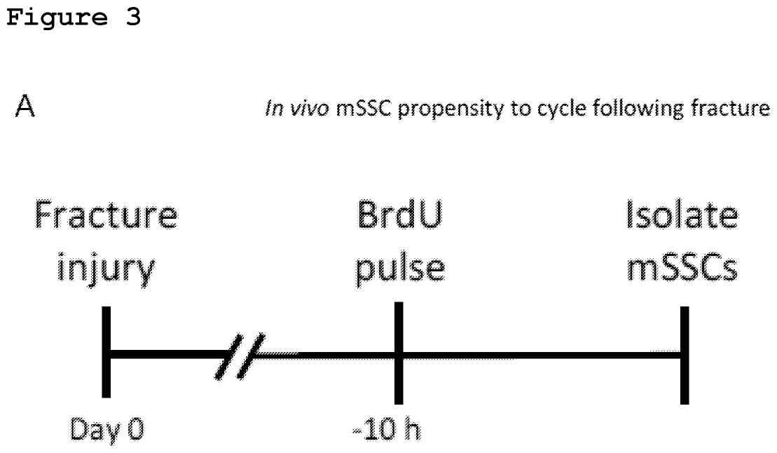

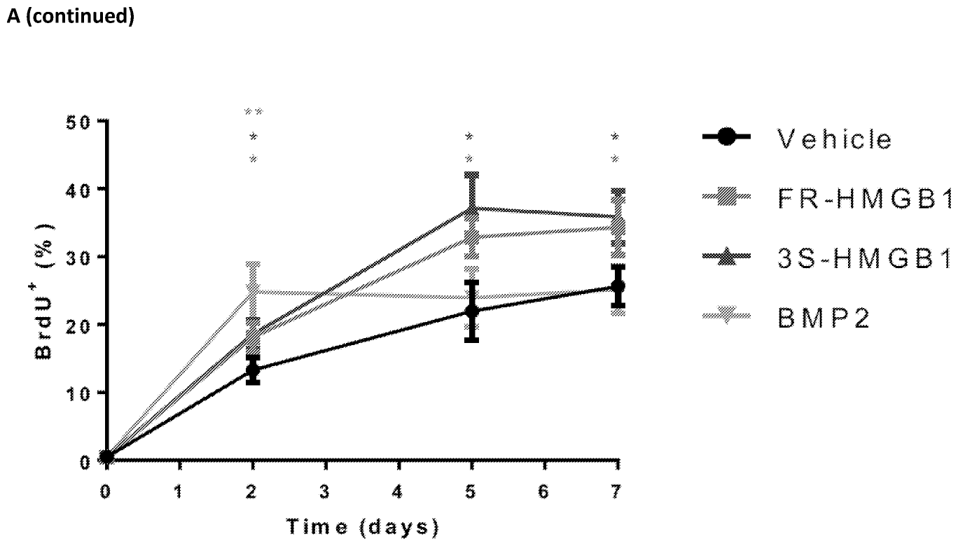

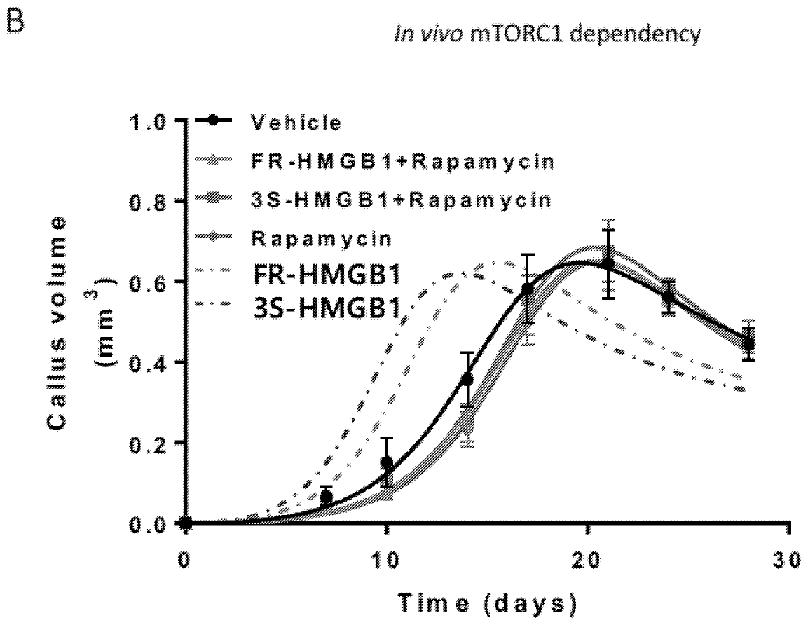

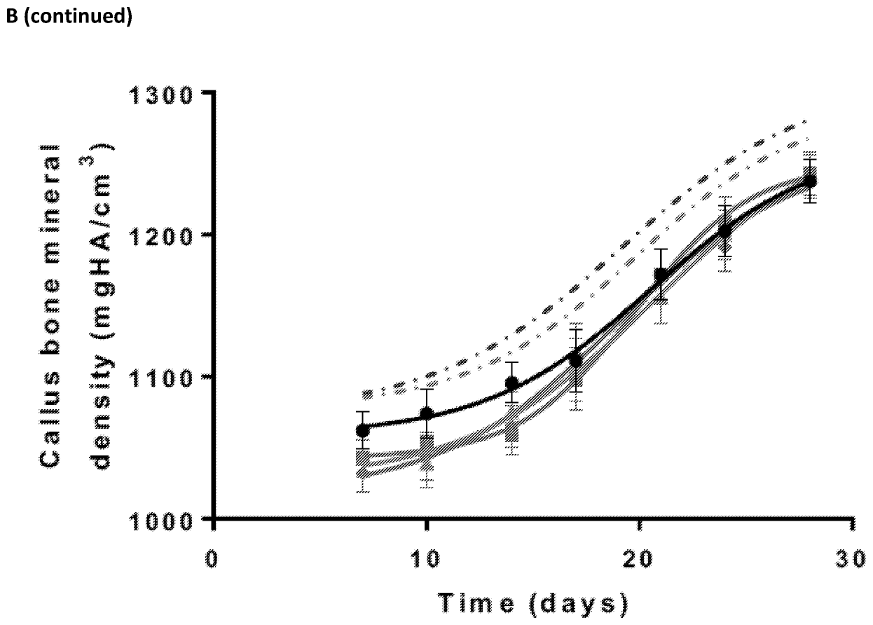

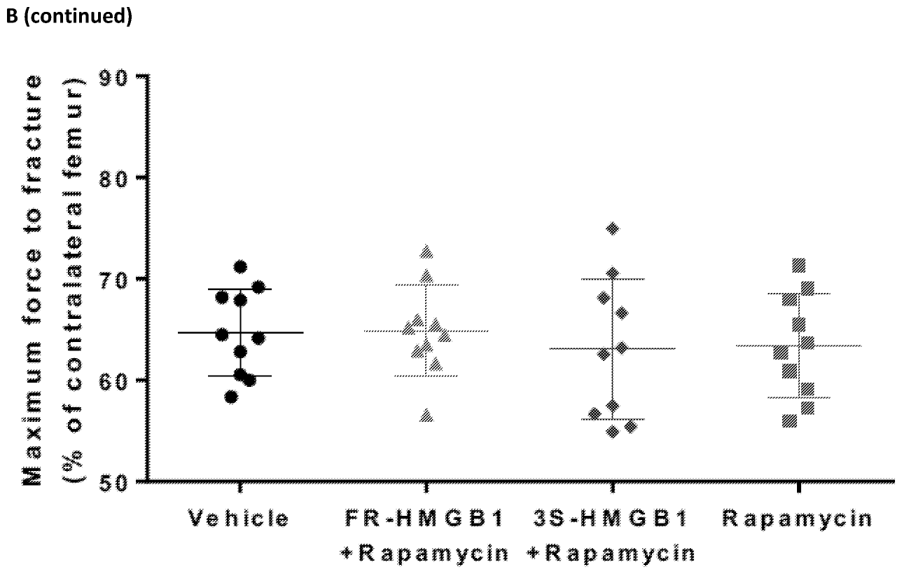

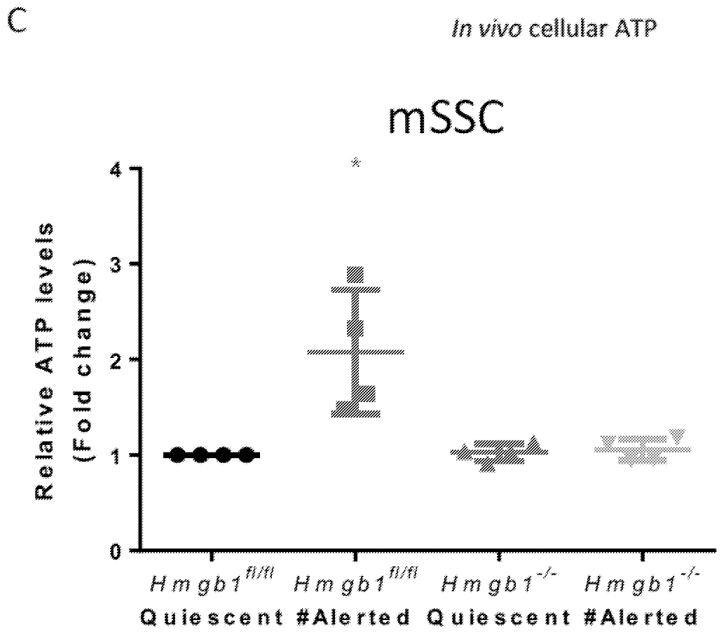

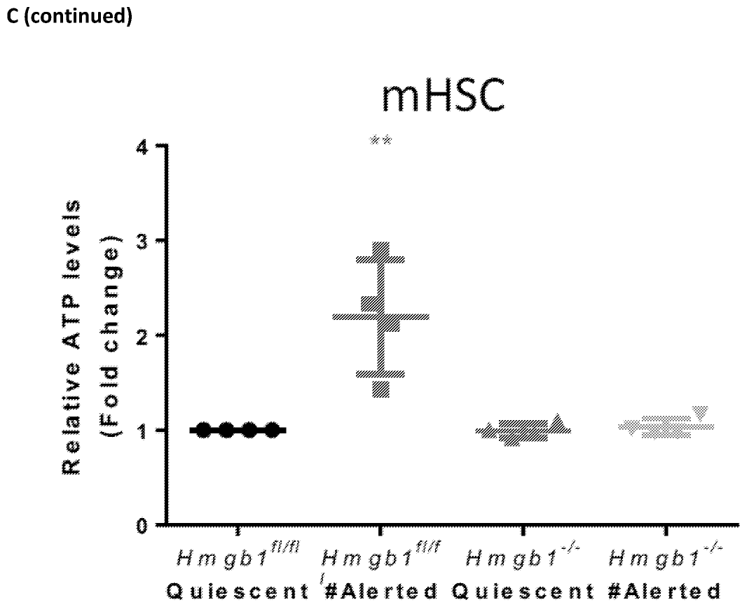

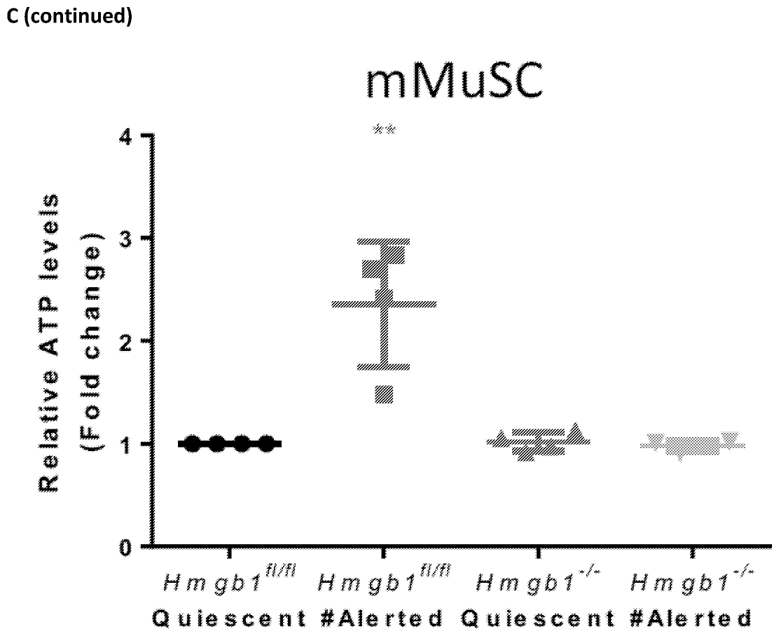

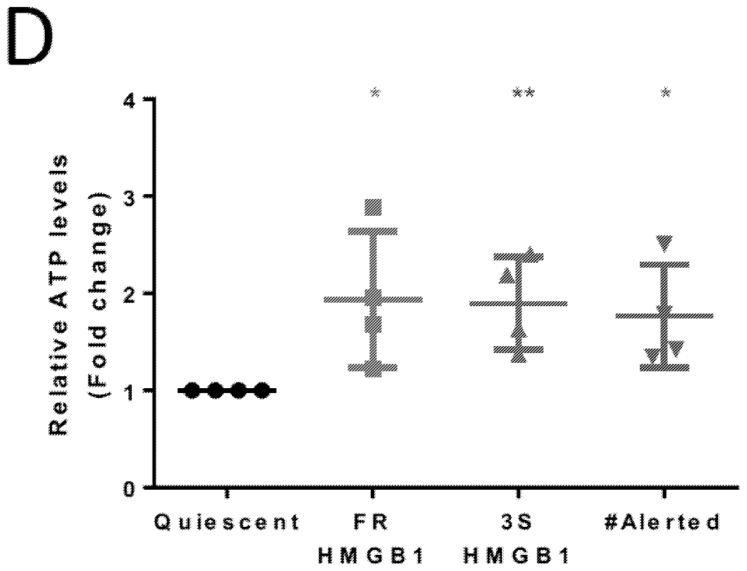

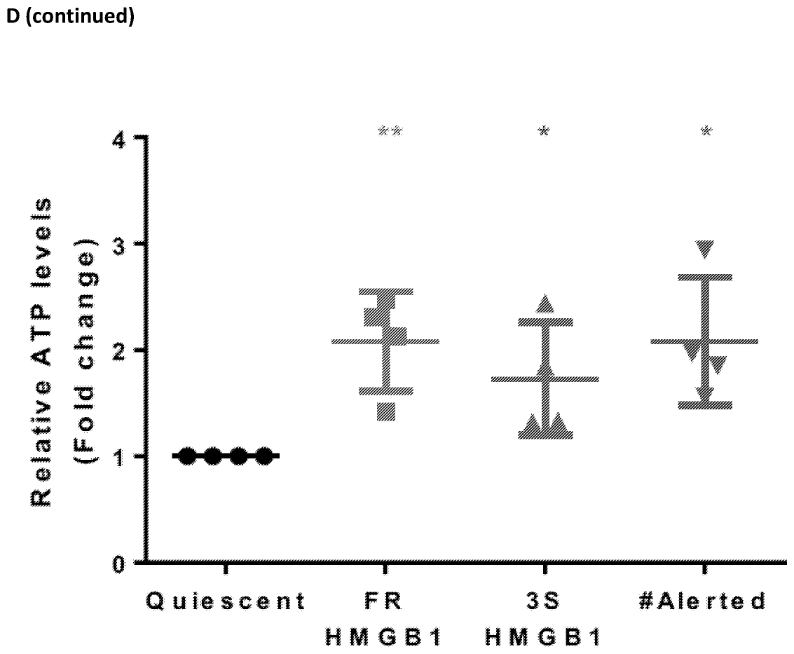

Apart from regulating chemotaxis, the CXCL12-CXCR4 axis also influences the cycling of haematopoietic stem cells by enforcing quiescence (Peled 1999, Sugiyama 2006, Nie 2008, Tzeng 2011, Ding 2013, Greenbaum 2013). Therefore, whether the HMGB1-CXCL12-CXCR4 axis additionally affects the cell cycle of stem cells to promote tissue regeneration was investigated. The propensity to cycle of mSSCs from the fractured bones of mice that had been pulse-labelled with BrdU (FIG. 3A) was analyzed. Murine SSCs from vehicle-treated animals displayed an increasing propensity to cycle over time, which correlates with the known rising levels post fracture of osteogenic mediators (Cho 2002, Einhorn 2015), including Bone Marrow Proteins (BMPs) (Chan 2015). Predictably, exogenous administration of BMP2, a known activator of mSSCs (Chan CRF 2015), resulted in an immediate increased propensity to cycle that plateaued at day 2 to levels equivalent to vehicle controls at day 5. In comparison, mSSCs from animals treated locally with exogenous FR or 3S-HMGB1 showed an initial increase intermediate between BMP2 and vehicle controls, and beyond day 2 exhibited a higher rate of cycling than cells from BMP2 or vehicle-treated animals. These data suggest that HMGB1 has an effect markedly different from an activator such as BMP2—cells that have been pre-exposed to HMGB1 display an increased propensity to cycle when subsequently exposed to endogenous activating signals released at the fracture site, indicative of a lasting cellular effect that favours cell cycle entry.

### HMGB1 Transitions Multiple Human and Murine Stem and Progenitor Cells to G_{Alert}.

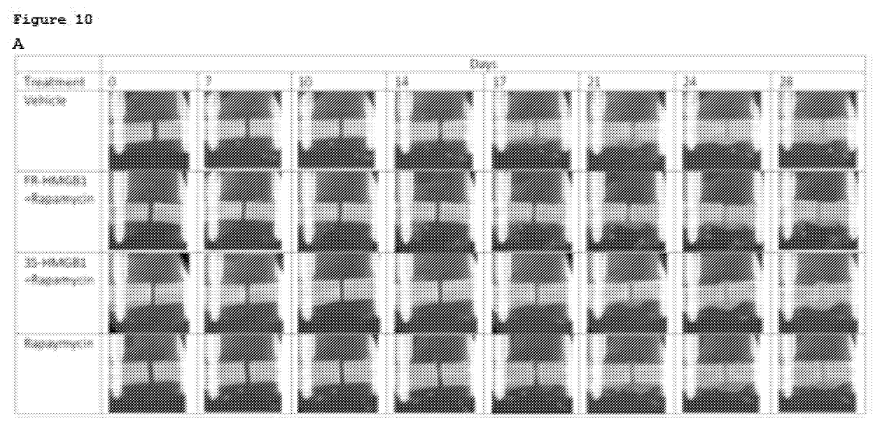

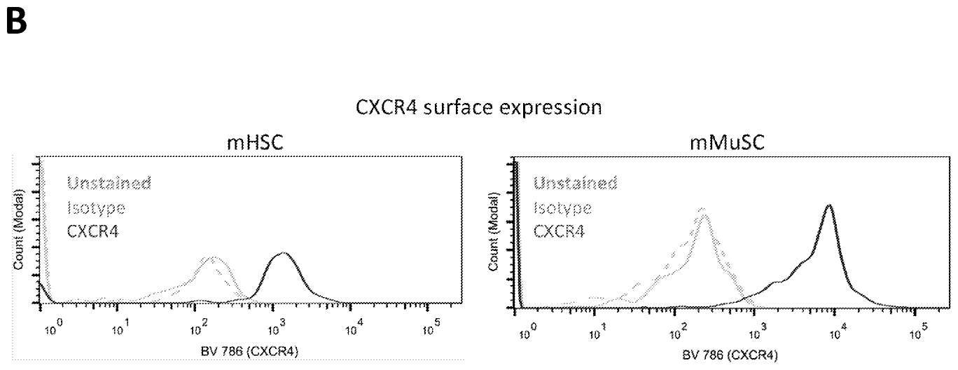

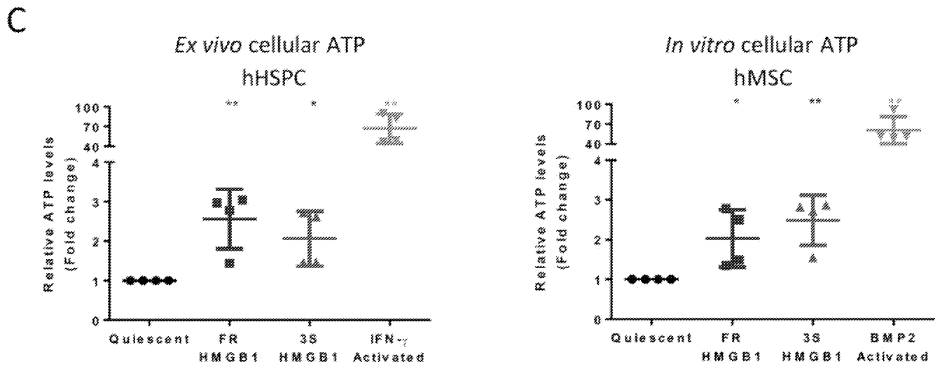

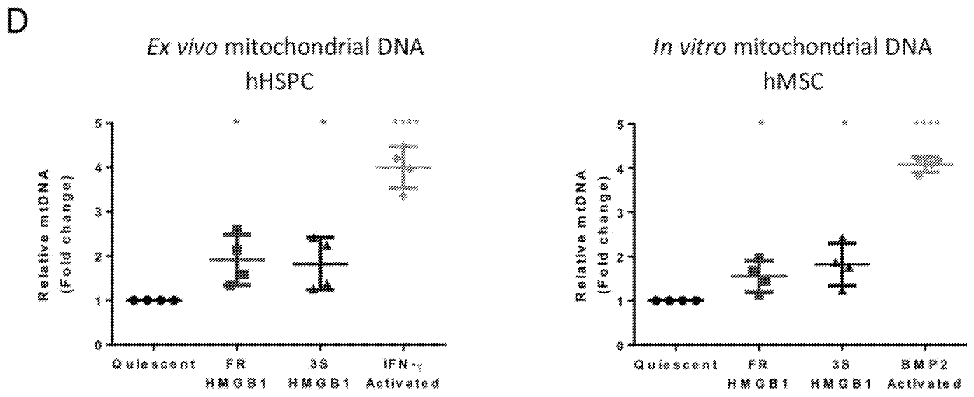

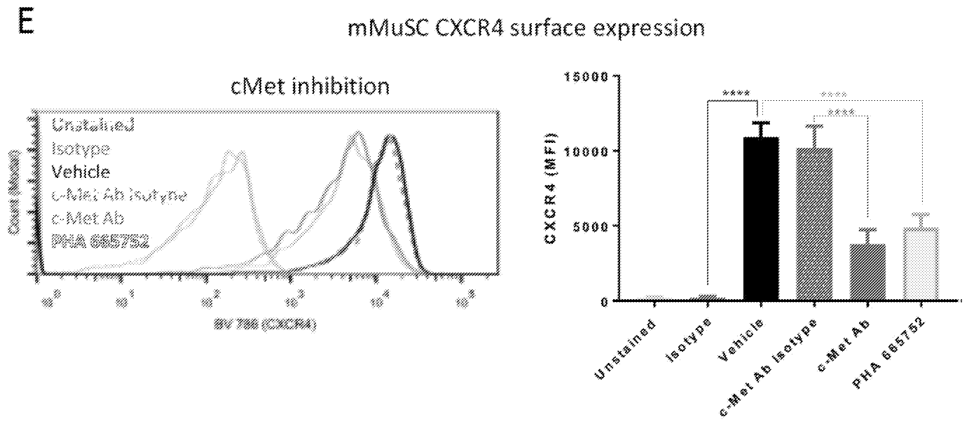

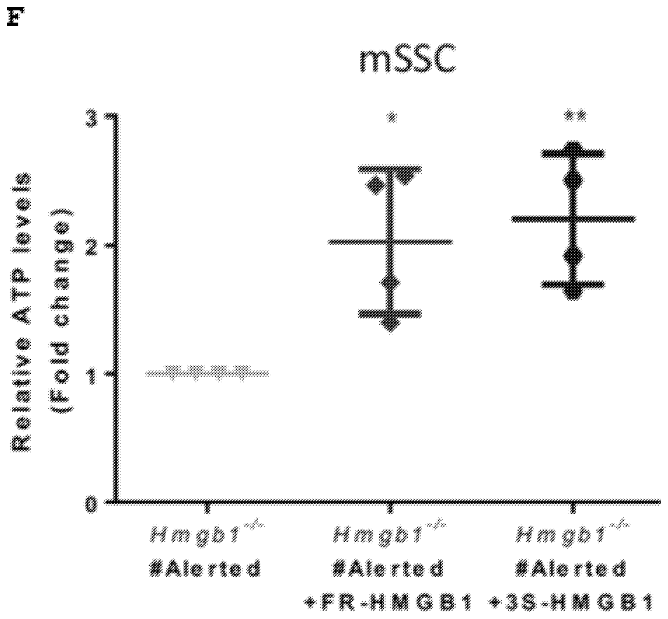

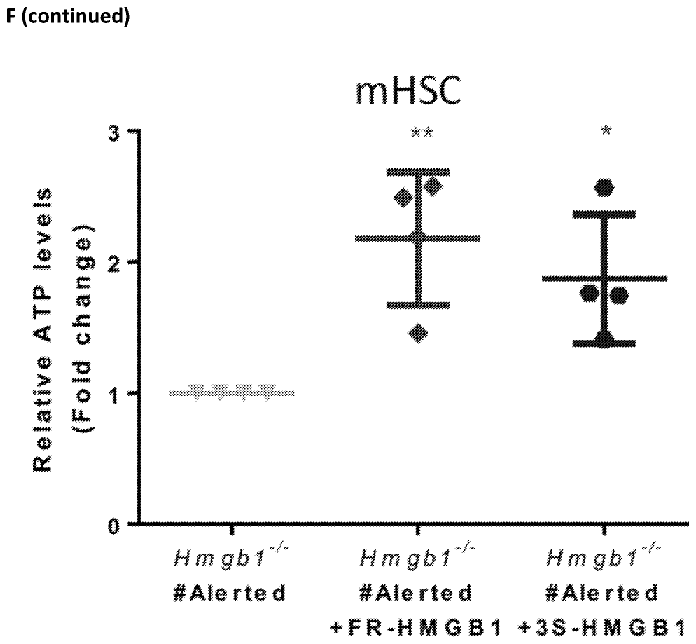

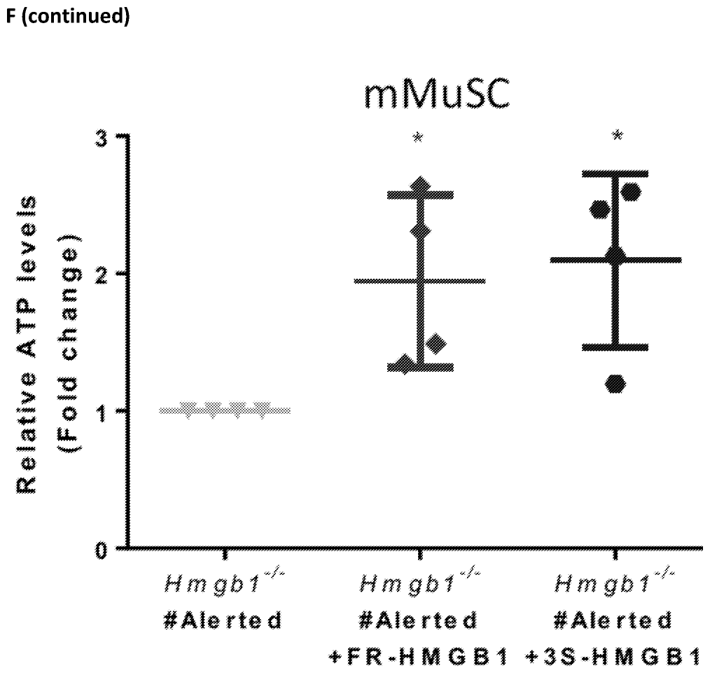

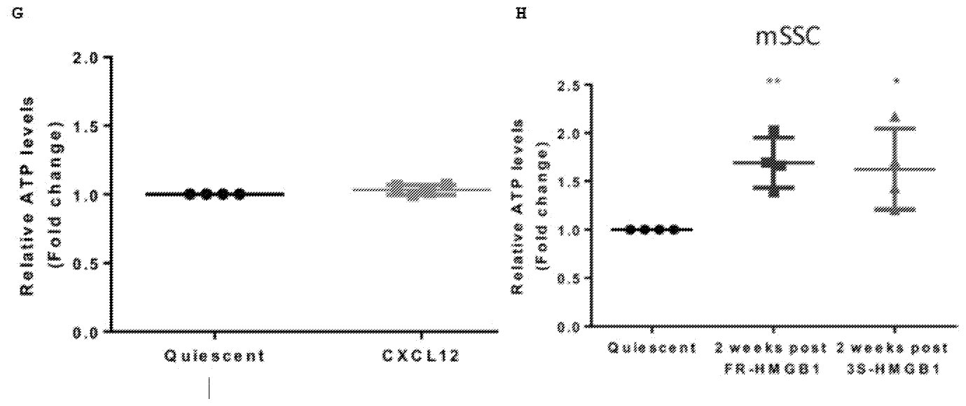

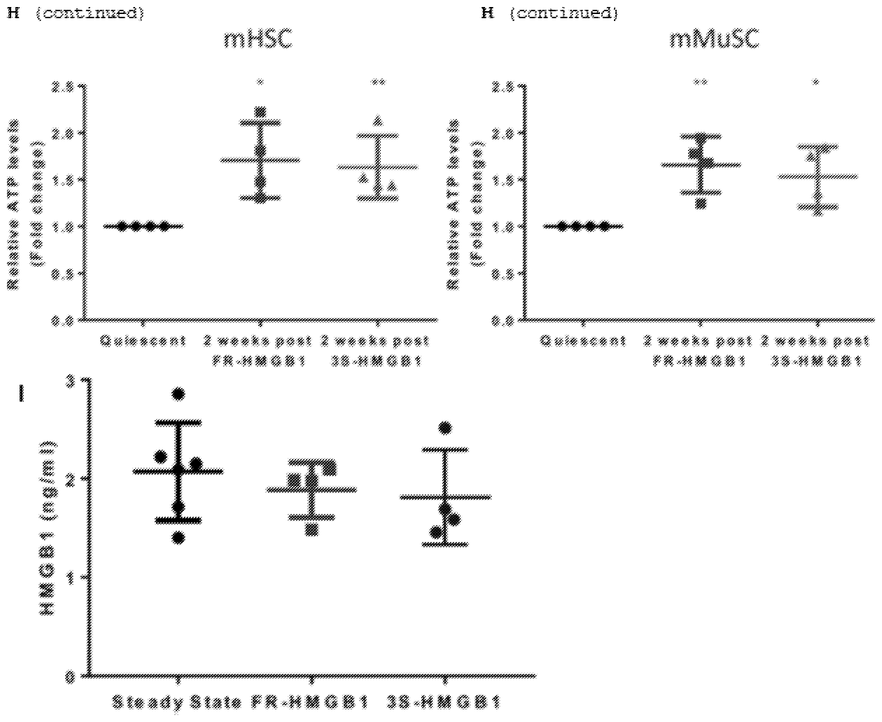

An elegant series of experiments recently demonstrated that systemic mediator(s) can transition stem cells distant to the site of initial injury to a dynamic state of the cell cycle, intermediate between G₀ and G₁, termed G_{Alert} (Rodgers 2014). In contrast to deeply quiescent G₀ stem cells, G_{Alert} cells are more metabolically active as evidenced by increased cellular levels of ATP and are poised to enter the cell cycle when exposed to activating signals. As HMGB1 enhanced the in vivo cycling of mSSCs exposed to secondary activating signals, together with the elevated systemic levels of HMGB1 and HMGB1-CXCL12 post-injury in humans and mice, and observations of accelerated fracture healing with exogenous HMGB1 treatment, it is hypothesized that HMGB1 may in part accelerate fracture healing by transitioning mSSCs to the recently defined G_{Alert} state. It is also postulated that these effects may pertain to other previously well-identified and characterized stem cells known to express CXCR4, including murine haematopoietic (mHSCs) (Peled 1999, Sugiyama 2006, Nie 2008, Tzeng 2011, Ding 2013, Greenbaum 2013) and muscle stem cells (mMuSCs) (Maesner 2016) (FIG. 10B).

The essential criteria describing the G_{Alert} state are increased ATP levels, mitochondrial DNA, cell size, faster entry to cell cycle, and mTORC1 dependency (Rodgers 2014). We found that the clinically approved mTORC1 inhibitor, rapamycin, abolished the accelerated healing effects of exogenous HMGB1 (FIG. 3B and FIG. 10A). To investigate the other aspects of the G_{Alert} state, we compared the cells contralateral to a fracture injury (fracture (#) alerted) to those from mice injected intravenously with HMGB1, or vehicle control. The severity of injury is important as only substantial injuries, such as fractures, can transition stem cells to G_{Alert}, whereas simple venepuncture is insufficient (Rodgers 2014). We observed that not only mSSCs, but also mHSCs, and mMuSCs from uninjured mice injected systemically with HMGB1 showed increased ATP levels, mitochondrial DNA, and cell size, compared to vehicle treated controls, and equivalent to fracture-alerted stem cells (FIG. 3 D-F). In contrast, stem cells from fractured Hmgb1^{-/-} mice (FIG. 3C) and SSCs from uninjured wild-type animals treated with CXCL12 did not transition to G_{Alert} (FIG. 10G). The essential role of exogenous HMGB1 was further confirmed with a single systemic dose of HMGB1 rescuing the elevated ATP G_{Alert} phenotype in stem cells from Hmgb1^{-/-} mice (FIG. 10F). The translational potential of the data herein is highlighted by the finding that HMGB1-treated human CD34+ hematopoietic stem and progenitor cells as well as MSCs exhibited increased ATP levels and mitochondrial DNA upon exposure to HMGB1 but substantially less so than IFN-γ (Baldridge 2010) or BMP2 activated cells, respectively (FIGS. 10 C and D). To assess the rate of entry into cell cycle in vivo, high-dose BrdU was continuously administered, thus utilizing the dual properties of BrdU to label cells that cycle whilst also acting as an injury signal that activates quiescent stem cells and recruits them into the cell cycle (Wilson 2008). It was found that the mSSCs, mHSCs and mMuSCs in HMGB1-treated mice entered the cell cycle faster with continuous high dose BrdU compared to vehicle-treated controls, but much more slowly than activated stem cells from the injured proximal hind limb of fractured animals (fracture (#) activated) (FIG. 3G). The previous genetic studies which demonstrated the necessity of cMet signalling for mMuSCs to transition to G_{Alert} (Rodgers 2014) recently led to the identification of HGF-A, an enzyme which activates HGF, a ligand for c-Met, as a stem cell alerting factor (Rodgers 2017). Consistent with the cMet genetic studies (Rodgers 2014), we found that in vivo cMet inhibition, with PHA 665752 or anti-cMet resulted in a substantially reduced expression of surface CXCR4 on mMuSCs (FIG. 10E). Therefore, it is possible that the cMet and CXCR4 pathways are complementary. Collectively, the data herein shows that HMGB1 transitions multiple stem cells to G_{Alert}, priming them to cycle quickly in response to activation signals.

### HMGB1 Accelerates Healing of Multiple Tissues, Even if Administered 2 Weeks Before Injury.

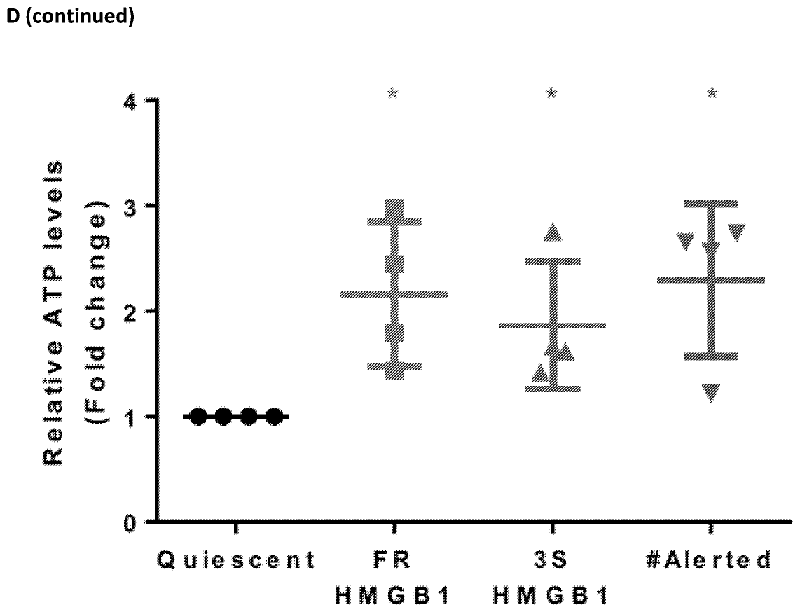

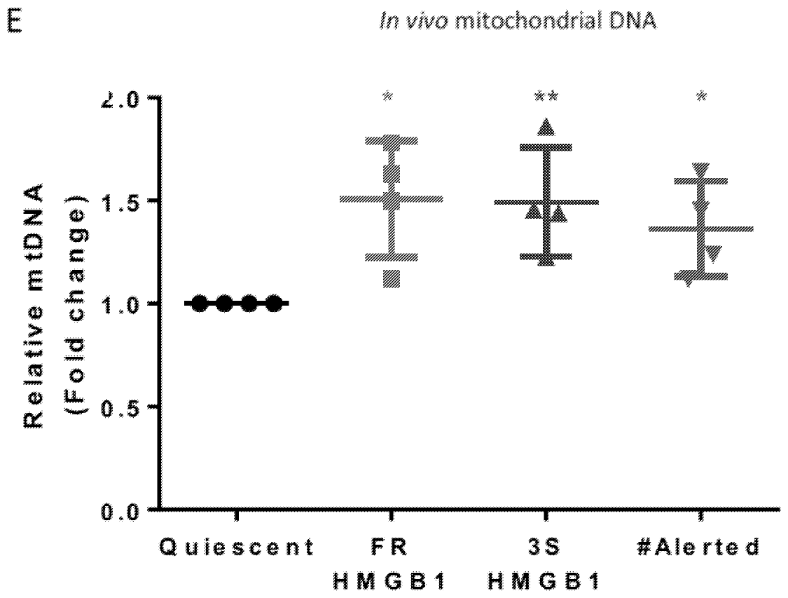

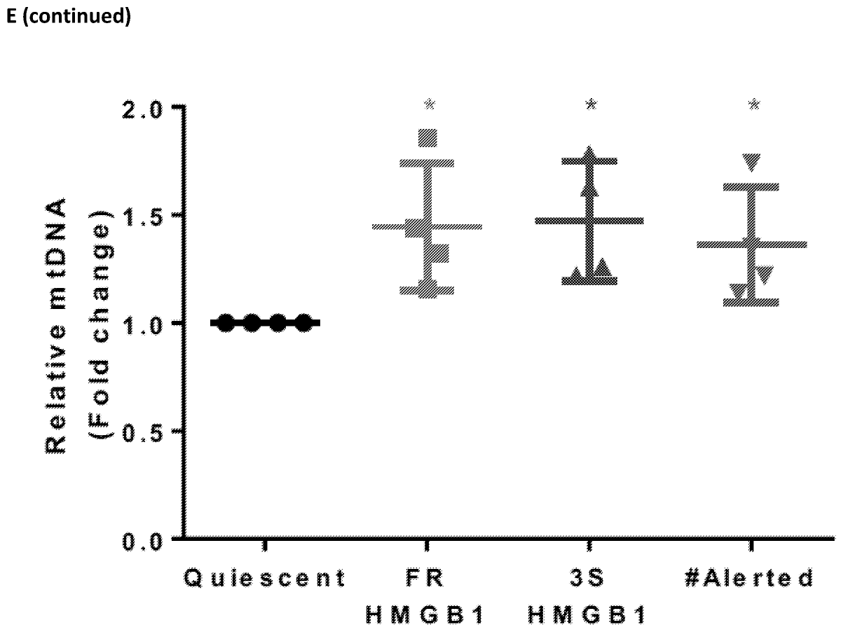

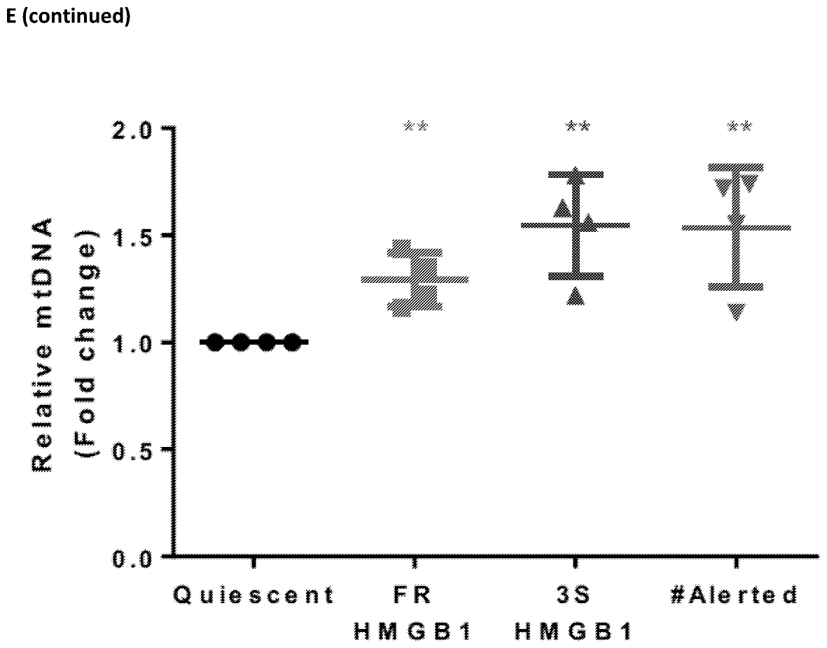

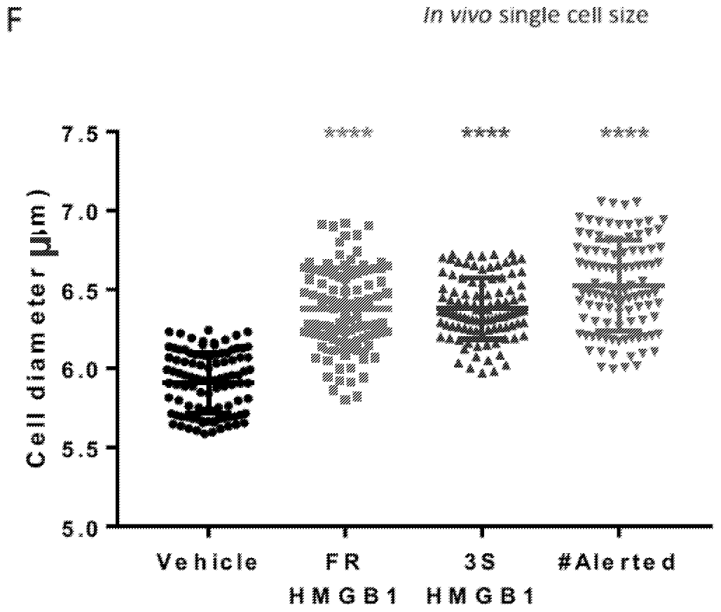

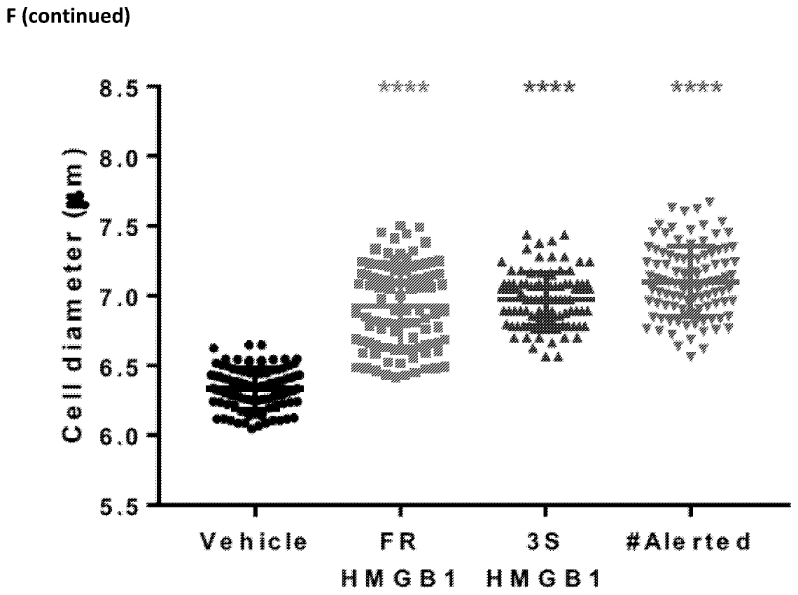

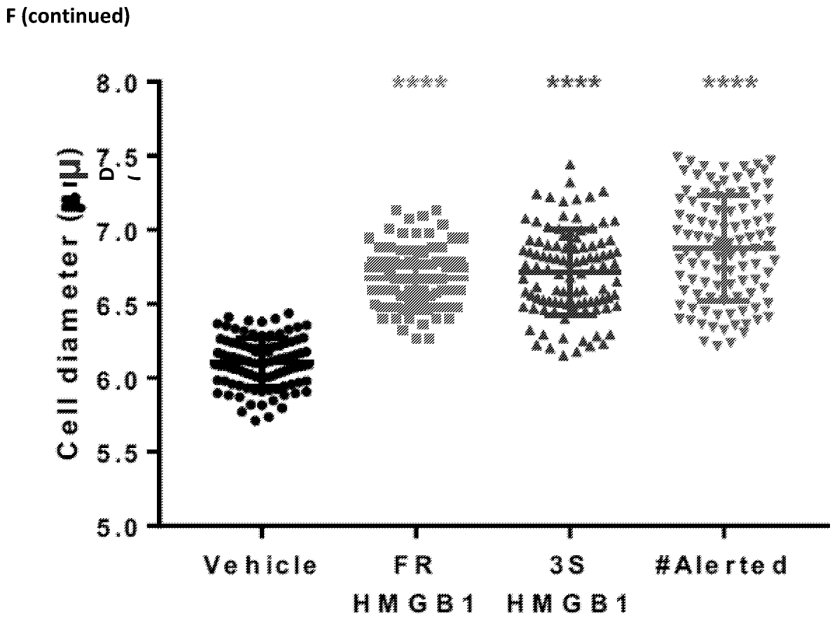

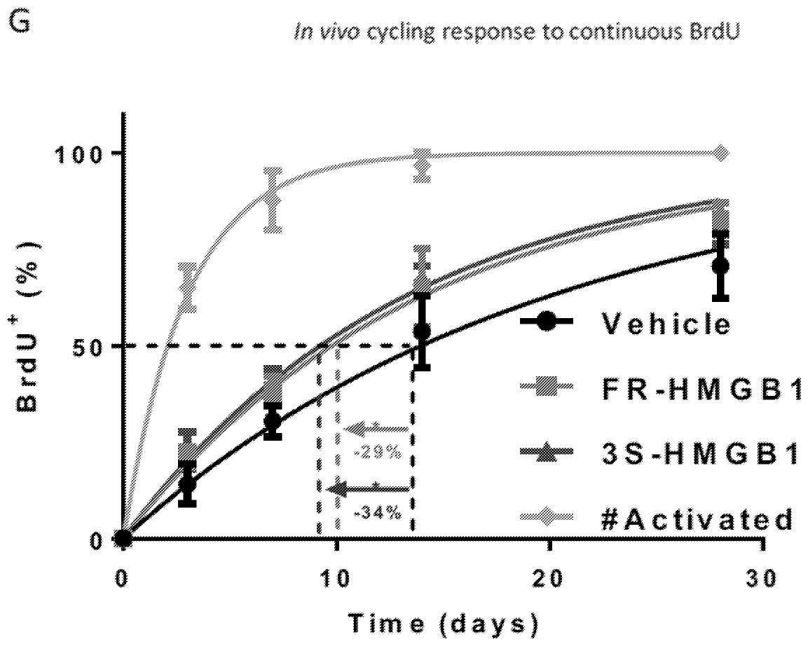

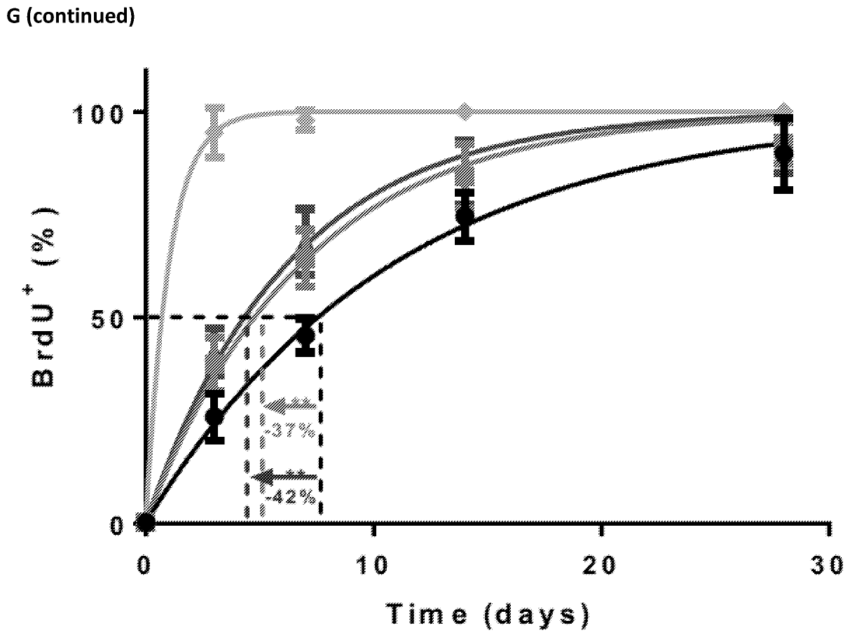

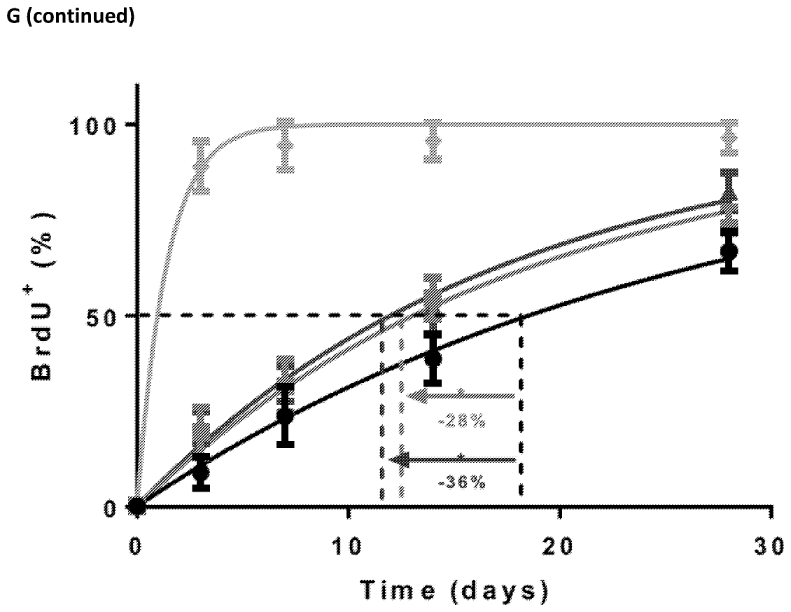

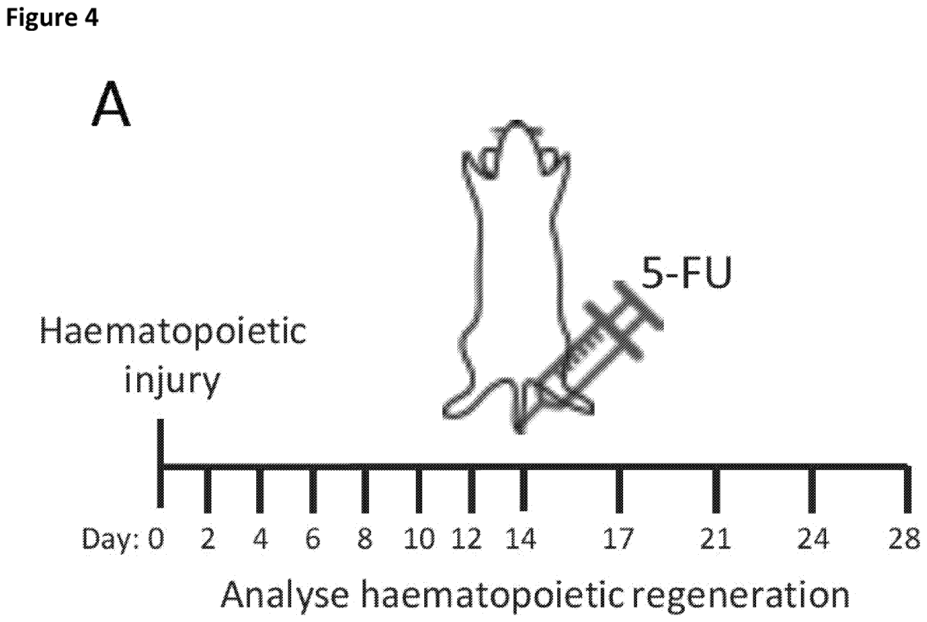

It was hypothesized that HMGB1 would also lead to accelerated tissue regeneration in other tissues where stem cells could transition to G_{Alert}, for example blood and muscle. In mice myeloablated with a common chemotherapeutic agent, 5-fluouracil (5-FU) (FIG. 4A), a single intravenous (i.v.) dose of HMGB1 at the time of injury led to accelerated recovery of systemic leucocyte (FIG. 4B) and neutrophil (FIG. 4C) counts. This has significant translational relevance because the duration of leucopenia and neutropenia is directly related to the risk of infection, with each day of neutropenia approximately doubling the risk of a febrile neutropenic episode (Bodey 1966). Febrile neutropenia is a medical emergency with a mortality rate of 6.8-9.5% (Lyman 2010), so accelerating hematopoietic recovery following chemotherapy would make chemotherapy safer for patients. It was also found that local administration of a single dose of HMGB1 at the time of injury resulted in accelerated muscle regeneration following BaCl₂ chemical injury (Rodgers 2014) (FIG. 4 D-F). Our finding that HMGB1 resulted in mSSCs having an increased propensity to cycle that is sustained for several days (FIG. 3A) is consistent with the previous observation that following injury, stem cells in the contralateral limb remain in G_{Alert} for 3-4 weeks (Rodgers 2014), and we found that 2 weeks post FR or 3S-HMGB1 administration, mSSCs, mHSCs, and mMuSCs still had elevated ATP (FIG. 10H) even though circulating levels of HMGB1 had already returned to baseline (FIG. 10I). Therefore, we investigated whether pre-treatment with a single i.v. dose of HMGB1, 2 weeks prior to injury would also accelerate bone, hematopoietic and muscle tissue regeneration. We observed accelerated tissue regeneration in all these tissues (FIG. 4 G-M). However, regeneration was only observed following injury, with no ectopic tissue formation in the 2 week period between HMGB1 treatment and injury. This indicates that HMGB1 treatment is a dynamic and adaptive form of multi-tissue regenerative therapy, which takes cues from the steady state or tissue-specific activating regenerative molecular signals present at that time. The pre-administration of HMGB1 would be particularly relevant in situations of planned or expected injury, including elective surgery, sports medicine or military combat.

### DISCUSSION

HMGB1 has been identified as a therapeutic target that acts on multiple endogenous adult stem cells to accelerate the physiological regenerative response to current or future injuries. These findings have broad relevance to the fields of stem cell biology and regenerative medicine and suggest a novel therapeutic approach to promote tissue repair. The existence of the G_{Alert} phase, which is intermediate between G₀ and G₁, was described previously (Rodgers 2014). It was noted that stem cells in G_{Alert}. enter the cell cycle faster compared to those in G₀ and initiators of this transition would have wide-ranging implications for the field of regenerative medicine by accelerating repair.

HMGB1 has been demonstrated to accelerate healing of multiple tissue types by forming a heterocomplex with CXCL12, which then binds to CXCR4, to transition quiescent stem cells in three different tissues to G_{Alert}. A recent publication (Tirone 2018) showed that HMGB1 promotes repair in a murine model of muscle injury in part by modulating the immune response. We utilized prospective multi-parameter flow cytometry isolation methodologies to study the cycling of well-defined endogenous adult stem cell populations in vivo to reduce potential in vitro artefacts and identified a novel mechanism of action of FR-HMGB1 during tissue repair via the initiation of the G_{Alert} state. Furthermore, we demonstrated that this also pertains to human stem and progenitor cells.

Whilst this work has focused on endogenous adult stem cells, it is possible that the transition to G_{Alert} by HMGB1 may also pertain to other cell types that are usually quiescent in the steady state, can express CXCR4 and are capable of re-entering the cell cycle to effect tissue repair, such as mature hepatocytes. Indeed, it was recently observed that HMGB1 treatment results in enhanced proliferation of hepatocytes following injury, although there was no concomitant improvement in liver function as evidenced by accelerated return of damage-associated liver enzymes to basal levels (Tirone 2018). Using clinically relevant injury models of fracture repair, the response to chemotherapy and muscle regeneration, in conjunction with human tissues and cells, applicants have demonstrated that FR-HMGB1 leads to accelerated regeneration of multiple tissues by transitioning the respective stem cells to G_{Alert}.

HMGB1 has critical intracellular and extracellular functions as demonstrated by the lethality of the constitutive global knockout (Kang 2014). In the nucleus HMGB1 interacts with nucleosomes, transcription factors and histones and thus regulates gene transcription. It has recently been shown that muscle regeneration is compromised in partial Hmgb1^{+/-} mice (Tirone 2018). Fracture healing has been shown to be dramatically impaired in conditional Hmgb1^{-/-} with robust intracellular and extracellular protein knockdown, and that stem cells fail to transition to G_{Alert}. At the cellular level, exogenous HMGB1 can rescue the G_{Alert} phenotype but did not evaluate the rescue at tissue healing level as exogenous HMGB1 addition would not compensate for the critical intra-nuclear roles of HMGB1 (Kang 2014).

Whilst extracellular FR-HMGB1 enhances cell migration by forming a heterocomplex with the relatively abundant CXCL12 that is produced following injury, our data shows that the enhanced regenerative effects of the heterocomplex extend beyond those explained by increased chemotaxis. Indeed, the novel finding that systemic pre-treatment with HMGB1 two weeks prior to injury also accelerates tissue regeneration, with stem cells remaining in G_{Alert} at this time point (FIG. 10H) despite no extracellular HMGB1 being detectable systemically to mediate chemotaxis or other processes (FIG. 10I), suggests that the cellular transition to G_{Alert} is a central mechanism of the accelerated repair process. This finding also expands the use of HMGB1 into the contexts of planned or expected potential injury, such as in sports medicine, military combat and elective surgery. The last is an area of urgent unmet medical need as each person in the USA undergoes on average 9.2 surgical procedures in their lifetime (Lee 2017).

HMGB1 is a pleotropic factor, with contrasting effects depending on the redox status. The in vitro screen confirmed that only priming of human bone-marrow derived MSC by FR or 3S-HMGB1 promoted osteogenesis on subsequent exposure to osteogenic factors. It was not found that exogenous administration of the FR-HMGB1 either locally or systemically resulted in any untoward inflammation, suggesting that potential conversion to the proinflammatory disulfide form may not be a limitation when considering development of a therapeutic. Furthermore, significant difference in the regenerative effects of 3S compared to FR-HMGB1 was not observed.

In summary, a major discovery of recent decades has been the existence of stem cells and their potential to repair many, if not most, tissues. With the aging population, many attempts have been made to use exogenous stem cells to promote tissue repair, so far with limited success. An alternative approach, which may be more effective and far less costly, is to promote tissue regeneration by targeting endogenous stem cells. However, ways of enhancing endogenous stem cell function remain poorly defined. Injury leads to the release of danger signals which are known to modulate the immune response, but their role in stem cell-mediated repair in vivo remains to be clarified. In this application it has demonstrated that high mobility Q:9 group box 1 (HMGB1) is released following fracture in both humans and mice, forms a heterocomplex with CXCL12, and acts via CXCR4 to accelerate skeletal, hematopoietic, and muscle regeneration in vivo. Pretreatment with HMGB1 2 weeks before injury also accelerated tissue regeneration, indicating an acquired proregenerative signature. HMGB1 led to sustained increase in cell cycling in vivo, and using Hmgb1^{-/-} mice we identified the underlying mechanism as the transition of multiple quiescent stem cells from G₀ to G_{Alert}. HMGB1 also transitions human stem and progenitor cells to G_{Alert}. Therefore, exogenous HMGB1 benefits patients in many clinical scenarios, including trauma, chemotherapy, and elective surgery.

This invention is significant because while stem cell therapy has become the standard of care for hematological disorders, challenges remain for the treatment of solid organ injuries. Targeting endogenous cells would overcome many hurdles associated with exogenous stem cell therapy. Alarmins are released upon tissue damage, and here it is described how upregulation of a physiological pathway by exogenous administration of a single dose of HMGB1, either locally or systemically, promotes tissue repair by targeting endogenous stem cells. It is shown that HMGB1 complexed with CXCL12 transitions stem cells that express CXCR4 from G₀ to G_{Alert}. These primed cells rapidly respond to appropriate activating factors released upon injury. HMGB1 promotes healing even if administered 2 weeks before injury, thereby expanding its translational benefit for diverse clinical scenarios.

### Example 2

A model is developed in which a highly-conserved injury signal, HMGB1, acts via a well-established maintenance signaling pathway, CXCL12-CXCR4, to promote tissue regeneration as depicted in FIG. 4N. This pathway is targeted to accelerate healing in any tissue that relies on repair by cells that express CXCR4 and can transition to G_{Alert}. FR-HMGB1 is administered as a single dose either locally or systemically soon after injury or even up to 2 weeks before injury to accelerate healing. Administration up to 2 weeks before injury accelerates healing. Administration up to 3 weeks before injury also accelerates healing. Additionally, administration at the time of injury or soon after injury accelerates healing.

### REFERENCES

1. Weissman I L (2000) Stem cells: units of development, units of regeneration, and units in evolution. Cell 100(1):157-68.
2. Gratwohl A, et al. (2015) One million haemopoietic stem-cell transplants: a retrospective observational study. Lancet Haematol 2(3):e91-100.
3. Brooks M (2017) Stem cell research: time for a dose of realism. BMJ 356:j443.
4. Trainor N, Pietak A, Smith T (2014) Rethinking clinical delivery of adult stem cell therapies. Nat Biotechnol 32(8):729-735.
5. Dimmeler S, Ding S, Rando T A, Trounson A (2014) Translational strategies and challenges in regenerative medicine. Nat Med 20(8):814-21.
6. Forbes S J, Rosenthal N (2014) Preparing the ground for tissue regeneration: from mechanism to therapy. Nat Med 20(8):857-69.
7. Lane S W, Williams D A, Watt F M (2014) Modulating the stem cell niche for tissue regeneration. Nat Biotechnol 32(8):795-803.
8. Zhang Y, et al. (2015) Inhibition of the prostaglandin-degrading enzyme 15-PGDH potentiates tissue regeneration. Science 348(6240):2340-1-8.
9. Joseph J, Dyson M (1970) The effect of abdominal wounding on the rate of tissue regeneration. Experientia 26(1):66-67.
10. Davis T A, Longcor J D, Hicok K C, Lennon G G (2005) Prior injury accelerates subsequent wound closure in a mouse model of regeneration. Cell Tissue Res 320(3):417-426.
11. Rodgers J T, et al. (2014) mTORC1 controls the adaptive transition of quiescent stem cells from G0 to G(Alert). Nature 510(7505):393-6.
12. Harry L E, et al. (2008) Comparison of the healing of open tibial fractures covered with either muscle or fasciocutaneous tissue in a murine model. J Orthop Res 26(9):1238-1244.
13. Glass G E, et al. (2011) TNF-alpha promotes fracture repair by augmenting the recruitment and differentiation of muscle-derived stromal cells. Proc Natl Acad Sci USA 108(4):1585-90.
14. Chan J K, et al. (2015) Low-dose TNF augments fracture healing in normal and osteoporotic bone by up-regulating the innate immune response. EMBO Mol Med 7(5):547-561.
15. Chan J K, et al. (2012) Alarmins: awaiting a clinical response. J Clin Invest 122(8):2711-9.
16. Scaffidi P, Misteli T, Bianchi M E (2002) Release of chromatin protein HMGB1 by necrotic cells triggers inflammation. Nature 418(6894):191-5.
17. Terrando N, et al. (2010) Tumor necrosis factor-alpha triggers a cytokine cascade yielding postoperative cognitive decline. Proc Natl Acad Sci USA 107(47):20518-22.
18. Harris H E, Andersson U, Pisetsky D S (2012) EMGB1: a multifunctional alarmin driving autoimmune and inflammatory disease. Nat Rev Rheumatol 8(4):195-202.
19. Horiuchi T, et al. (2017) Metformin directly binds the alarmin HMGB1 and inhibits its proinflammatory activity. J Biol Chem 292(20):8436-8446.
20. Tirone M, et al. (2018) High mobility group box 1 orchestrates tissue regeneration via CXCR4. J Exp Med 215(1):303-318.
21. Einhorn T A, Gerstenfeld L C (2015) Fracture healing: mechanisms and interventions. Nat Rev Rheumatol 11(1):45-54.
22. Edgeworth J, Gorman M, Bennett R, Freemont P, Hogg N (1991) Identification of p8,14 as a highly abundant heterodimeric calcium binding protein complex of myeloid cells. J Biol Chem 266(12):7706-13.
23. Zreiqat H, Howlett C R, Gronthos S, Hume D, Geczy C L (2007) S100A8/S100A9 and their association with cartilage and bone. J Mol Histol 38(5):381-91.
24. Venereau E, et al. (2012) Mutually exclusive redox forms of HMGB1 promote cell recruitment or proinflammatory cytokine release. J Exp Med 209(9):1519-28.
25. Zwingenberger S, et al. (2013) Establishment of a femoral critical-size bone defect model in immunodeficient mice. J Surg Res 181 (1):e7-e14.
26. Yanai H, et al. (2013) Conditional ablation of HMGB1 in mice reveals its protective function against endotoxemia and bacterial infection. Proc Natl Acad Sci USA 110(51):20699-704.
27. Schiraldi M, et al. (2012) EMGB1 promotes recruitment of inflammatory cells to damaged tissues by forming a complex with CXCL12 and signaling via CXCR4. J Exp Med 209(3):551-63.
28. Mollica L, et al. (2007) Glycyrrhizin binds to high-mobility group box 1 protein and inhibits its cytokine activities. Chem Biol 14(4):431-41.
29. Huttunen H J, Fages C, Kuja-Panula J, Ridley A J, Rauvala H (2002) Receptor for advanced glycation end products-binding COOH-terminal motif of amphoterin inhibits invasive migration and metastasis. Cancer Res 62(16):4805-11.
30. Chan C K F, et al. (2015) Identification and Specification of the Mouse Skeletal Stem Cell. Cell 160(1-2):285-298.
31. Peled A, et al. (1999) Dependence of human stem cell engraftment and repopulation of NOD/SCID mice on CXCR4. Science (80-) 283(5403):845-8.
32. Sugiyama T, Kohara H, Noda M, Nagasawa T (2006) Maintenance of the Hematopoietic Stem Cell Pool by CXCL12-CXCR4 Chemokine Signaling in Bone Marrow Stromal Cell Niches. Immunity 25(6):977-988.
33. Nie Y, Han Y-C, Zou Y-R (2008) CXCR4 is required for the quiescence of primitive hematopoietic cells. J Exp Med 205(4):777-83.
34. Tzeng Y-S, et al. (2011) Loss of Cxc112/Sdf-1 in adult mice decreases the quiescent state of hematopoietic stem/progenitor cells and alters the pattern of hematopoietic regeneration after myelosuppression. Blood 117(2):429-439.
35. Ding L, Morrison S J (2013) Haematopoietic stem cells and early lymphoid progenitors occupy distinct bone marrow niches. Nature 495(7440):231-235.
36. Greenbaum A, et al. (2013) CXCL12 in early mesenchymal progenitors is required for haematopoietic stem-cell maintenance. Nature 495(7440):227-30.
37. Cho T-J, Gerstenfeld L C, Einhorn T A (2002) Differential Temporal Expression of Members of the Transforming Growth Factor β Superfamily During Murine Fracture Healing. J Bone Miner Res 17(3):513-520.
38. Maesner C C, Almada A E, Wagers A J (2016) Established cell surface markers efficiently isolate highly overlapping populations of skeletal muscle satellite cells by fluorescence-activated cell sorting. Skelet Muscle 6(1):35.
39. Baldridge M T, King K Y, Boles N C, Weksberg D C, Goodell M A (2010) Quiescent haematopoietic stem cells are activated by IFN-γ in response to chronic infection. Nature 465(7299):793-797.
40. Wilson A, et al. (2008) Hematopoietic Stem Cells Reversibly Switch from Dormancy to Self-Renewal during Homeostasis and Repair. Cell 135(6):1118-1129.
41. Rodgers J T, Schroeder M D, Ma C, Rando T A (2017) HGFA Is an Injury-Regulated Systemic Factor that Induces the Transition of Stem Cells into G Alert. Cell Rep 19(3):479-486.
42. Bodey G P, Buckley M, Sathe Y S, Freireich E J (1966) Quantitative relationships between circulating leukocytes and infection in patients with acute leukemia. Ann Intern Med 64(2):328-40.
43. Lyman G H, et al. (2010) Risk of mortality in patients with cancer who experience febrile neutropenia. Cancer 116(23):5555-5563.
44. Kang R, et al. (2014) HMGB1 in health and disease. Mol Aspects Med 40:1-116.
45. Lee P, Regenbogen S, Gawande A A (2008) How many surgical procedures will Americans experience in an average lifetime?: Evidence from three states. How Many Surgical Procedures Will Americans Experience in an Average Lifetime?: Evidence from Three States (55th Annual Meeting of the Massachusetts Chapter of the American College of Surgeons, Boston, M.A.). Available at: http://www.mcacs.org/abstracts/2008/p15.cgi [Accessed Jun. 6, 2017].
46. Liu L, Cheung T H, Charville G W, Rando T A (2015) Isolation of skeletal muscle stem cells by fluorescence-activated cell sorting. Nat Protoc 10(10):1612-1624.
47. Gregor T, et al. (2012) Correlating Micro-C T Imaging with Quantitative Histology. Injury and Skeletal Biomechanics (InTech). doi:10.5772/48680.
48. Particelli F, et al. (2012) A comparison between micro-C T and histology for the evaluation of cortical bone: effect of polymethylmethacrylate embedding on structural parameters. J Microsc 245(3):302-310.
49. O'Neill K R, et al. (2012) Micro-computed tomography assessment of the progression of fracture healing in mice. Bone 50(6):1357-67.
50. Steiner M, et al. (2015) Comparison between different methods for biomechanical assessment of ex vivo fracture callus stiffness in small animal bone healing studies. PLoS One 10(3):e0119603.
51. Peytour Y, et al. (2010) Obtaining of CD34+ cells from healthy blood donors: development of a rapid and efficient procedure using leukoreduction filters. Transfusion 50(10):2152-2157.
52. Schindelin J, et al. (2012) Fiji: an open-source platform for biological-image analysis. Nat Methods 9(7):676-682.
53. Ilic D and Polak J. Stem cell based therapy—where are we going?Lancet 2012. 379: 877-878.
54. Smith A M et al. “The protein tyrosine phosphatase 1B inhibitor MSI-1436 stimulates regeneration of heart and multiple other tissues.” NPJ Regenerative Medicine 2017. Mar. 3; 2:4. doi: 10.1038/s41536-017-0008-1. eCollection 2017

1. A method of preventing or treating a condition associated with a defect in or damage to an organ or a tissue of such organ in a subject with, or at risk for, such defect or damage to such organ or tissue which comprises administering to the subject a prophylactically or therapeutically effective amount of a fully reduced (all thiol) form of HMGB1 or of a biologically active truncated form of HMGB1, so as to prevent or treat such condition, wherein the subject is, or is anticipated to be, in need of prevention or treatment of the condition in the future, and wherein the organ or tissue relies on repair by stem or parenchymal cells that express the cell surface receptor CXCR4.

2-6. (canceled)

7. The method of claim 1, wherein the organ is the brain, the spinal cord and/or associated nerves, peripheral nerves, blood vessels, an eye, the pancreas, the liver, a lung, the gut, a kidney, the spleen, the bladder, an ureters, or a male or female reproductive organ.

8. The method of claim 7, wherein the organ is the islets of Langerhans region of the pancreas.

9. The method of claim 7, wherein the organ is the small intestine or the large intestine.

10. A method of improving regeneration of blood in a subject comprising administering a therapeutically effective amount of the fully reduced (all thiol) form of HMGB1 or of a biologically active truncated form of HMGB1 so as to improve regeneration of blood, wherein the subject is in need of improved regeneration of blood, or is anticipated to be in need of improved regeneration of blood in the future.

11-13. (canceled)

14. The method of claim 1, wherein the condition is Alzheimer's Disease, Amyotrophic Lateral Sclerosis (Motor Neuron Disease) or Parkinson's Disease.

15. The method of claim 1, wherein the subject is affected by, or at risk for, a stroke.

16. The method of claim 1, wherein the administration is systemic administration.

17. The method of claim 1, wherein the administration is local administration.

18. The method of claim 1, wherein the administration is directly into the subject's coronary artery(s) at the time of insertion of a stent following an acute ischemic episode or myocardical infarction.

19. The method of claim 1, wherein the administration is into the subject's cerebrospinal fluid (intrathecal).

20. (canceled)

21. The method of claim 1, wherein the fully reduced form of HMGB1 is administered to the subject.

22. The method of claim 1, wherein the fully reduced, biologically active truncated form of HMGB1 is administered to the subject.

23. The method of claim 1, wherein the fully reduced form of HMGB1 or of the biologically active truncated form of HMGB1 is
(a) a fully reduced (FR) all-thiol form of HMGB1 (FR-HMGB1) or a biologically active truncated form thereof;
(b) a recombinant non-oxidable one-serine form (1S) of HMGB1 (1S-HMGB1) or a biologically active truncated form thereof, in either case in which a cysteine corresponding to one of C23, C45, or C106 of HMGB1 is replaced by a serine, preferably wherein each of two such cysteines is replaced by a serine;
(c) a recombinant non-oxidable two-serine form (2S) of HMGB1 (2S-HMGB1) or a biologically active truncated form thereof, in either case in which two cysteines corresponding to both C23 and C45 or to both C45 and C106 of HMGB1 are replaced by a serine; or
(d) a recombinant non-oxidable all-serine form (3S) of HMGB1 (3S-HMGB1) or a biologically active truncated form thereof, in either case in which the three cysteines corresponding to each of C23, C45, and C106 of HMGB1 are replaced by a serine.

24-26. (canceled)

27. The method of claim 1, wherein the subject is anticipated to be in need of prevention or treatment at a point in time in the future and the administration of the fully reduced form of HMGB1 or of the biologically active truncated form of HMGB1 is one day to one month prior to said point in time in the future.

28. The method of claim 10, wherein the fully reduced form of HMGB1 is administered to the subject.

29. The method of claim 10, wherein the fully reduced, biologically active truncated form of HMGB1 is administered to the subject.

30. The method of claim 10, wherein the fully reduced form of HMGB1 or of the biologically active truncated form of HMGB1 is
(a) a fully reduced (FR) all-thiol form of HMGB1 (FR-HMGB1) or a biologically active truncated form thereof;
(b) a recombinant non-oxidable one-serine form (1S) of HMGB1 (1S-HMGB1) or a biologically active truncated form thereof, in either case in which a cysteine corresponding to one of C23, C45, or C106 of HMGB1 is replaced by a serine, preferably wherein each of two such cysteines is replaced by a serine;
(c) a recombinant non-oxidable two-serine form (2S) of HMGB1 (2S-HMGB1) or a biologically active truncated form thereof, in either case in which two cysteines corresponding to both C23 and C45 or to both C45 and C106 of HMGB1 are replaced by a serine; or
(d) a recombinant non-oxidable all-serine form (3S) of HMGB1 (3S-HMGB1) or a biologically active truncated form thereof, in either case in which the three cysteines corresponding to each of C23, C45, and C106 of HMGB1 are replaced by a serine.

31. The method of claim 10, wherein the subject is anticipated to be in need of prevention or treatment at a point in time in the future and the administration of the fully reduced form of HMGB1 or of the biologically active truncated form of HMGB1 is one day to one month prior to said point in time in the future.

32. A method of claim 10, wherein the administration is systemic administration or local administration.
